(19) United States
(12) Patent Application Publication
(10) Pub. No.: US 2022/0227789 A1
(43) Pub. Date: Jul. 21, 2022

(54) NOVEL INDOLIZINE-2-CARBOXAMIDES ACTIVE AGAINST THE HEPATITIS B VIRUS (HBV)

(71) Applicant: AiCuris GmbH & Co. KG, Wuppertal (DE)

(72) Inventors: Susanne BONSMANN, Köln (DE); Alastair DONALD, Wuppertal (DE); Andreas URBAN, Sprockhövel (DE); Jasper SPRINGER, Diepenveen (NL); Elena DETTA, Wuppertal (DE)

(73) Assignee: AiCuris GmbH & Co. KG, Wuppertal (DE)

(21) Appl. No.: 17/607,428

(22) PCT Filed: Apr. 29, 2020
(86) PCT No.: PCT/EP2020/061946

§ 371 Date: Oct. 29, 2021

(30) Foreign Application Priority Data
Apr. 30, 2019 (EP) 19172002.8
May 2, 2019 (EP) 19172396.4

(51) Int. Cl. C07D 519/00; C07D 498/04; A61P 31/20

(52) U.S. Cl. CPC C07D 519/00; A61P 31/20; C07D 498/04

(57) ABSTRACT

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes and intermediates for making the compounds.

### TECHNICAL FIELD

The present invention relates generally to novel antiviral agents. Specifically, the present invention relates to compounds which can inhibit the protein(s) encoded by hepatitis B virus (HBV) or interfere with the function of the HBV replication cycle, compositions comprising such compounds, methods for inhibiting HBV viral replication, methods for treating or preventing HBV infection, and processes for making the compounds.

### BACKGROUND OF THE INVENTION

Chronic HBV infection is a significant global health problem, affecting over 5% of the world population (over 350 million people worldwide and 1.25 million individuals in the US). Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact.

The low cure rates of HBV are attributed at least in part to the fact that complete suppression of virus production is difficult to achieve with a single antiviral agent, and to the presence and persistence of covalently closed circular DNA (cccDNA) in the nucleus of infected hepatocytes. However, persistent suppression of HBV DNA slows liver disease progression and helps to prevent hepatocellular carcinoma (HCC).

Current therapy goals for HBV-infected patients are directed to reducing serum HBV DNA to low or undetectable levels, and to ultimately reducing or preventing the development of cirrhosis and HCC.

The HBV is an enveloped, partially double-stranded DNA (dsDNA) virus of the hepadnavirus family (Hepadnaviridae). HBV capsid protein (HBV-CP) plays essential roles in HBV replication. The predominant biological function of HBV-CP is to act as a structural protein to encapsidate pre-genomic RNA and form immature capsid particles, which spontaneously self-assemble from many copies of capsid protein dimers in the cytoplasm.

HBV-CP also regulates viral DNA synthesis through differential phosphorylation states of its C-terminal phosphorylation sites. Also, HBV-CP might facilitate the nuclear translocation of viral relaxed circular genome by means of the nuclear localization signals located in the arginine-rich domain of the C-terminal region of HBV-CP.

In the nucleus, as a component of the viral cccDNA mini-chromosome, HBV-CP could play a structural and regulatory role in the functionality of cccDNA mini-chromosomes. HBV-CP also interacts with viral large envelope protein in the endoplasmic reticulum (ER), and triggers the release of intact viral particles from hepatocytes.

HBV-CP related anti-HBV compounds have been reported. For example, phenylpropenamide derivatives, including compounds named AT-61 and AT-130 (Feld J. et al. Antiviral Res. 2007, 76, 168), and a class of thiazolidin-4-ones from Valeant (WO2006/033995), have been shown to inhibit pre-genomic RNA (pgRNA) packaging.

F. Hoffmann-La Roche AG have disclosed a series of 3-substituted tetrahydro-pyrazolo[1,5-a]pyrazines for the therapy of HBV (WO2016/113273, WO2017/198744, WO2018/011162, WO2018/011160, WO2018/011163).

Shanghai Hengrui Pharma have disclosed a series of heteroaryl piperazines for HBV therapy (WO2019/020070). Shanghai Longwood Biopharmaceuticals have disclosed a series of bicyclic heterocycles active against HBV (WO2018/202155).

Zhimeng Biopharma have disclosed pyrazole-oxazolidinone compounds as being active against HBV (WO2017/173999).

Heteroaryldihydropyrimidines (HAPs) were discovered in a tissue culture-based screening (Weber et al., Antiviral Res. 2002, 54, 69). These HAP analogs act as synthetic allosteric activators and are able to induce aberrant capsid formation that leads to degradation of HBV-CP (WO 99/54326, WO 00/58302, WO 01/45712, WO 01/6840). Further HAP analogs have also been described (J. Med. Chem. 2016, 59 (16), 7651-7666).

A subclass of HAPs from F. Hoffman-La Roche also shows activity against HBV (WO2014/184328, WO2015/132276, and WO2016/146598). A similar subclass from Sunshine Lake Pharma also shows activity against HBV (WO2015/144093). Further HAPs have also been shown to possess activity against HBV (WO2013/102655, Bioorg. Med. Chem. 2017, 25(3) pp. 1042-1056, and a similar subclass from Enanta Therapeutics shows similar activity (WO2017/011552). A further subclass from Medshine Discovery shows similar activity (WO2017/076286). A further subclass (Janssen Pharma) shows similar activity (WO2013/102655).

A subclass of pyridazones and triazinones (F. Hoffman-La Roche) also show activity against HBV (WO2016/023877), as do a subclass of tetrahydropyridopyridines (WO2016/177655). A subclass of tricyclic 4-pyridone-3-carboxylic acid derivatives from Roche also show similar anti-HBV activity (WO2017/013046).

A subclass of sulfamoyl-arylamides from Novira Therapeutics (now part of Johnson & Johnson Inc.) also shows activity against HBV (WO2013/006394, WO2013/096744, WO2014/165128, WO2014/184365, WO2015/109130, WO2016/089990, WO2016/109663, WO2016/109684, WO2016/109689, WO2017/059059). A similar subclass of thioether-arylamides (also from Novira Therapeutics) shows activity against HBV (WO2016/089990). Additionally, a subclass of aryl-azepanes (also from Novira Therapeutics) shows activity against HBV (WO2015/073774). A similar subclass of arylamides from Enanta Therapeutics show activity against HBV (WO2017/015451).

Sulfamoyl derivatives from Janssen Pharma have also been shown to possess activity against HBV (WO2014/033167, WO2014/033170, WO2017/001655, J. Med. Chem, 2018, 61(14) 6247-6260).

A subclass of glyoxamide substituted pyrrolamide derivatives also from Janssen Pharma have also been shown to possess activity against HBV (WO2015/011281). A similar class of glyoxamide substituted pyrrolamides (Gilead Sciences) has also been described (WO2018/039531).

A subclass of sulfamoyl- and oxalyl-heterobiaryls from Enanta Therapeutics also show activity against HBV (WO2016/161268, WO2016/183266, WO2017/015451, WO2017/136403 & US20170253609).

A subclass of aniline-pyrimidines from Assembly Biosciences also show activity against HBV (WO2015/057945, WO2015/172128). A subclass of fused tri-cycles from Assembly Biosciences (dibenzo-thiazepinones, dibenzo-diazepinones, dibenzo-oxazepinones) show activity against HBV (WO2015/138895, WO2017/048950). A further series from Assembly Biosciences (WO2016/168619) also show anti-HBV activity.

A series of cyclic sulfamides has been described as modulators of HBV-CP function by Assembly Biosciences (WO2018/160878).

Arbutus Biopharma have disclosed a series of benzamides for the therapy of HBV (WO2018/052967, WO2018/172852). Also disclosed are compositions and uses of similar compounds in combination with a CYP3A inhibitor (WO2019/046287).

It was also shown that the small molecule bis-ANS acts as a molecular ‘wedge’ and interferes with normal capsid-protein geometry and capsid formation (Zlotnick A et al. J. Virol. 2002, 4848).

Problems that HBV direct acting antivirals may encounter are toxicity, mutagenicity, lack of selectivity, poor efficacy, poor bioavailability, low solubility and difficulty of synthesis. There is a thus a need for additional inhibitors for the treatment, amelioration or prevention of HBV that may overcome at least one of these disadvantages or that have additional advantages such as increased potency or an increased safety window.

Administration of such therapeutic agents to an HBV infected patient, either as monotherapy or in combination with other HBV treatments or ancillary treatments, will lead to significantly reduced virus burden, improved prognosis, diminished progression of the disease and/or enhanced seroconversion rates.

### SUMMARY OF THE INVENTION

Provided herein are compounds useful for the treatment or prevention of HBV infection in a subject in need thereof, and intermediates useful in their preparation. The subject matter of the invention is a compound of Formula I

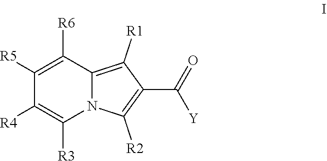

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

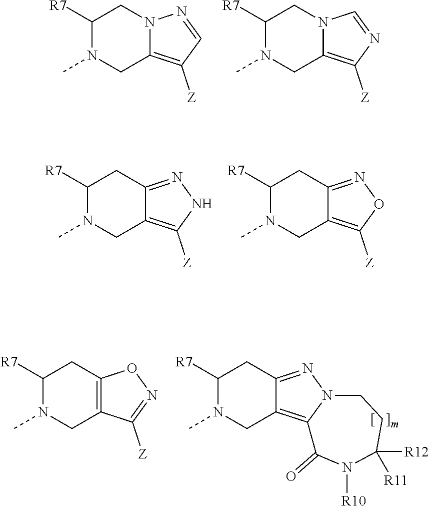

R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

In one embodiment of the invention subject matter of the invention is a compound of Formula I in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

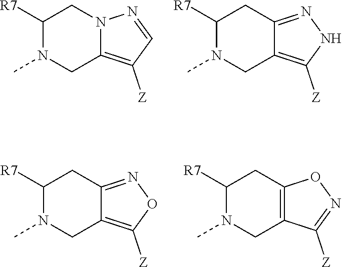

R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

In one embodiment of the invention subject matter of the invention is a compound of Formula I in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

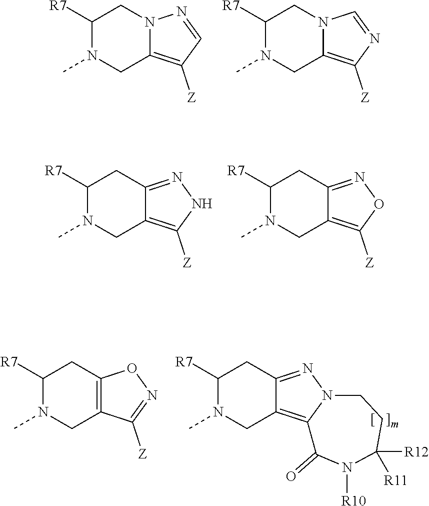

R7 is selected from the group comprising H, D, and C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

In one embodiment of the invention subject matter of the invention is a compound of Formula I in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

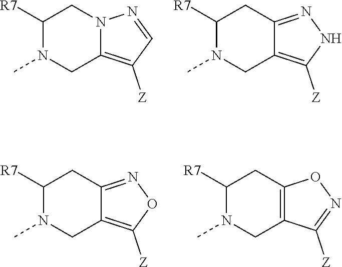

R7 is selected from the group comprising H, D, and C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

In one embodiment of the invention subject matter of the invention is a compound of Formula I in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

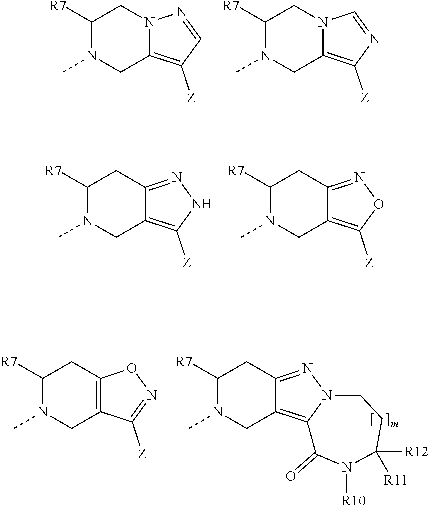

R7 is selected from the group comprising H, D, and C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

In one embodiment of the invention subject matter of the invention are stereoisomers of a compound of Formula I in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising

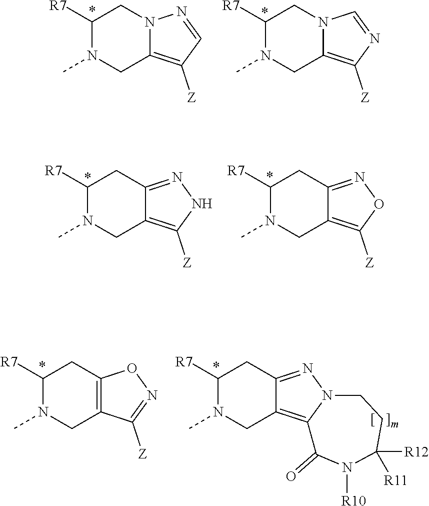

R7 is C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S.

One embodiment of the invention is a compound of Formula I or a pharmaceutically acceptable salt thereof according to the invention, for use in the prevention or treatment of an HBV infection in subject.

One embodiment of the invention is a pharmaceutical composition comprising a compound of Formula I or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula I or a pharmaceutically acceptable salt thereof according to the present invention.

A further embodiment of the invention is a compound of Formula I or a pharmaceutically acceptable salt thereof according to the invention, for use in the prevention or treatment of an HBV infection in subject in need thereof.

A further embodiment of the invention is a compound of Formula Ha or a pharmaceutically acceptable salt thereof according to the invention, for use in the prevention or treatment of an HBV infection in subject in need thereof.

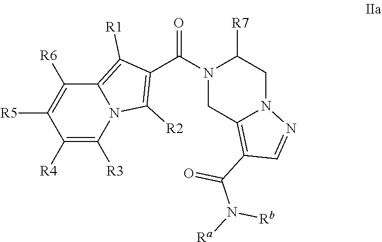

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

In one embodiment of the invention subject matter of the invention is a compound of Formula IIa in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

One embodiment of the invention is a compound of Formula Ha or a pharmaceutically acceptable salt thereof according to the invention, for use in the prevention or treatment of an HBV infection in subject.

One embodiment of the invention is a pharmaceutical composition comprising a compound of Formula Ha or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Ha or a pharmaceutically acceptable salt thereof according to the present invention.

A further embodiment of the invention is a compound of Formula Ha or a pharmaceutically acceptable salt thereof according to the invention, for use in the prevention or treatment of an HBV infection in subject in need thereof.

A further embodiment of the invention is a compound of Formula IIb or a pharmaceutically acceptable salt thereof according to the invention, for use in the prevention or treatment of an HBV infection in subject in need thereof.

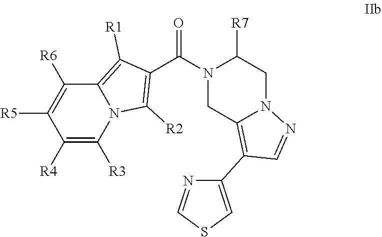

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

In one embodiment of the invention subject matter of the invention is a compound of Formula IIb in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

One embodiment of the invention is a compound of Formula IIb or a pharmaceutically acceptable salt thereof according to the invention, for use in the prevention or treatment of an HBV infection in subject.

One embodiment of the invention is a pharmaceutical composition comprising a compound of Formula IIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIb or a pharmaceutically acceptable salt thereof according to the present invention.

A further embodiment of the invention is a compound of Formula IIb or a pharmaceutically acceptable salt thereof according to the invention, for use in the prevention or treatment of an HBV infection in subject in need thereof.

A further embodiment of the invention is a compound of Formula IIc or a pharmaceutically acceptable salt thereof according to the invention, for use in the prevention or treatment of an HBV infection in subject in need thereof.

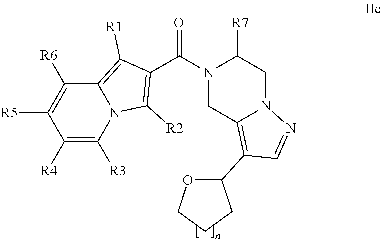

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention is a compound of Formula IIc in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3.

In one embodiment of the invention subject matter of the invention are stereoisomers of a compound of Formula IIc

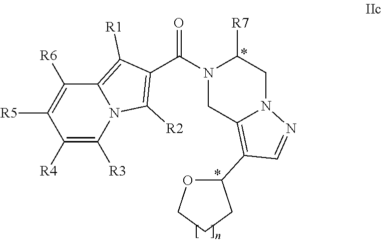

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3.

One embodiment of the invention is a compound of Formula IIc or a pharmaceutically acceptable salt thereof according to the invention, for use in the prevention or treatment of an HBV infection in subject.

One embodiment of the invention is a pharmaceutical composition comprising a compound of Formula IIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIc or a pharmaceutically acceptable salt thereof according to the present invention.

A further embodiment of the invention is a compound of Formula IIc or a pharmaceutically acceptable salt thereof according to the invention, for use in the prevention or treatment of an HBV infection in subject in need thereof.

A further embodiment of the invention is a compound of Formula Ma or a pharmaceutically acceptable salt thereof according to the invention, for use in the prevention or treatment of an HBV infection in subject in need thereof.

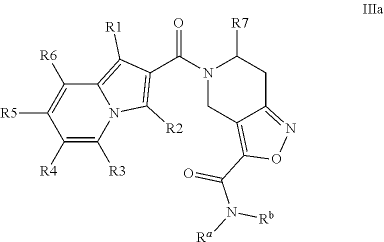

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

In one embodiment of the invention subject matter of the invention is a compound of Formula IIIa in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

One embodiment of the invention is a compound of Formula Ma or a pharmaceutically acceptable salt thereof according to the invention, for use in the prevention or treatment of an HBV infection in subject.

One embodiment of the invention is a pharmaceutical composition comprising a compound of Formula Ina or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Ma or a pharmaceutically acceptable salt thereof according to the present invention.

A further embodiment of the invention is a compound of Formula Ma or a pharmaceutically acceptable salt thereof according to the invention, for use in the prevention or treatment of an HBV infection in subject in need thereof.

A further embodiment of the invention is a compound of Formula IIIb or a pharmaceutically acceptable salt thereof according to the invention, for use in the prevention or treatment of an HBV infection in subject in need thereof.

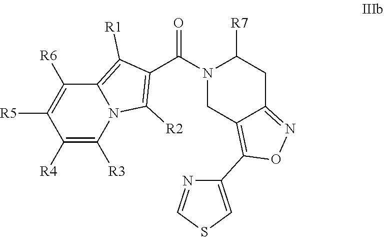

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

In one embodiment of the invention subject matter of the invention is a compound of Formula IIIb in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

One embodiment of the invention is a compound of Formula IIIb or a pharmaceutically acceptable salt thereof according to the invention, for use in the prevention or treatment of an HBV infection in subject.

One embodiment of the invention is a pharmaceutical composition comprising a compound of Formula IIIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IIIb or a pharmaceutically acceptable salt thereof according to the present invention.

A further embodiment of the invention is a compound of Formula IIIb or a pharmaceutically acceptable salt thereof according to the invention, for use in the prevention or treatment of an HBV infection in subject in need thereof.

A further embodiment of the invention is a compound of Formula Mc or a pharmaceutically acceptable salt thereof according to the invention, for use in the prevention or treatment of an HBV infection in subject in need thereof.

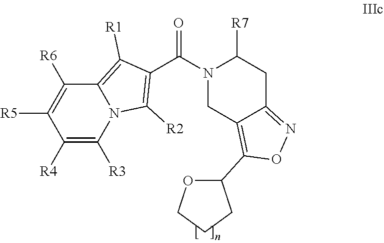

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention is a compound of Formula IIIc in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention are stereoisomers of a compound of Formula IIIc

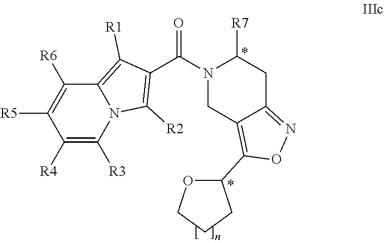

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3.

One embodiment of the invention is a compound of Formula Mc or a pharmaceutically acceptable salt thereof according to the invention, for use in the prevention or treatment of an HBV infection in subject.

One embodiment of the invention is a pharmaceutical composition comprising a compound of Formula IIIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Mc or a pharmaceutically acceptable salt thereof according to the present invention.

A further embodiment of the invention is a compound of Formula Mc or a pharmaceutically acceptable salt thereof according to the invention, for use in the prevention or treatment of an HBV infection in subject in need thereof.

A further embodiment of the invention is a compound of Formula IVa or a pharmaceutically acceptable salt thereof according to the invention, for use in the prevention or treatment of an HBV infection in subject in need thereof.

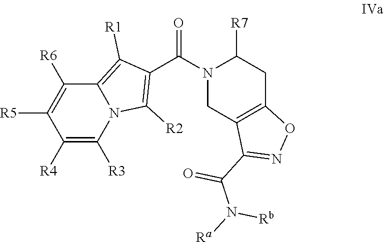

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

In one embodiment of the invention subject matter of the invention is a compound of Formula IVa in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

One embodiment of the invention is a compound of Formula IVa or a pharmaceutically acceptable salt thereof according to the invention, for use in the prevention or treatment of an HBV infection in subject.

One embodiment of the invention is a pharmaceutical composition comprising a compound of Formula IVa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVa or a pharmaceutically acceptable salt thereof according to the present invention.

A further embodiment of the invention is a compound of Formula IVa or a pharmaceutically acceptable salt thereof according to the invention, for use in the prevention or treatment of an HBV infection in subject in need thereof.

A further embodiment of the invention is a compound of Formula IVb or a pharmaceutically acceptable salt thereof according to the invention, for use in the prevention or treatment of an HBV infection in subject in need thereof.

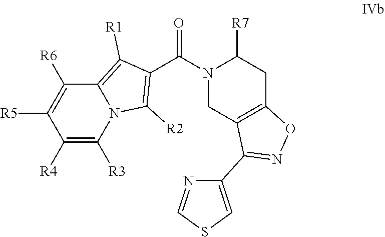

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

In one embodiment of the invention subject matter of the invention is a compound of Formula IVb in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

One embodiment of the invention is a compound of Formula IVb or a pharmaceutically acceptable salt thereof according to the invention, for use in the prevention or treatment of an HBV infection in subject.

One embodiment of the invention is a pharmaceutical composition comprising a compound of Formula IVb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVb or a pharmaceutically acceptable salt thereof according to the present invention.

A further embodiment of the invention is a compound of Formula IVb or a pharmaceutically acceptable salt thereof according to the invention, for use in the prevention or treatment of an HBV infection in subject in need thereof.

A further embodiment of the invention is a compound of Formula IVc or a pharmaceutically acceptable salt thereof according to the invention, for use in the prevention or treatment of an HBV infection in subject in need thereof.

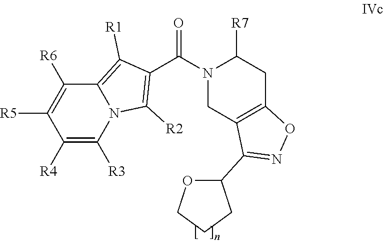

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention is a compound of Formula IVc in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention are stereoisomers of a compound of Formula IVc

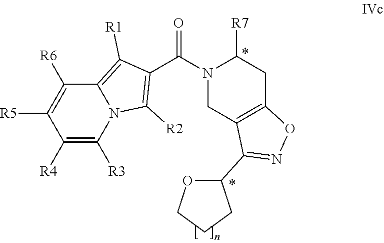

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

One embodiment of the invention is a compound of Formula IVc or a pharmaceutically acceptable salt thereof according to the invention, for use in the prevention or treatment of an HBV infection in subject.

One embodiment of the invention is a pharmaceutical composition comprising a compound of Formula IVc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula IVc or a pharmaceutically acceptable salt thereof according to the present invention.

A further embodiment of the invention is a compound of Formula IVc or a pharmaceutically acceptable salt thereof according to the invention, for use in the prevention or treatment of an HBV infection in subject in need thereof.

A further embodiment of the invention is a compound of Formula Va or a pharmaceutically acceptable salt thereof according to the invention, for use in the prevention or treatment of an HBV infection in subject in need thereof.

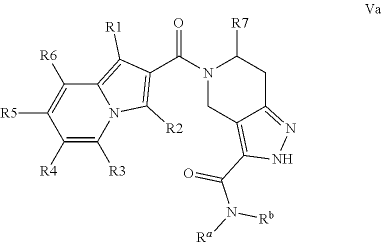

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

In one embodiment of the invention subject matter of the invention is a compound of Formula Va in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

One embodiment of the invention is a compound of Formula Va or a pharmaceutically acceptable salt thereof according to the invention, for use in the prevention or treatment of an HBV infection in subject.

One embodiment of the invention is a pharmaceutical composition comprising a compound of Formula Va or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Va or a pharmaceutically acceptable salt thereof according to the present invention.

A further embodiment of the invention is a compound of Formula Va or a pharmaceutically acceptable salt thereof according to the invention, for use in the prevention or treatment of an HBV infection in subject in need thereof.

A further embodiment of the invention is a compound of Formula Vb or a pharmaceutically acceptable salt thereof according to the invention, for use in the prevention or treatment of an HBV infection in subject in need thereof.

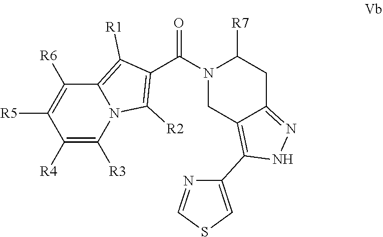

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

In one embodiment of the invention subject matter of the invention is a compound of Formula Vb in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

One embodiment of the invention is a compound of Formula Vb or a pharmaceutically acceptable salt thereof according to the invention, for use in the prevention or treatment of an HBV infection in subject.

One embodiment of the invention is a pharmaceutical composition comprising a compound of Formula Vb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Vb or a pharmaceutically acceptable salt thereof according to the present invention.

A further embodiment of the invention is a compound of Formula Vb or a pharmaceutically acceptable salt thereof according to the invention, for use in the prevention or treatment of an HBV infection in subject in need thereof.

A further embodiment of the invention is a compound of Formula Vc or a pharmaceutically acceptable salt thereof according to the invention, for use in the prevention or treatment of an HBV infection in subject in need thereof.

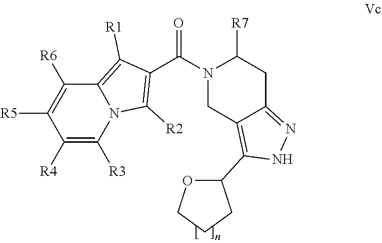

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention is a compound of Formula Vc in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention are stereoisomers of a compound of Formula Vc

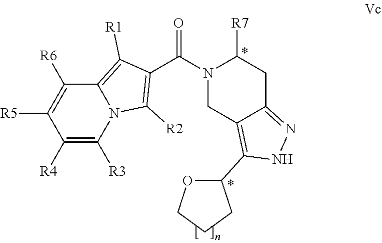

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

One embodiment of the invention is a compound of Formula Vc or a pharmaceutically acceptable salt thereof according to the invention, for use in the prevention or treatment of an HBV infection in subject.

One embodiment of the invention is a pharmaceutical composition comprising a compound of Formula Vc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula Vc or a pharmaceutically acceptable salt thereof according to the present invention.

A further embodiment of the invention is a compound of Formula Vc or a pharmaceutically acceptable salt thereof according to the invention, for use in the prevention or treatment of an HBV infection in subject in need thereof.

A further embodiment of the invention is a compound of Formula VIa or a pharmaceutically acceptable salt thereof according to the invention, for use in the prevention or treatment of an HBV infection in subject in need thereof.

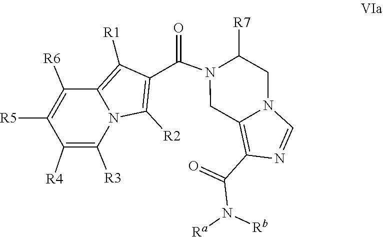

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

In one embodiment of the invention subject matter of the invention is a compound of Formula VIa in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano.

One embodiment of the invention is a compound of Formula VIa or a pharmaceutically acceptable salt thereof according to the invention, for use in the prevention or treatment of an HBV infection in subject.

One embodiment of the invention is a pharmaceutical composition comprising a compound of Formula VIa or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIa or a pharmaceutically acceptable salt thereof according to the present invention.

A further embodiment of the invention is a compound of Formula VIa or a pharmaceutically acceptable salt thereof according to the invention, for use in the prevention or treatment of an HBV infection in subject in need thereof.

A further embodiment of the invention is a compound of Formula VIb or a pharmaceutically acceptable salt thereof according to the invention, for use in the prevention or treatment of an HBV infection in subject in need thereof.

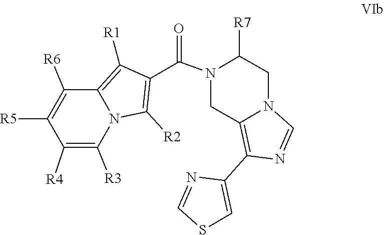

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

In one embodiment of the invention subject matter of the invention is a compound of Formula VIb in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl

One embodiment of the invention is a compound of Formula VIb or a pharmaceutically acceptable salt thereof according to the invention, for use in the prevention or treatment of an HBV infection in subject.

One embodiment of the invention is a pharmaceutical composition comprising a compound of Formula VIb or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIb or a pharmaceutically acceptable salt thereof according to the present invention.

A further embodiment of the invention is a compound of Formula VIb or a pharmaceutically acceptable salt thereof according to the invention, for use in the prevention or treatment of an HBV infection in subject in need thereof.

A further embodiment of the invention is a compound of Formula VIc or a pharmaceutically acceptable salt thereof according to the invention, for use in the prevention or treatment of an HBV infection in subject in need thereof.

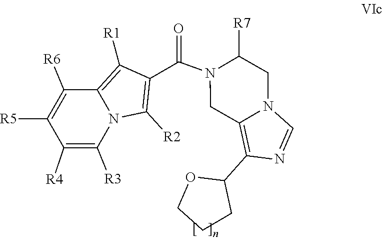

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention is a compound of Formula VIc in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

In one embodiment of the invention subject matter of the invention are stereoisomers of a compound of Formula VIc

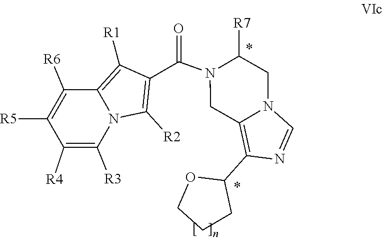

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3

One embodiment of the invention is a compound of Formula VIc or a pharmaceutically acceptable salt thereof according to the invention, for use in the prevention or treatment of an HBV infection in subject.

One embodiment of the invention is a pharmaceutical composition comprising a compound of Formula VIc or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VIc or a pharmaceutically acceptable salt thereof according to the present invention.

A further embodiment of the invention is a compound of Formula VIc or a pharmaceutically acceptable salt thereof according to the invention, for use in the prevention or treatment of an HBV infection in subject in need thereof.

A further embodiment of the invention is a compound of Formula VII or a pharmaceutically acceptable salt thereof according to the invention, for use in the prevention or treatment of an HBV infection in subject in need thereof.

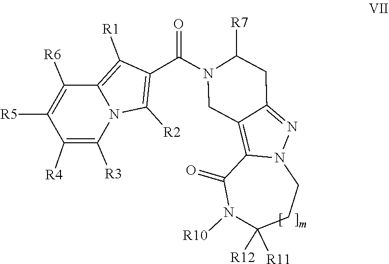

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3

In one embodiment of the invention subject matter of the invention is a compound of Formula VII in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3

In one embodiment of the invention subject matter of the invention are stereoisomers of a compound of Formula VII

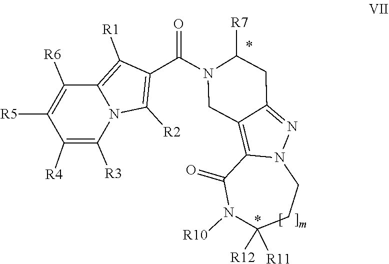

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3

One embodiment of the invention is a compound of Formula VII or a pharmaceutically acceptable salt thereof according to the invention, for use in the prevention or treatment of an HBV infection in subject.

One embodiment of the invention is a pharmaceutical composition comprising a compound of Formula VII or a pharmaceutically acceptable salt thereof according to the present invention, together with a pharmaceutically acceptable carrier.

One embodiment of the invention is a method of treating an HBV infection in an individual in need thereof, comprising administering to the individual a therapeutically effective amount of a compound of Formula VII or a pharmaceutically acceptable salt thereof according to the present invention.

A further embodiment of the invention is a compound of Formula VII or a pharmaceutically acceptable salt thereof according to the invention, for use in the prevention or treatment of an HBV infection in subject in need thereof.

In some embodiments, the dose of a compound of the invention is from about 1 mg to about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., another drug for HBV treatment) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof. All before mentioned doses refer to daily doses per patient.

In general it is contemplated that an antiviral effective daily amount would be from about 0.01 to about 50 mg/kg, or about 0.01 to about 30 mg/kg body weight. It may be appropriate to administer the required dose as two, three, four or more sub-doses at appropriate intervals throughout the day. Said sub-doses may be formulated as unit dosage forms, for example containing about 1 to about 500 mg, or about 1 to about 300 mg or about 1 to about 100 mg, or about 2 to about 50 mg of active ingredient per unit dosage form.

The compounds of the invention may, depending on their structure, exist as salts, solvates or hydrates. The invention therefore also encompasses the salts, solvates or hydrates and respective mixtures thereof.

The compounds of the invention may, depending on their structure, exist in tautomeric or stereoisomeric forms (enantiomers, diastereomers). The invention therefore also encompasses the tautomers, enantiomers or diastereomers and respective mixtures thereof. The stereoisomerically uniform constituents can be isolated in a known manner from such mixtures of enantiomers and/or diastereomers.

Subject-matter of the present invention is a compound of Formula I, IIa, IIb, IIc, IIIa, IIIb, IIIc, IVa, IVb, IVc, Va, Vb, Vc, VIa, VIb, VIc, VII or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug.

Subject-matter of the present invention is a compound of Formula I, IIa, IIb, IIc, IIIa, IIIb, IVa, IVb, IVc, Va, Vb, Vc, VIa, VIb, VIc, VII or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug for use in the prevention or treatment of an HBV infection in subject.

Subject-matter of the present invention is also a pharmaceutical composition comprising a compound of Formula I, IIa, IIb, IIc, IIIa, IIIb, IIIc, IVa, IVb, IVc, Va, Vb, Vc, VIa, VIb, VIc, VII or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug, together with a pharmaceutically acceptable carrier.

Subject-matter of the present invention is also a method of treating an HBV infection in an individual in need thereof, comprising administering to the individual a therapeutically effective amount of a compound of Formula I, IIa, IIb, IIc, IIIa, IIIb, IIIc, IVa, IVb, IVc, Va, Vb, Vc, VIa, VIb, VIc, VII or a pharmaceutically acceptable salt thereof or a solvate or a hydrate of said compound or a pharmaceutically acceptable salt of said solvate or hydrate or a prodrug of said compound or a pharmaceutically acceptable salt of said prodrug or a solvate or a hydrate of said prodrug or a pharmaceutically acceptable salt of said solvate or a hydrate of said prodrug.

Subject matter of the present invention is also a method of preparing the compounds of the present invention. Subject matter of the invention is, thus, a method for the preparation of a compound of Formula I according to the present invention by reacting a compound of Formula VIII

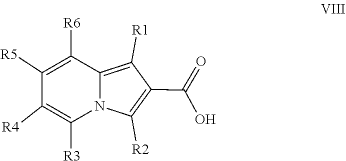

in which R1, R2, R3, R4, R5 and R6 are as above-defined, with a compound selected from

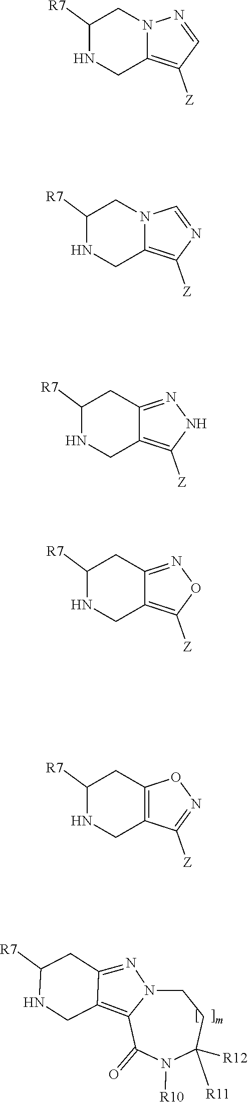

in which R7, R10, R11, R12, m and Z are as above defined.

In one embodiment, a method for the preparation of a compound of Formula I according to the present invention by reacting a compound of Formula VIII

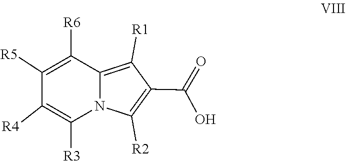

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro

with a compound selected from

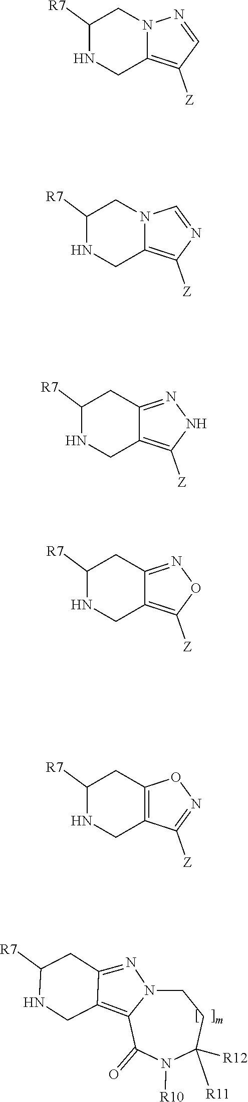

in which
R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3.

In one embodiment, a method for the preparation of a compound of Formula I according to the present invention by reacting a compound of Formula VIII

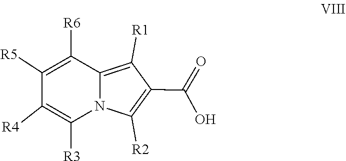

in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro

with a compound selected from

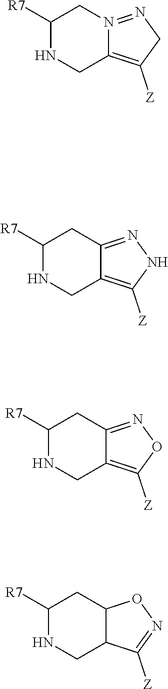

in which
R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3.

### Definitions

Listed below are definitions of various terms used to describe this invention. These definitions apply to the terms as they are used throughout this specification and claims unless otherwise limited in specific instances either individually or as part of a larger group.

Unless defined otherwise all technical and scientific terms used herein generally have the same meaning as commonly understood by one of ordinary skill in the art to which this invention belongs. Generally the nomenclature used herein and the laboratory procedures in cell culture, molecular genetics, organic chemistry and peptide chemistry are those well known and commonly employed in the art.

As used herein the articles “a” and “an” refer to one or to more than one (i.e. to at least one) of the grammatical object of the article. By way of example, “an element” means one element or more than one element. Furthermore, use of the term “including” as well as other forms such as “include”, “includes” and “included”, is not limiting.

As used herein the term “capsid assembly modulator” refers to a compound that disrupts or accelerates or inhibits or hinders or delays or reduces or modifies normal capsid assembly (e.g. during maturation) or normal capsid disassembly (e.g. during infectivity) or perturbs capsid stability, thereby inducing aberrant capsid morphology or aberrant capsid function. In one embodiment, a capsid assembly modulator accelerates capsid assembly or disassembly thereby inducing aberrant capsid morphology. In another embodiment a capsid assembly modulator interacts (e.g. binds at an active site, binds at an allosteric site or modifies and/or hinders folding and the like), with the major capsid assembly protein (HBV-CP), thereby disrupting capsid assembly or disassembly. In yet another embodiment a capsid assembly modulator causes a perturbation in the structure or function of HBV-CP (e.g. the ability of HBV-CP to assemble, disassemble, bind to a substrate, fold into a suitable conformation or the like which attenuates viral infectivity and/or is lethal to the virus).

As used herein the term “treatment” or “treating” is defined as the application or administration of a therapeutic agent i.e., a compound of the invention (alone or in combination with another pharmaceutical agent) to a patient, or application or administration of a therapeutic agent to an isolated tissue or cell line from a patient (e.g. for diagnosis or ex vivo applications) who has an HBV infection, a symptom of HBV infection, or the potential to develop an HBV infection with the purpose to cure, heal, alleviate, relieve, alter, remedy, ameliorate, improve or affect the HBV infection, the symptoms of HBV infection or the potential to develop an HBV infection. Such treatments may be specifically tailored or modified based on knowledge obtained from the field of pharmacogenomics.

As used herein the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein the term “patient”, “individual” or “subject” refers to a human or a non-human mammal. Non-human mammals include for example livestock and pets such as ovine, bovine, porcine, feline, and murine mammals. Preferably the patient, subject, or individual is human.

As used herein the terms “effective amount”, “pharmaceutically effective amount”, and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein the term “pharmaceutically acceptable” refers to a material such as a carrier or diluent which does not abrogate the biological activity or properties of the compound and is relatively non-toxic i.e. the material may be administered to an individual without causing undesirable biological effects or interacting in a deleterious manner with any of the components of the composition in which it is contained.

As used herein the term “pharmaceutically acceptable salt” refers to derivatives of the disclosed compounds wherein the parent compound is modified by converting an existing acid or base moiety to its salt form. Examples of pharmaceutically acceptable salts include but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent or in a mixture of the two; generally nonaqueous media like ether, ethyl acetate, ethanol, isopropanol, or acetonitrile are preferred. Lists of suitable salts are found in Remington's Pharmaceutical Sciences 17th ed. Mack Publishing Company, Easton, Pa., 1985 p. 1418 and Journal of Pharmaceutical Science, 66, 2 (1977), each of which is incorporated herein by reference in its entirety. Pharmaceutically acceptable salts of the compounds according to the invention include acid addition salts, for example, but not limited to, salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid. Pharmaceutically acceptable salts of the compounds according to the invention also include salts of customary bases, for example, but not limited to, alkali metal salts (for example sodium and potassium salts), alkaline earth metal salts (for example calcium and magnesium salts) and ammonium salts derived from ammonia or organic amines having 1 to 16 carbon atoms, such as, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

As used herein, the term “solvate” refers to the compounds which form a complex in the solid or liquid state by coordination with solvent molecules. Suitable solvents include, but are not limited to, methanol, ethanol, acetic acid and water. Hydrates are a special form of solvates in which the coordination takes place with water.

As used herein the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including but not limited to intravenous, oral, aerosol, rectal, parenteral, ophthalmic, pulmonary and topical administration.

As used herein the term “pharmaceutically acceptable carrier” means a pharmaceutically acceptable material, composition or carrier such as a liquid or solid filler, stabilizer, dispersing agent, suspending agent, diluent, excipient, thickening agent, solvent or encapsulating material involved in carrying or transporting a compound useful within the invention within or to the patient such that it may perform its intended function. Typically such constructs are carried or transported from one organ, or portion of the body, to another organ or portion of the body. Each carrier must be “acceptable” in the sense of being compatible with the other ingredients of the formulation including the compound use within the invention and not injurious to the patient. Some examples of materials that may serve as pharmaceutically acceptable carriers include: sugars, such as lactose, glucose and sucrose; starches such as corn starch and potato starch; cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols such as propylene glycol; polyols such as glycerin, sorbitol, mannitol and polyethylene glycol; esters such as ethyl oleate and ethyl laurate; agar; buffering agents, such as magnesium hydroxide and aluminium hydroxide; surface active agents; alginic acid; pyrogen-free water; isotonic saline; Ringer's solution; ethyl alcohol; phosphate buffer solutions and other non-toxic compatible substances employed in pharmaceutical formulations. As used herein “pharmaceutically acceptable carrier” also includes any and all coatings, antibacterial and antifungal agents and absorption delaying agents and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described for example in Remington's Pharmaceutical Sciences (Genaro, Ed., Mack Publishing Company, Easton, Pa., 1985) which is incorporated herein by reference.

As used herein, the term “substituted” means that an atom or group of atoms has replaced hydrogen as the substituent attached to another group.

As used herein, the term “comprising” also encompasses the option “consisting of”.

As used herein, the term “alkyl” by itself or as part of another substituent means, unless otherwise stated, a straight or branched chain hydrocarbon having the number of carbon atoms designated (i.e. C1-C6-alkyl means one to six carbon atoms) and includes straight and branched chains. Examples include methyl, ethyl, propyl, isopropyl, butyl, isobutyl, tert-butyl, pentyl, neopentyl, and hexyl. In addition, the term “alkyl” by itself or as part of another substituent can also mean a C1-C3 straight chain hydrocarbon substituted with a C3-C5-carbocylic ring. Examples include (cyclopropyl)methyl, (cyclobutyl)methyl and (cyclopentyl)methyl. For the avoidance of doubt, where two alkyl moieties are present in a group, the alkyl moieties may be the same or different.

As used herein the term “alkenyl” denotes a monovalent group derived from a hydrocarbon moiety containing at least two carbon atoms and at least one carbon-carbon double bond of either E or Z stereochemistry. The double bond may or may not be the point of attachment to another group. Alkenyl groups (e.g. C2-C8-alkenyl) include, but are not limited to for example ethenyl, propenyl, prop-1-en-2-yl, butenyl, methyl-2-buten-1-yl, heptenyl and octenyl. For the avoidance of doubt, where two alkenyl moieties are present in a group, the alkyl moieties may be the same or different.

As used herein, a C2-C6-alkynyl group or moiety is a linear or branched alkynyl group or moiety containing from 2 to 6 carbon atoms, for example a C2-C4 alkynyl group or moiety containing from 2 to 4 carbon atoms. Exemplary alkynyl groups include —C≡CH or —CH₂—C≡C, as well as 1- and 2-butynyl, 2-pentynyl, 3-pentynyl, 4-pentynyl, 2-hexynyl, 3-hexynyl, 4-hexynyl and 5-hexynyl. For the avoidance of doubt, where two alkynyl moieties are present in a group, they may be the same or different.

As used herein, the term “halo” or “halogen” alone or as part of another substituent means unless otherwise stated a fluorine, chlorine, bromine, or iodine atom, preferably fluorine, chlorine, or bromine, more preferably fluorine or chlorine. For the avoidance of doubt, where two halo moieties are present in a group, they may be the same or different.

As used herein, a C1-C6-alkoxy group or C2-C6-alkenyloxy group is typically a said C1-C6-alkyl (e.g. a C1-C4 alkyl) group or a said C2-C6-alkenyl (e.g. a C2-C4 alkenyl) group respectively which is attached to an oxygen atom.

As used herein the term “aryl” employed alone or in combination with other terms, means unless otherwise stated a carbocyclic aromatic system containing one or more rings (typically one, two or three rings) wherein such rings may be attached together in a pendant manner such as a biphenyl, or may be fused, such as naphthalene. Examples of aryl groups include phenyl, anthracyl, and naphthyl. Preferred examples are phenyl (e.g. C6-aryl) and biphenyl (e.g. C12-aryl). In some embodiments aryl groups have from six to sixteen carbon atoms. In some embodiments aryl groups have from six to twelve carbon atoms (e.g. C6-C12-aryl). In some embodiments, aryl groups have six carbon atoms (e.g. C6-aryl).

As used herein the terms “heteroaryl” and “heteroaromatic” refer to a heterocycle having aromatic character containing one or more rings (typically one, two or three rings). Heteroaryl substituents may be defined by the number of carbon atoms e.g. C1-C9-heteroaryl indicates the number of carbon atoms contained in the heteroaryl group without including the number of heteroatoms. For example a C1-C9-heteroaryl will include an additional one to four heteroatoms. A polycyclic heteroaryl may include one or more rings that are partially saturated. Non-limiting examples of heteroaryls include:

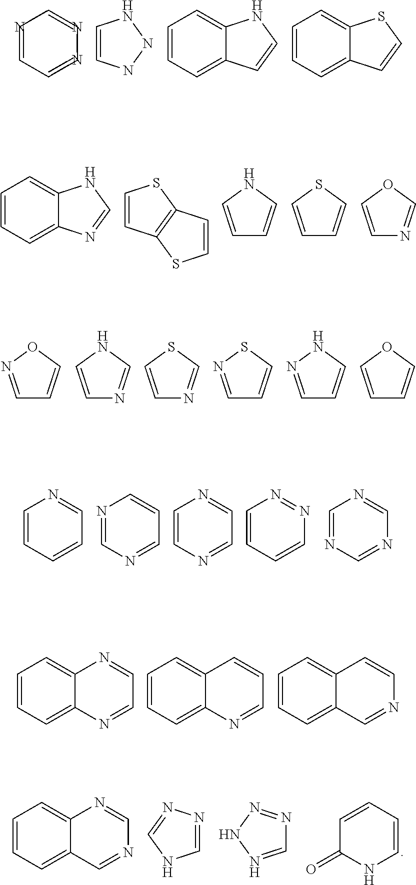

Additional non-limiting examples of heteroaryl groups include pyridyl, pyrazinyl, pyrimidinyl (including e.g. 2- and 4-pyrimidinyl), pyridazinyl, thienyl, furyl, pyrrolyl (including e.g., 2-pyrrolyl), imidazolyl, thiazolyl, oxazolyl, pyrazolyl (including e.g. 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl. Non-limiting examples of polycyclic heterocycles and heteroaryls include indolyl (including 3-, 4-, 5-, 6- and 7-indolyl), indolinyl, quinolyl, tetrahydroquinolyl, isoquinolyl (including, e.g. 1- and 5-isoquinolyl), 1,2,3,4-tetrahydroisoquinolyl, cinnolinyl, quinoxalinyl (including, e.g 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (including, e.g. 3-, 4-, 5-, 6-, and 7-benzofuryl), 2,3-dihydrobenzofuryl, 1,2-benzisoxazolyl, benzothienyl (including e.g. 3-, 4-, 5-, 6-, and 7-benzothienyl), benzoxazolyl, benzothiazolyl (including e.g. 2-benzothiazolyl and 5-benzothiazolyl), purinyl, benzimidazolyl (including e.g., 2-benzimidazolyl), benzotriazolyl, thioxanthinyl, carbazolyl, carbolinyl, acridinyl, pyrrolizidinyl and quinolizidinyl.

As used herein, the terms “pyridyl”, “pyrimidinyl”, “pyrazinyl”, “pyridazinyl”, “triazinyl”, “oxazolyl”, “isoxazolyl”, “imidazolyl”, and “pyrazolyl” when employed alone or in combination with one or more other terms encompasses, unless otherwise stated, positional isomers thereof.

As used herein an unsubstituted said pyridyl includes 2-pyridyl, 3-pyridyl and 4-pyridyl. Examples of substituted pyridyl includes said 2-pyridyl, wherein further substitutions can be at the 3-, 4-, 5- or 6-positions. Further examples of substituted pyridyl also includes said 3-pyridyl, wherein further substitutions can be at the 2-, 4-, 5- or 6-positions, and said 4-pyridyl, wherein further substitutions can be at the 2-, 3-, 5- or 6-positions.

As used herein an unsubstituted said pyrimidinyl includes 2-pyrimidinyl, 4-pyrimidinyl and 5-pyrimidinyl. Examples of substituted pyrimidinyl includes said 2-pyrimidinyl, wherein further substitutions are on the 4-, 5- or 6-positions. Examples of substituted pyrimidinyl also includes said 4-pyrimidinyl, wherein further substitutions are on the 2-, 5- or 6-positions. Examples of substituted pyrimidinyl also includes said 5-pyrimidinyl, wherein further substitutions are on the 2-, 4- or 6-positions.

As used herein an unsubstituted said pyrazinyl is 2-pyrazinyl. Examples of substituted pyrazinyl include said 2-pyrimidinyl, wherein further substitutions are on the 3-, 5- or 6-positions.

As used herein an unsubstituted said pyridazinyl is 3-pyridazinyl. Examples of substituted pyrazinyl include said 3-pyrimidinyl, wherein further substitutions are on the 4-, 5- or 6-positions.

As used herein an unsubstituted said triazinyl is 2-triazinyl. A substituted triazinyl is a said 2-triazinyl with further substitutions on the 4- or 6-positions.

As used herein an unsubstituted said oxazolyl includes 2-oxazolyl and 4-oxazolyl. A substituted oxazolyl is either a said 2-oxazolyl with further substitutions on the 4- or 5-positions, or a said 4-oxazolyl with further substitutions on the 2-, or 5-positions.

As used herein an unsubstituted said isoxazolyl includes 3-isoxazolyl and 4-isoxazolyl. A substituted isoxazolyl is either a said 3-oxazolyl with further substitutions on the 4- or 5-positions, or a said 4-oxazolyl with further substitutions on the 3-, or 5-positions.

As used herein an unsubstituted said triazolyl includes 1,2,3-triazolyl, 1,2,4-triazolyl and 1,3,4-triazolyl.

As used herein an unsubstituted said imidazolyl includes 2-imidazolyl and 4-imidazolyl. A substituted imidazolyl is either a said 2-imidazolyl with further substitutions on the N1-, N3-, 4- or 5-positions with the proviso that only one of N1- and N3-may be substituted, or a said 4-imidazolyl with further substitutions on the N1-, 2-, N3- or 5-positions, with the proviso that only one of N1- and N3-may be substituted.

As used herein an unsubstituted said pyrazolyl includes 3-pyrazolyl and 4-pyrazolyl. A substituted pyrazolyl is either a said 3-pyrazolyl with further substitutions on the N1-, N2-, 4- or 5-positions with the proviso that only one of N1- and N2-may be substituted, or a said 4-pyrazolyl with further substitutions on the N1-, N2-, 3- or 5-positions with the proviso that only one of N1- and N2-may be substituted.

As used herein the term “haloalkyl” is typically a said alkyl, alkenyl, alkoxy or alkenoxy group respectively wherein any one or more of the carbon atoms is substituted with one or more said halo atoms as defined above. Haloalkyl embraces monohaloalkyl, dihaloalkyl, and polyhaloalkyl radicals. The term “haloalkyl” includes but is not limited to fluoromethyl, 1-fluoroethyl, difluoromethyl, 2,2-difluoroethyl, 2,2-difluoropropyl, 2,2,2-trifluoroethyl, trifluoromethyl, chloromethyl, dichloromethyl, trichloromethyl, pentafluoroethyl, difluoromethoxy, and trifluoromethoxy.

As used herein, a C1-C6-hydroxyalkyl group is a said C1-C6 alkyl group substituted by one or more hydroxy groups. Typically, it is substituted by one, two or three hydroxyl groups. Preferably, it is substituted by a single hydroxy group.

As used herein, a C1-C6-aminoalkyl group is a said C1-C6 alkyl group substituted by one or more amino groups. Typically, it is substituted by one, two or three amino groups. Preferably, it is substituted by a single amino group.

As used herein, the term “carboxy” and by itself or as part of another substituent means, unless otherwise stated, a group of formula C(═O)OH.

As used herein, the term “carboxyl ester” by itself or as part of another substituent means, unless otherwise stated, a group of formula C(═O)OX, wherein X is selected from the group consisting of C1-C6-alkyl, C3-C7-cycloalkyl, and aryl.

As used herein, a C1-C4-carboxyalkyl group is a said C1-C4 alkyl group substituted by said carboxy group.

As used herein, a C1-C4-carboxamidoalkyl group is a said C1-C4 alkyl group substituted by a substituted or unsubstituted carboxamide group.

As used herein, a C1-C4-acylsulfonamido-alkyl group is a said C1-C4 alkyl group substituted by an acylsulfonamide group of general formula C(═O)NHSO₂CH₃ or C(═O)NHSO₂-c-Pr.

As used herein the term “cycloalkyl” refers to a monocyclic or polycyclic nonaromatic group wherein each of the atoms forming the ring (i.e. skeletal atoms) is a carbon atom. In one embodiment, the cycloalkyl group is saturated or partially unsaturated. In another embodiment, the cycloalkyl group is fused with an aromatic ring. Cycloalkyl groups include groups having 3 to 10 ring atoms (C3-C10-cycloalkyl), groups having 3 to 8 ring atoms (C3-C8-cycloalkyl), groups having 3 to 7 ring atoms (C3-C7-cycloalkyl) and groups having 3 to 6 ring atoms (C3-C6-cycloalkyl). Illustrative examples of cycloalkyl groups include, but are not limited to the following moieties:

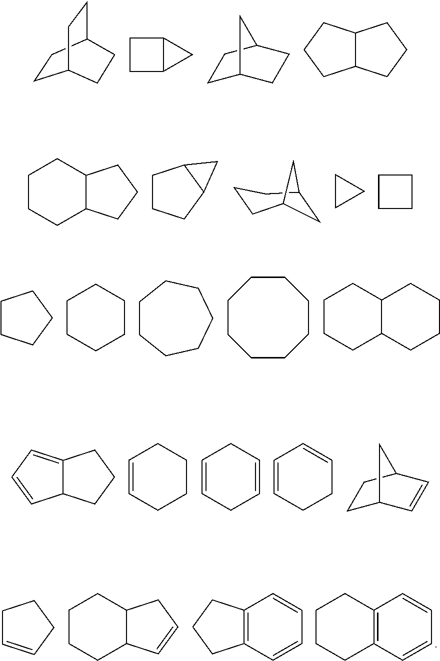

Monocyclic cycloalkyls include but are not limited to cyclopropyl, cyclobutyl, cyclopentyl, cyclohexyl, cycloheptyl, and cyclooctyl. Dicyclic cycloalkyls include but are not limited to tetrahydronaphthyl, indanyl, and tetrahydropentalene. Polycyclic cycloalkyls include adamantine and norbornane. The term cycloalkyl includes “unsaturated nonaromatic carbocyclyl” or “nonaromatic unsaturated carbocyclyl” groups both of which refer to a nonaromatic carbocycle as defined herein which contains at least one carbon-carbon double bond or one carbon-carbon triple bond.

As used herein the term “halo-cycloalkyl” is typically a said cycloalkyl wherein any one or more of the carbon atoms is substituted with one or more said halo atoms as defined above. Halo-cycloalkyl embraces monohaloalkyl, dihaloalkyl, and polyhaloalkyl radicals. Halo-cycloalkyl embraces 3,3-difluoro-cyclobutyl, 3-fluorocyclobutyl, 2-fluorocyclobutyl, 2,2-difluorocyclobutyl, and 2,2-difluorocyclopropyl.

As used herein the terms “heterocycloalkyl” and “heterocyclyl” refer to a heteroalicyclic group containing one or more rings (typically one, two or three rings), that contains one to four ring heteroatoms each selected from oxygen, sulfur and nitrogen. In one embodiment each heterocyclyl group has from 3 to 10 atoms in its ring system with the proviso that the ring of said group does not contain two adjacent oxygen or sulfur atoms. In one embodiment each heterocyclyl group has a fused bicyclic ring system with 3 to 10 atoms in the ring system, again with the proviso that the ring of said group does not contain two adjacent oxygen or sulfur atoms. In one embodiment each heterocyclyl group has a bridged bicyclic ring system with 3 to 10 atoms in the ring system, again with the proviso that the ring of said group does not contain two adjacent oxygen or sulfur atoms. In one embodiment each heterocyclyl group has a spiro-bicyclic ring system with 3 to 10 atoms in the ring system, again with the proviso that the ring of said group does not contain two adjacent oxygen or sulfur atoms. Heterocyclyl substituents may be alternatively defined by the number of carbon atoms e.g. C2-C8-heterocyclyl indicates the number of carbon atoms contained in the heterocyclic group without including the number of heteroatoms. For example a C2-C8-heterocyclyl will include an additional one to four heteroatoms. In another embodiment the heterocycloalkyl group is fused with an aromatic ring. In another embodiment the heterocycloalkyl group is fused with a heteroaryl ring. In one embodiment the nitrogen and sulfur heteroatoms may be optionally oxidized and the nitrogen atom may be optionally quaternized. The heterocyclic system may be attached, unless otherwise stated, at any heteroatom or carbon atom that affords a stable structure. An example of a 3-membered heterocyclyl group includes and is not limited to aziridine. Examples of 4-membered heterocycloalkyl groups include, and are not limited to azetidine and a beta-lactam. Examples of 5-membered heterocyclyl groups include, and are not limited to pyrrolidine, oxazolidine and thiazolidinedione. Examples of 6-membered heterocycloalkyl groups include, and are not limited to, piperidine, morpholine, piperazine, N-acetylpiperazine and N-acetylmorpholine. Other non-limiting examples of heterocyclyl groups are

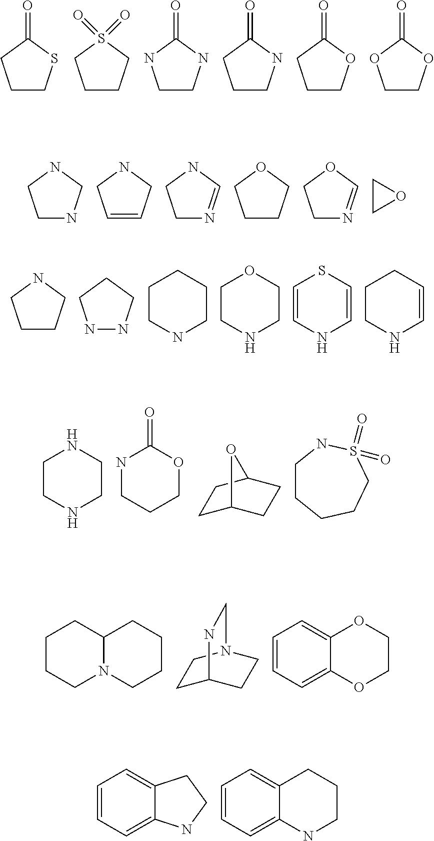

Examples of heterocycles include monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, tetrahydropyrane, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, 1,3-dioxolane, homopiperazine, homopiperidine, 1,3-dioxepane, 47-dihydro-1,3-dioxepin, and hexamethyleneoxide. The terms “C3-C7-heterocycloalkyl” includes but is not limited to tetrahydrofuran-2-yl, tetrahydrofuran-3-yl, 3-oxabicyclo[3.1.0]hexan-6-yl, 3-azabicyclo[3.1.0]hexan-6-yl, tetrahydropyran-4-yl, tetrahydropyran-3-yl, tetrahydropyran-2-yl, and azetidin-3-yl.

As used herein, the term “aromatic” refers to a carbocycle or heterocycle with one or more polyunsaturated rings and having aromatic character i.e. having (4n+2) delocalized π(pi) electrons where n is an integer.

As used herein, the term “acyl”, employed alone or in combination with other terms, means, unless otherwise stated, to mean to an alkyl, cycloalkyl, heterocycloalkyl, aryl or heteroaryl group linked via a carbonyl group.

As used herein, the terms “carbamoyl” and “substituted carbamoyl”, employed alone or in combination with other terms, means, unless otherwise stated, to mean a carbonyl group linked to an amino group optionally mono or di-substituted by hydrogen, alkyl, cycloalkyl, heterocycloalkyl, aryl or heteroaryl. In some embodiments, the nitrogen substituents will be connected to form a heterocyclyl ring as defined above.

As used herein the term “cyano”, employed alone or in combination with other terms, means, unless otherwise stated, a nitrogen atom triple bonded to a carbon atom, in which the carbon atom is further attached to another atom (—C≡N).

As used herein the term “nitro”, employed alone or in combination with other terms, means, unless otherwise stated, a nitrogen atom bonded to two oxygen atoms, in which the nitrogen atom is further attached to another atom (—NO₂).

As used herein the term “amino”, employed alone or in combination with other terms, means, unless otherwise stated, to mean a nitrogen atom single bonded to two hydrogen atoms in which the nitrogen atom is further attached to another atom (—NH₂).

The term “prodrug” refers to a precursor of a drug that is a compound which upon administration to a patient, must undergo chemical conversion by metabolic processes before becoming an active pharmacological agent. Illustrative prodrugs of compounds in accordance with Formula I are esters and amides, preferably alkyl esters of fatty acid esters. Prodrug formulations here comprise all substances which are formed by simple transformation including hydrolysis, oxidation or reduction either enzymatically, metabolically or in any other way. A suitable prodrug contains e.g. a substance of general formula I bound via an enzymatically cleavable linker (e.g. carbamate, phosphate, N-glycoside or a disulfide group) to a dissolution-improving substance (e.g. tetraethylene glycol, saccharides, formic acids or glucuronic acid, etc.). Such a prodrug of a compound according to the invention can be applied to a patient, and this prodrug can be transformed into a substance of general formula I so as to obtain the desired pharmacological effect.

### EXAMPLES

The invention is now described with reference to the following Examples. These Examples are provided for the purpose of illustration only, and the invention is not limited to these Examples, but rather encompasses all variations that are evident as a result of the teachings provided herein.

The required substituted indolizine-2-carboxylic acids may be prepared in a number of ways; the main routes employed being outlined in Schemes 1-3. To the chemist skilled in the art it will be apparent that there are other methodologies that will also achieve the preparation of these intermediates.

Substituted indolizine-2-carboxylic acids may be prepared by the Morita-Bayliss-Hilmann reaction on suitably substituted pyridine-2-carbaldehydes, as shown in Scheme 1.

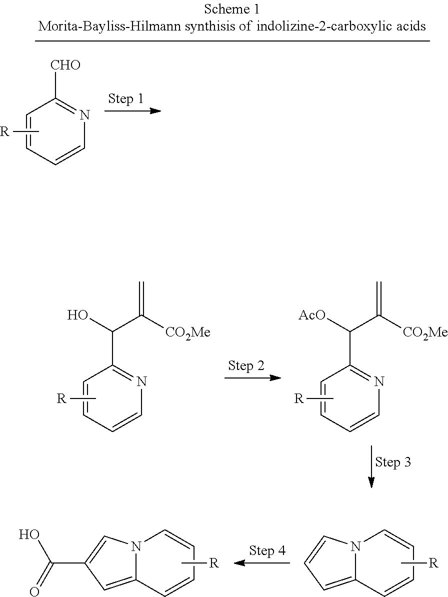

In Step 1 of Scheme 1, a suitably functionalized pyridine-2-carbaldehyde is reacted with methyl prop-2-enoate (methyl acrylate) and a tertiary amine e.g. 1,4-diazabicyclo[2.2.2]octane (DABCO) to give the Morita-Bayliss-Hillman adduct. In Step 2, this adduct is then acylated by, for example, acetic anhydride to give the ester, which is then cyclized under heating in Step 3 to give the indolizine-2-carboxylic acid ester. Hydrolysis of the ester in Step 4 with, for example, aqueous sodium hydroxide gives the desired indolizine-2-carboxylic acid.

Substituted indolizine-2-carboxylic acids may also be prepared by the Chichibabin reaction, using suitably functionalized 2-methyl-pyridines (2-picolines) as shown in Scheme 2.

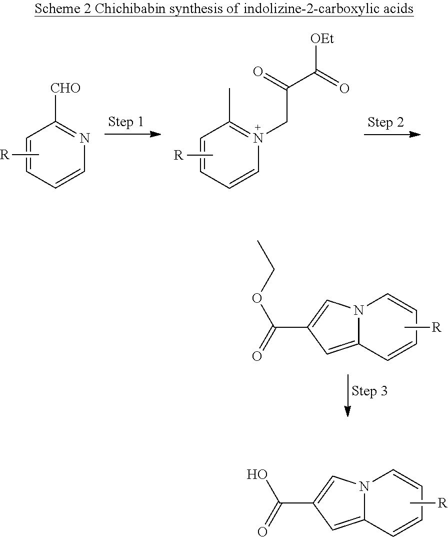

In Step 1 of Scheme 2, a suitably functionalized 2-methyl-pyridine (picoline) is reacted with an ester of bromopyruvic acid, for example ethyl bromopyruvate (as drawn) or tert-butyl 3-bromo-2-oxopropionate, to give the pyridinium salt. This adduct is then cyclized under basic conditions in Step 2 by, for example, caesium carbonate to give the indolizine ester. Hydrolysis of the carboxylic acid ester in Step 3 with, for example, aqueous sodium hydroxide gives the desired indolizine-2-carboxylic acid.

Substituted indolizine-2-carboxylic acids may also be prepared by the functionalization of a suitably substituted indolizine as shown in Scheme 3.

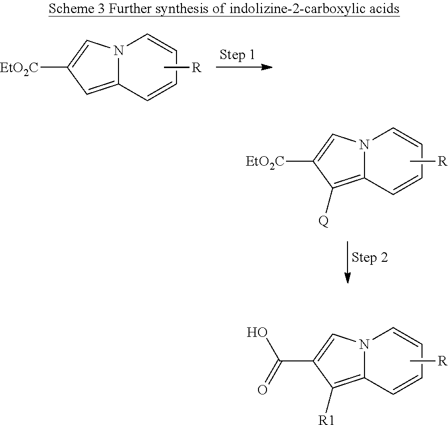

In Step 1 of Scheme 3, a suitably functionalized indolizine is reacted with a formylating or halogenating agent, for example N-bromo-succinimide or bromine, to give a substituted indolizine. This adduct can then be further functionalized by methods well known in the art in Step 2 by, for example, metalation-quenching, palladium catalysed cross-coupling reaction, or Wittig reaction. Hydrolysis of the carboxylic acid ester in Step 3 with, for example, aqueous sodium hydroxide gives an indolizine-2-carboxylic acid.

The HBV core protein modulators can be prepared in a number of ways. Schemes 4-19 illustrate the main routes employed for their preparation for the purpose of this application. To the chemist skilled in the art it will be apparent that there are other methodologies that will also achieve the preparation of these intermediates and Examples.

In a further embodiment, compounds of Formula I can be prepared as shown in Scheme 4 below

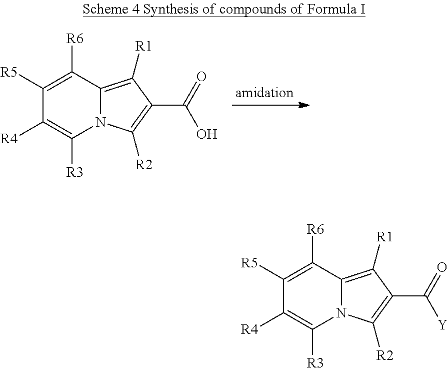

The carboxylic acid described in Scheme 4 is amidated in step 1 with methods known in literature (A. El-Faham, F. Albericio, Chem. Rev. 2011, 111, 6557-6602), e.g. with HATU resulting in compounds of Formula I.

In a further embodiment, compounds of Formula Ha can be prepared as shown in Scheme 5.

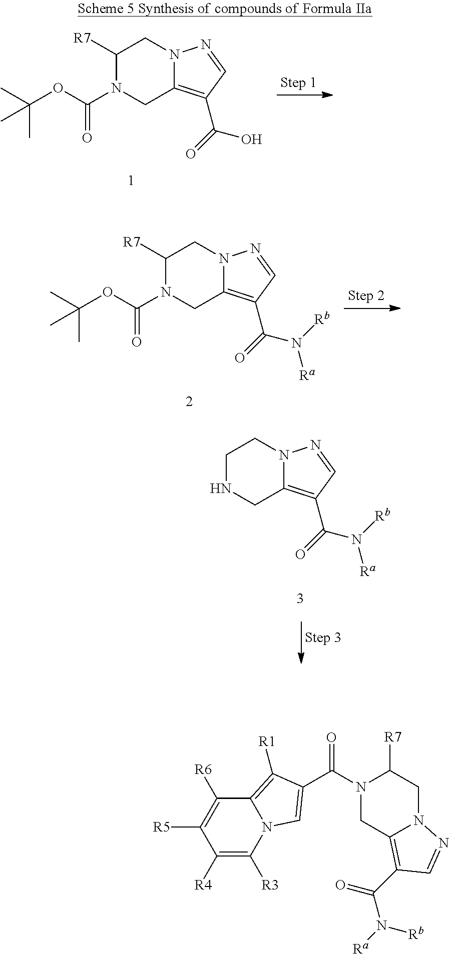

Compound 1 described in Scheme 5 is in step 1 coupled with an amine with methods known in literature (A. El-Faham, F. Albericio, Chem. Rev. 2011, 111, 6557-6602), e.g. with HATU to give a compound with the general structure 2. The nitrogen protective group of compound 2 in Scheme 5 is in step 2 deprotected (WO2018/011162, A. Isidro-Llobet et al., Chem. Rev., 2009, 109, 2455-2504), drawn as but not limited to Boc, e.g. with HCl to give an amine of general structure 3. An amide coupling in step 3 with methods known in literature (A. El-Faham, F. Albericio, Chem. Rev. 2011, 111, 6557-6602), e.g. with HATU results in compounds of Formula IIa.

In a further embodiment, compounds of Formula IIb can be prepared as shown in Scheme 6.

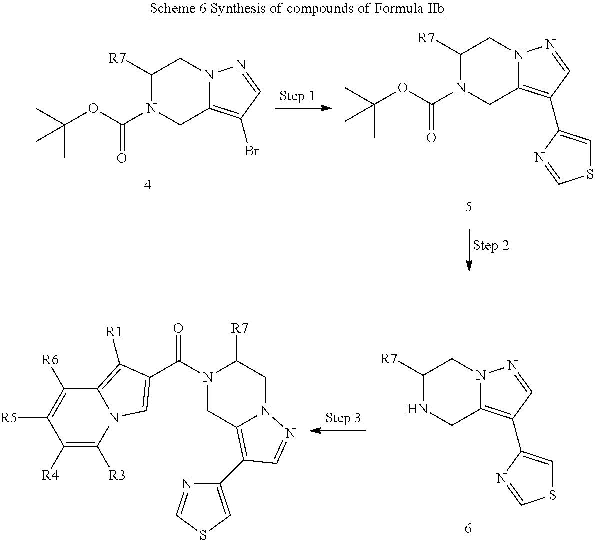

In Step 1 in Scheme 6 a transition metal catalysed cross coupling reaction on compound 4 (drawn as but not limited to a bromo substituted aromatic) with, for example 4-(tributylstannyl)-1,3-thiazole gives a compound of general structure 5 (WO2018/124060). The nitrogen protective group of compound 5 in Scheme 6 is in step 2 deprotected (WO2018/011162, A. Isidro-Llobet et al., Chem. Rev., 2009, 109, 2455-2504), drawn as but not limited to Boc, e.g. with HCl to give an amine of general structure 6. An amide coupling in step 3 with methods known in literature (A. El-Faham, F. Albericio, Chem. Rev. 2011, 111, 6557-6602), e.g. with HATU results in compounds of Formula IIb.

In a further embodiment, compounds of Formula IIc can be prepared as shown in Scheme 7.

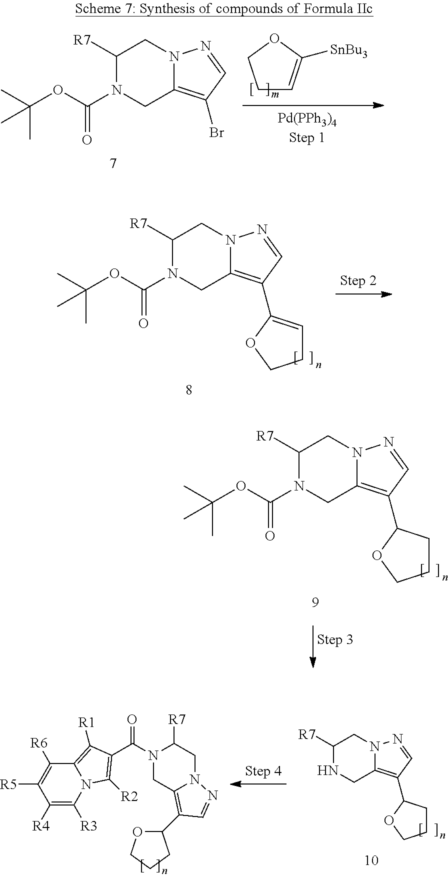

Compound 7 described in Scheme 7 (drawn as but not limited to a bromo substituted aromatic) is in step 1 coupled with a organo-metallate (drawn as, but not limited to a dihydrofuran-2-yl tributyl tin) under palladium catalysis e.g. with Pd(PPh₃)₄ to give compounds of general structure 8. Reduction of the double bond e.g. with H₂ and palladium on carbon gives compounds of general structure 9. The nitrogen protective group of 9 in Scheme 7 is in step 3 deprotected (WO2018/011162, A. Isidro-Llobet et al., Chem. Rev., 2009, 109, 2455-2504), drawn as but not limited to Boc, e.g. with HCl to give an amine of general structure 10. An amide coupling in step 4 with methods known in literature (A. El-Faham, F. Albericio, Chem. Rev. 2011, 111, 6557-6602), e.g. with HATU results in compounds of Formula IIc.

In a further embodiment, compounds of Formula IVa can be prepared as shown in Scheme 8.

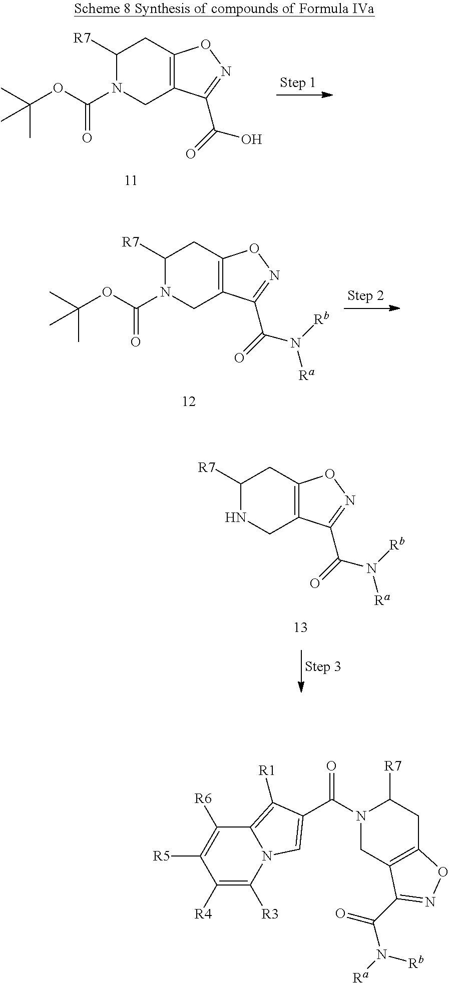

Compound 11 described in Scheme 8 is in step 1 coupled with an amine with methods known in literature (A. El-Faham, F. Albericio, Chem. Rev. 2011, 111, 6557-6602), e.g. with HATU to give a compound with the general structure 12. The nitrogen protective group of compound 12 in Scheme 8 is in step 2 deprotected (WO2018/011162, A. Isidro-Llobet et al., Chem. Rev., 2009, 109, 2455-2504), drawn as but not limited to Boc, e.g. with HCl to give an amine of general structure 13. An amide coupling in step 3 with methods known in literature (A. El-Faham, F. Albericio, Chem. Rev. 2011, 111, 6557-6602), e.g. with HATU results in compounds of Formula IVa.

In a further embodiment, compounds of Formula Va can be prepared as shown in Scheme 9.

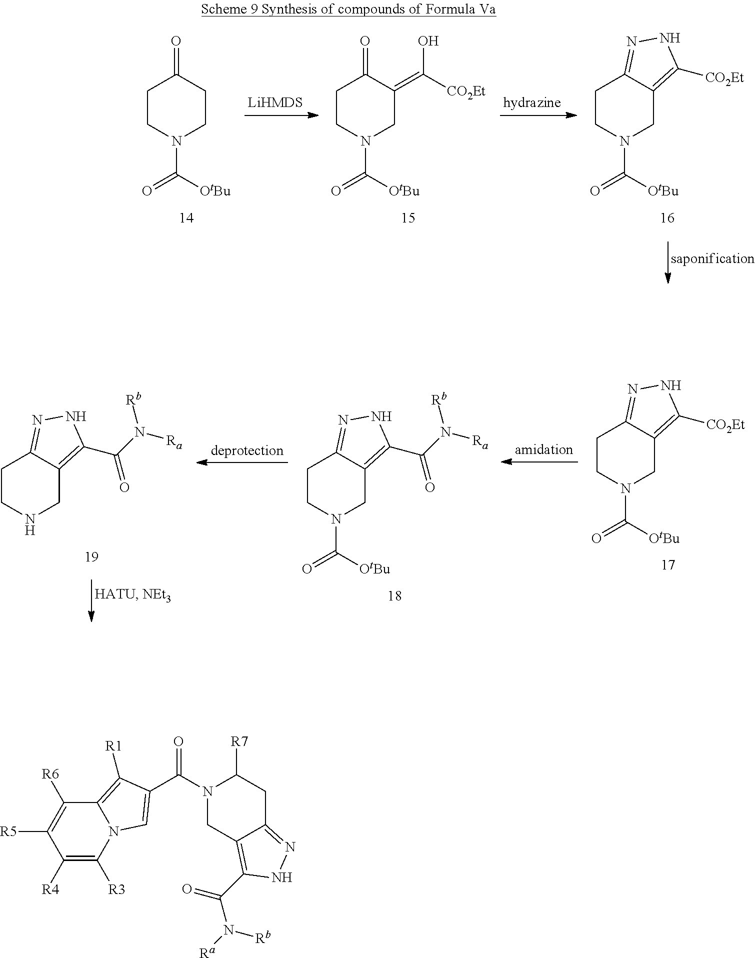

In step 1 ketone 14 in Scheme 9 is converted into compound 15 under basic conditions (WO200722280). Compound 15 is cyclized in step 2 with hydrazine into pyrazole 16 (WO200722280). The ester of compound 16, drawn as but not limited to ethyl, is hydrolysed by methods known from the literature (WO200722280) to give acid 17. By methods known from the literature, acid 17 in step 4 is amidated (A. El-Faham, F. Albericio, Chem. Rev. 2011, 111, 6557-6602), to give compounds with the general structure 18. In step 5 deprotection of the nitrogen protective group (A. Isidro-Llobet et al., Chem. Rev., 2009, 109, 2455-2504), drawn as but not limited to Boc, e.g. with TFA gives amine 19. An amide coupling in step 6 with methods known in literature (A. El-Faham, F. Albericio, Chem. Rev. 2011, 111, 6557-6602), e.g. with HATU results in compounds of Formula Va.

In a further embodiment, compounds of Formula Va can be prepared as shown in Scheme 10.

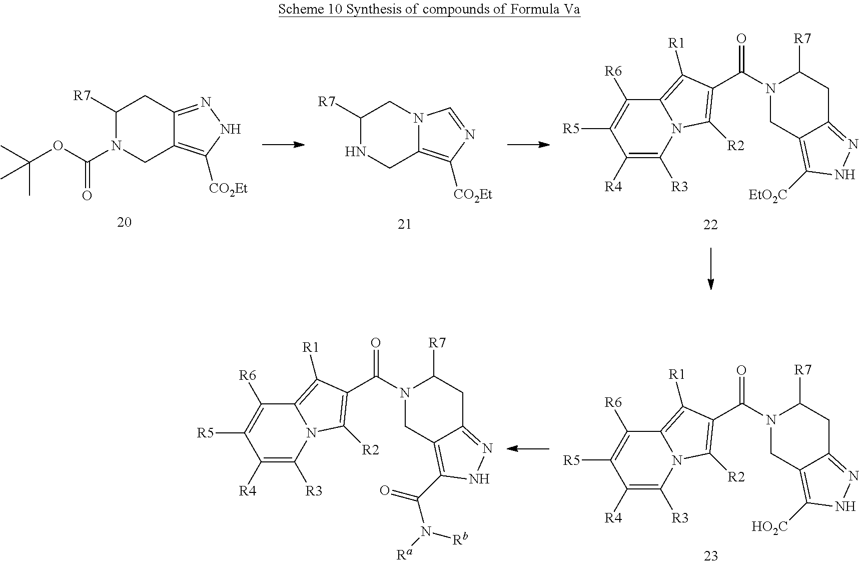

In step 1 deprotection of the nitrogen protective group (A. Isidro-Llobet et al., Chem. Rev., 2009, 109, 2455-2504) from compound 20 described in Scheme 10, drawn as but not limited to Boc, e.g. with HCl gives amine 21. An amide coupling in step 2 with methods known in literature (A. El-Faham, F. Albericio, Chem. Rev. 2011, 111, 6557-6602), e.g. with HATU results in a compound with the general structure 22. In step 3 the ester of compound 22, drawn as but not limited to ethyl, is hydrolysed by methods known from the literature (WO200722280) to give acid 23. By methods known from the literature, acid 23 in step 4 is amidated (A. El-Faham, F. Albericio, Chem. Rev. 2011, 111, 6557-6602) to give compounds of Formula Va.

In a further embodiment, compounds of Formula VIa can be prepared as shown in Scheme 11.

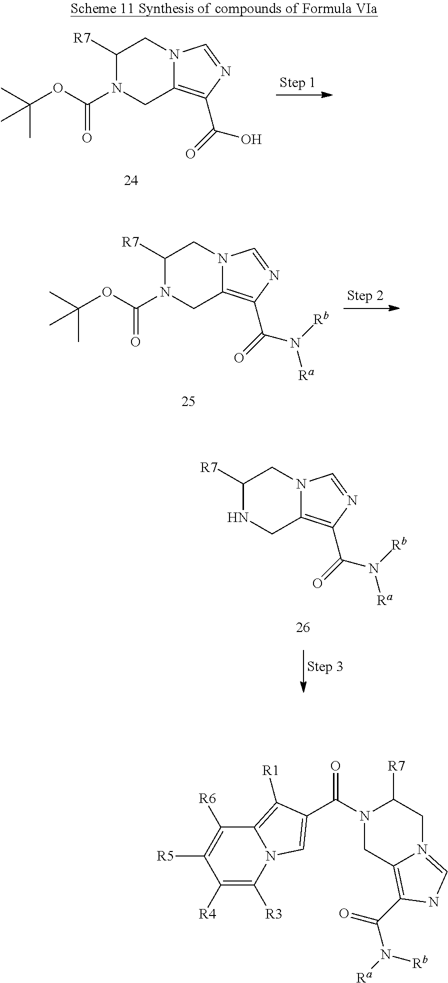

Compound 24 described in Scheme 11 is in step 1 coupled with an amine with methods known in literature (A. El-Faham, F. Albericio, Chem. Rev. 2011, 111, 6557-6602), e.g. with HATU to give a compound with the general structure 25. The nitrogen protective group of compound 25 in Scheme 11 is in step 2 deprotected (WO2018/011162, A. Isidro-Llobet et al., Chem. Rev., 2009, 109, 2455-2504), drawn as but not limited to Boc, e.g. with HCl to give an amine of general structure 26. An amide coupling in step 3 with methods known in literature (A. El-Faham, F. Albericio, Chem. Rev. 2011, 111, 6557-6602), e.g. with HATU results in compounds of Formula VIa.

In a further embodiment, compounds of Formula VII can be prepared as shown in Scheme 12.

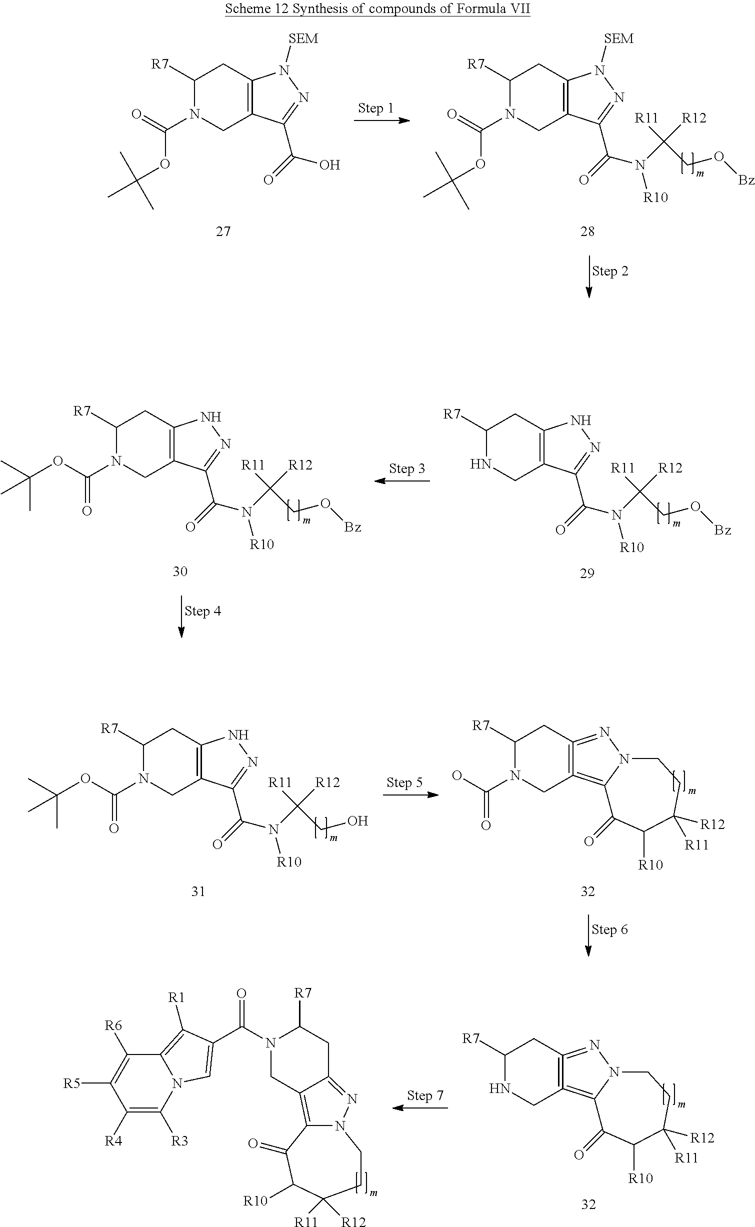

Compound 27 described in Scheme 12 is amidated in step 1 with methods known in literature (A. El-Faham, F. Albericio, Chem. Rev. 2011, 111, 6557-6602), e.g. with HATU resulting in compounds of general structure 28. Two of the three protecting groups (drawn as but not limited to Boc and SEM) are then removed in step 2 with, for example, HCl give a compound of general structure 29. The amine group is then re-protected in step 3 with a protecting group orthogonal to the alcohol protecting group (drawn as but not limited to benzoyl) as for example, a Boc group to give a compound of general structure 30. Removal of the alcohol protecting group, drawn as, but not limited to benzoyl with, for example, aqueous sodium hydroxide gives a compound of general structure 31. In step 5, Mitsunobu reaction of the alcohol with the pyrazole NH (WO2005/120516) gives a compound of general structure 32, which can then be deprotected (drawn as but not limited to Boc), with, for example HCl, to give a compound of general structure 33. The amine group of 33 can then be acylated with methods known in literature (A. El-Faham, F. Albericio, Chem. Rev. 2011, 111, 6557-6602), e.g. with HATU resulting in compounds of Formula VII.

In a further embodiment, compounds of Formula Ma can be prepared as shown in Scheme 13.

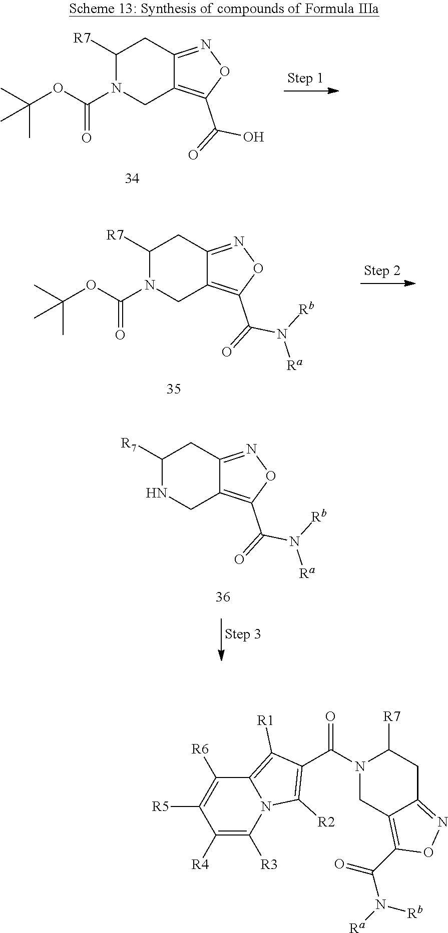

Compound 34 described in Scheme 13 is in step 1 coupled with an amine with methods known in literature (A. El-Faham, F. Albericio, Chem. Rev. 2011, 111, 6557-6602), e.g. with HATU to give a compound with the general structure 35. The nitrogen protective group of compound 35 in Scheme 13 is in step 2 deprotected (WO2018/011162, A. Isidro-Llobet et al., Chem. Rev., 2009, 109, 2455-2504), drawn as but not limited to Boc, e.g. with HCl to give an amine of general structure 36. An amide coupling in step 3 with methods known in literature (A. El-Faham, F. Albericio, Chem. Rev. 2011, 111, 6557-6602), e.g. with HATU results in compounds of Formula IIIa.

In a further embodiment, compounds of Formula IIIb can be prepared as shown in Scheme 14.

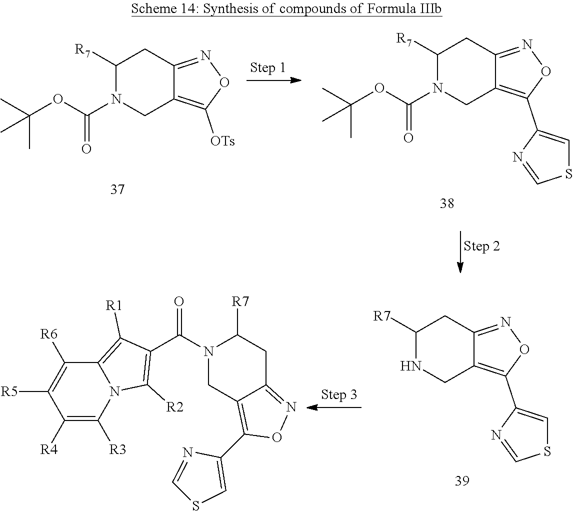

In Step 1 in Scheme 14 a transition metal catalysed cross coupling reaction on compound 37 (drawn as but not limited to a tosylate) with, for example 4-(tributylstannyl)-1,3-thiazole gives a compound of general structure 38 (WO2018/124060). The nitrogen protective group of compound 38 in Scheme 14 is in step 2 deprotected (WO2018/011162, A. Isidro-Llobet et al., Chem. Rev., 2009, 109, 2455-2504), drawn as but not limited to Boc, e.g. with HCl to give an amine of general structure 39. An amide coupling in step 3 with methods known in literature (A. El-Faham, F. Albericio, Chem. Rev. 2011, 111, 6557-6602), e.g. with HATU results in compounds of Formula IIIb.

In a further embodiment, compounds of Formula Mc can be prepared as shown in S.

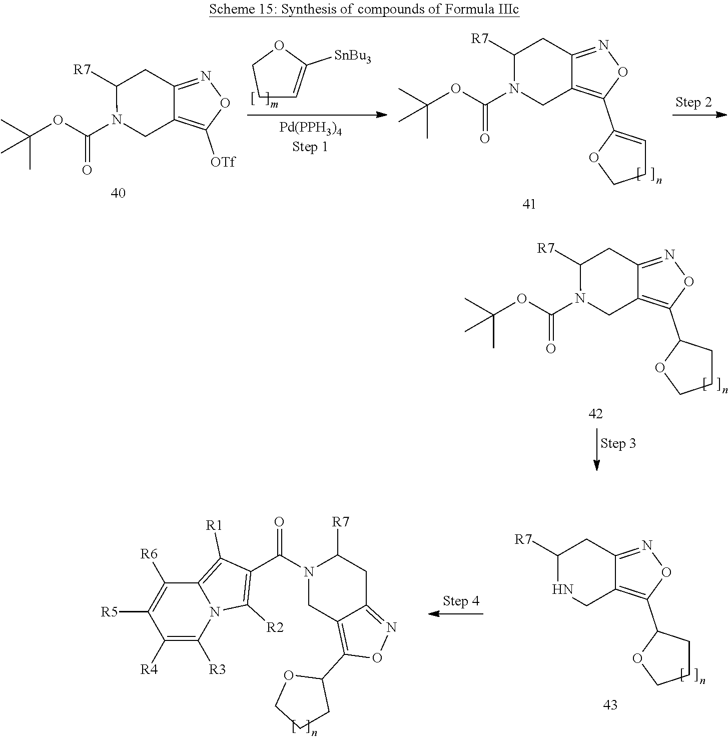

Compound 40 described in Scheme 15 (drawn as but not limited to a triflate) is in step 1 coupled with a organo-metallate (drawn as, but not limited to a dihydrofuran-2-yl tributyl tin) under palladium catalysis e.g. with Pd(PPh₃)₄ to give compounds of general structure 41. Reduction of the double bond e.g. with H₂ and palladium on carbon gives compounds of general structure 42. The nitrogen protective group of 42 in Scheme 15 is in step 3 deprotected (WO2018/011162, A. Isidro-Llobet et al., Chem. Rev., 2009, 109, 2455-2504), drawn as but not limited to Boc, e.g. with HCl to give an amine of general structure 43. An amide coupling in step 4 with methods known in literature (A. El-Faham, F. Albericio, Chem. Rev. 2011, 111, 6557-6602), e.g. with HATU results in compounds of Formula IIIc.

In a further embodiment, compounds of Formula IVb can be prepared as shown in Scheme 16.

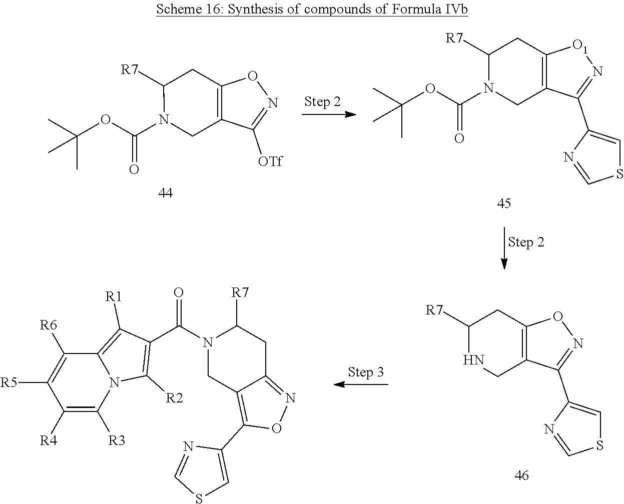

In Step 1 in Scheme 16 a transition metal catalysed cross coupling reaction on compound 44 (drawn as but not limited to a triflate) with, for example 4-(tributylstannyl)-1,3-thiazole gives a compound of general structure 45 (WO2018/124060). The nitrogen protective group of compound 45 in Scheme 16 is in step 2 deprotected (WO2018/011162, A. Isidro-Llobet et al., Chem. Rev., 2009, 109, 2455-2504), drawn as but not limited to Boc, e.g. with HCl to give an amine of general structure 46. An amide coupling in step 3 with methods known in literature (A. El-Faham, F. Albericio, Chem. Rev. 2011, 111, 6557-6602), e.g. with HATU results in compounds of Formula IVb.

In a further embodiment, compounds of Formula IVc can be prepared as shown in Scheme 17.

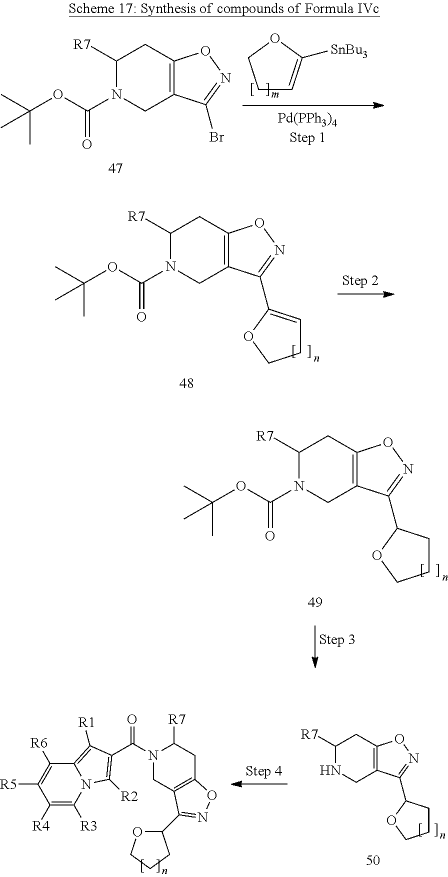

Compound 47 described in Scheme 17 (drawn as but not limited to a bromo substituted aromatic) is in step 1 coupled with a organo-metallate (drawn as, but not limited to a dihydrofuran-2-yl tributyl tin) under palladium catalysis e.g. with Pd(PPh₃)₄ to give compounds of general structure 48. Reduction of the double bond e.g. with H₂ and palladium on carbon gives compounds of general structure 49. The nitrogen protective group of 49 in Scheme 17 is in step 3 deprotected (WO2018/011162, A. Isidro-Llobet et al., Chem. Rev., 2009, 109, 2455-2504), drawn as but not limited to Boc, e.g. with HCl to give an amine of general structure 50. An amide coupling in step 4 with methods known in literature (A. El-Faham, F. Albericio, Chem. Rev. 2011, 111, 6557-6602), e.g. with HATU results in compounds of Formula IVc.

In a further embodiment, compounds of Formula Vb can be prepared as shown in Scheme 18.

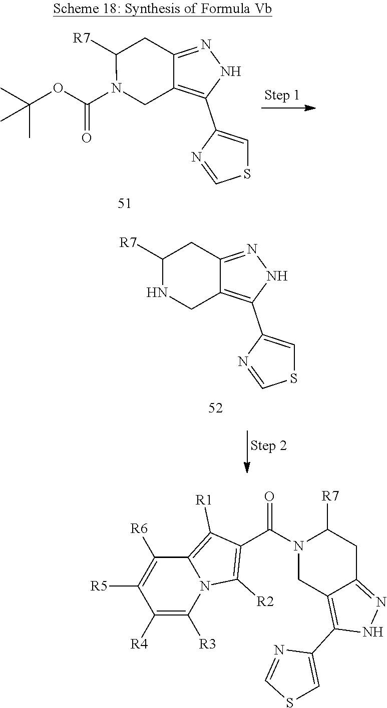

In Step 1 in Scheme 18 the nitrogen protective group of compound 51 is removed (WO2018/011162, A. Isidro-Llobet et al., Chem. Rev., 2009, 109, 2455-2504), drawn as but not limited to Boc, e.g. with HCl to give an amine of general structure 52. An amide coupling in step 2 with methods known in literature (A. El-Faham, F. Albericio, Chem. Rev. 2011, 111, 6557-6602), e.g. with HATU results in compounds of Formula Vb.

In a further embodiment, compounds of Formula Vc can be prepared as shown in Scheme 19

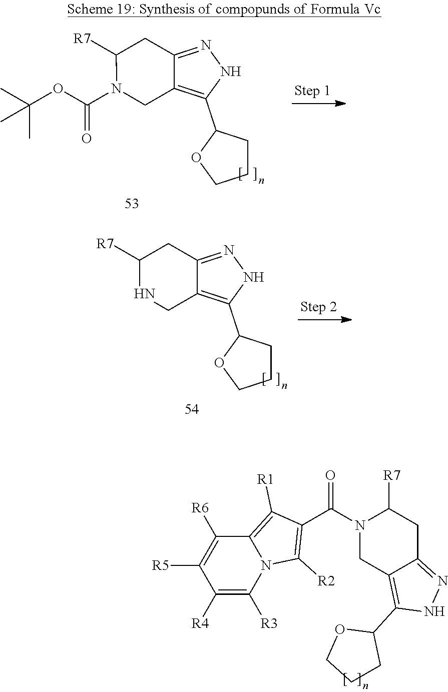

The nitrogen protective group of 53 in Scheme 19 is in step 1 deprotected (WO2018/011162, A. Isidro-Llobet et al., Chem. Rev., 2009, 109, 2455-2504), drawn as but not limited to Boc, e.g. with HCl to give an amine of general structure 54. An amide coupling in step 2 with methods known in literature (A. El-Faham, F. Albericio, Chem. Rev. 2011, 111, 6557-6602), e.g. with HATU results in compounds of Formula Vc.

The following examples illustrate the preparation and properties of some specific compounds of the invention.

The following abbreviations are used:

A—DNA nucleobase adenine

ACN—acetonitrile

Ar—argon

BODIPY-FL—4,4-difluoro-5,7-dimethyl-4-bora-3a,4a-diaza-s-indacene-3-propionic acid

(fluorescent dye)

Boc—tert-butoxycarbonyl

BnOH—benzyl alcohol

n-BuLi—n-butyl lithium

t-BuLi—t-butyl lithium

C—DNA nucleobase cytosine

CC₅₀—half-maximal cytotoxic concentration

CO₂—carbon dioxide

CuCN—copper (I) cyanide

DABCO—1,4-diazabicyclo[2.2.2]octane

DCE—dichloroethane

DCM—dichloromethane

Dess-Martin periodinane—1,1,1-triacetoxy-1,1-dihydro-1,2-benziodoxol-3(1H)-one

DIPEA—diisopropylethylamine

DIPE—di-isopropyl ether

DMAP—4-dimethylaminopyridine

DMF—N,N-dimethylformamide

DMP—Dess-Martin periodinane

DMSO—dimethyl sulfoxide

DNA—deoxyribonucleic acid

DPPA—diphenylphosphoryl azide

DTT—dithiothreitol

EC₅₀—half-maximal effective concentration

EDCI—N-(3-dimethylaminopropyl)-N′-ethylcarbodiimide hydrochloride

Et₂O—diethyl ether

EtOAc—ethyl acetate

EtOH—ethanol

FL-—five prime end labled with fluorescein

NEt₃—triethylamine

ELS—Evaporative Light Scattering

g—gram(s)

G—DNA nucleobase guanine

HBV—hepatitis B virus

HATU—2-(1H-7-azabenzotriazol-1-yl)-1,1,3,3-tetramethyl uronium hexafluorophosphate

HCl—hydrochloric acid

HEPES—4-(2-hydroxyethyl)-1-piperazineethanesulfonic acid

HOAt—1-hydroxy-7-azabenzotriazole

HOBt—1-hydroxybenzotriazole

HPLC—high performance liquid chromatography

IC₅₀—half-maximal inhibitory concentration

LC640-—3 prime end modification with fluorescent dye LightCycler® Red 640

LC/MS—liquid chromatography/mass spectrometry

LiAlH₄—lithium aluminium hydride

LiOH—lithium hydroxide

MeOH—methanol

MeCN—acetonitrile

MgSO₄—magnesium sulfate

mg—milligram(s)

min—minutes

mol—moles

mmol—millimole(s)

mL—millilitre(s)

MTBE—methyl tert-butyl ether

N₂—nitrogen

Na₂CO₃—sodium carbonate

NaHCO₃—sodium hydrogen carbonate

Na₂SO₄—sodium sulfate

NdeI—restriction enzyme recognizes CAATATG sites

NEt₃—triethylamine

NaH—sodium hydride

NaOH—sodium hydroxide

NH₃—ammonia

NH₄Cl—ammonium chloride

¹H NMR—nuclear magnetic resonance

PAGE—polyacrylamide gel electrophoresis

PCR—polymerase chain reaction

qPCR—quantitative PCR

Pd/C—palladium on carbon

-PH— 3 prime end phosphate modification

pTSA—4-toluene-sulfonic acid

Rt—retention time

r.t. —room temperature

sat. —saturated aqueous solution

SDS—sodium dodecyl sulfate

SI—selectivity index (═CC₅₀/EC₅₀)

STAB—sodium triacetoxyborohydride

T—DNA nucleobase thymine

TBAF—tetrabutylammonium fluoride

TEA—triethylamine

TFA—trifluoroacetic acid

THF—tetrahydrofuran

TLC—thin layer chromatography

Tris—tris(hydroxymethyl)-aminomethane

XhoI—restriction enzyme recognizes CATCGAG sites

Compound Identification—NMR

For a number of compounds, NMR spectra were recorded either using a Bruker DPX400 spectrometer equipped with a 5 mm reverse triple-resonance probe head operating at 400 MHz for the proton and 100 MHz for carbon, or using a Bruker DRX500 spectrometer equipped with a 5 mm reverse triple-resonance probe head operating at 500 MHz for the proton and 125 MHz for carbon. Deuterated solvents were chloroform-d (deuterated chloroform, CDCl₃) or d6-DMSO (deuterated DMSO, d6-dimethylsulfoxide). Chemical shifts are reported in parts per million (ppm) relative to tetramethylsilane (TMS) which was used as internal standard.

Compound Identification—HPLC/MS

For a number of compounds, LC-MS spectra were recorded using the following analytical methods.

Method A

Column—Reverse phase Waters Xselect CSH C18 (50×2.1 mm, 3.5 micron)

Flow—0.8 mL/min, 25 degrees Celsius

Eluent A—95% acetonitrile+5% 10 mM ammonium carbonate in water (pH 9)

Eluent B—10 mM ammonium carbonate in water (pH 9)

Linear gradient t=0 min 5% A, t=3.5 min 98% A. t=6 min 98% A

Method A2

Column—Reverse phase Waters Xselect CSH C18 (50×2.1 mm, 3.5 micron)

Flow—0.8 mL/min, 25 degrees Celsius

Eluent A—95% acetonitrile+5% 10 mM ammonium carbonate in water (pH 9)

Eluent B—10 mM ammonium carbonate in water (pH 9)

Linear gradient t=0 min 5% A, t=4.5 min 98% A. t=6 min 98% A

Method B

Column—Reverse phase Waters Xselect CSH C18 (50×2.1 mm, 3.5 micron)

Flow—0.8 mL/min, 35 degrees Celsius

Eluent A—0.1% formic acid in acetonitrile

Eluent B—0.1% formic acid in water

Linear gradient t=0 min 5% A, t=3.5 min 98% A. t=6 min 98% A

Method B2

Column—Reverse phase Waters Xselect CSH C18 (50×2.1 mm, 3.5 micron)

Flow—0.8 mL/min, 40 degrees Celsius

Eluent A—0.1% formic acid in acetonitrile

Eluent B—0.1% formic acid in water

Linear gradient t=0 min 5% A, t=4.5 min 98% A. t=6 min 98% A

Method C

Column—Reverse phase Waters Xselect CSH C18 (50×2.1 mm, 3.5 micron)

Flow—1 mL/min, 35 degrees Celsius

Eluent A—0.1% formic acid in acetonitrile

Eluent B—0.1% formic acid in water

Linear gradient t=0 min 5% A, t=1.6 min 98% A. t=3 min 98% A

Method D

Column—Phenomenex Gemini NX C18 (50×2.0 mm, 3.0 micron)

Flow—0.8 mL/min, 35 degrees Celsius

Eluent A—95% acetonitrile+5% 10 mM ammonium bicarbonate in water

Eluent B—10 mM ammonium bicarbonate in water pH=9.0

Linear gradient t=0 min 5% A, t=3.5 min 98% A. t=6 min 98% A

Method E

Column—Phenomenex Gemini NX C18 (50×2.0 mm, 3.0 micron)

Flow—0.8 mL/min, 25 degrees Celsius

Eluent A—95% acetonitrile+5% 10 mM ammonium bicarbonate in water

Eluent B—10 mM ammonium bicarbonate in water (pH 9)

Linear gradient t=0 min 5% A, t=3.5 min 30% A. t=7 min 98% A, t=10 min 98% A

Method F

Column—Waters XSelect HSS C18 (150×4.6 mm, 3.5 micron)

Flow—1.0 mL/min, 25 degrees Celsius

Eluent A—0.1% TFA in acetonitrile

Eluent B—0.1% TFA in water

Linear gradient t=0 min 2% A, t=1 min 2% A, t=15 min 60% A, t=20 min 60% A

Method G

Column—Zorbax SB-C18 1.8 μm 4.6×15 mm Rapid Resolution cartridge (PN 821975-932)

Flow—3 mL/min

Eluent A—0.1% formic acid in acetonitrile

Eluent B—0.1% formic acid in water

Linear gradient t=0 min 0% A, t=1.8 min 100% A

Method H

Column—Waters Xselect CSH C18 (50×2.1 mm, 2.5 micron)

Flow—0.6 mL/min

Eluent A—0.1% formic acid in acetonitrile

Eluent B—0.1% formic acid in water

Linear gradient t=0 min 5% A, t=2.0 min 98% A, t=2.7 min 98% A

Method J

Column—Reverse phase Waters Xselect CSH C18 (50×2.1 mm, 2.5 micron)

Flow—0.6 mL/min

Eluent A—100% acetonitrile

Eluent B—10 mM ammonium bicarbonate in water (pH 7.9)

Linear gradient t=0 min 5% A, t=2.0 min 98% A, t=2.7 min 98% A

### Preparation of 5-[(tert-butoxy)carbonyl]-6-methyl-1-{[2-(trimethylsilyl)ethoxy]methyl}-1H,4H,5H,6H,7H-pyrazolo[4,3-c]pyridine-3-carboxylic Acid

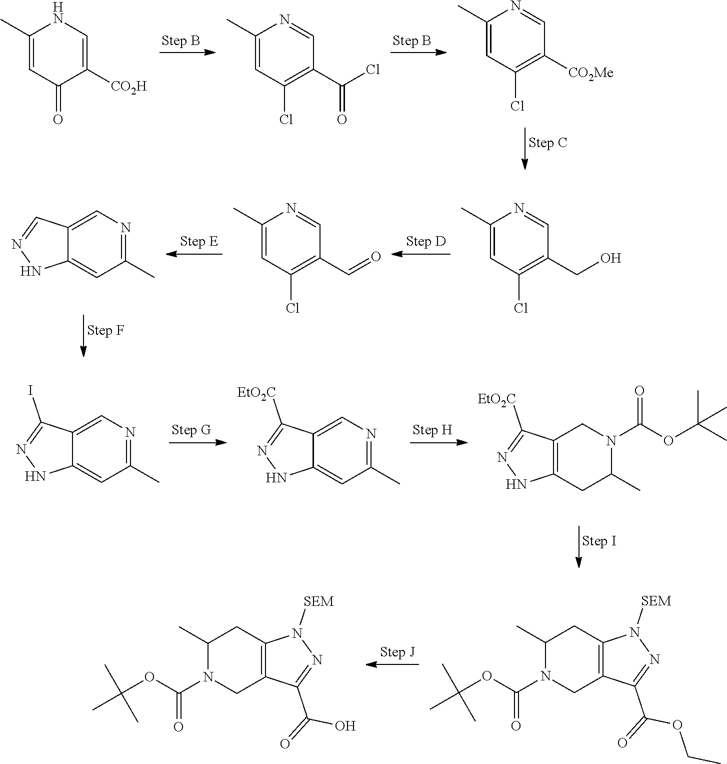

### Step A

6-Methyl-4-oxo-1,4-dihydropyridine-3-carboxylic acid (50.0 g, 326.51 mmol) was suspended in phosphoryl trichloride (500.0 g, 3.26 mol) and stirred at 95° C. for 16 h. After cooling, the excess phosphorus oxychloride was distilled off in vacuo, and obtained residue was evaporated with toluene (2×250 mL) to give 5-(carboxy)-4-chloro-2-methylpyridin-1-ium chloride (73.3 g, 95.0% purity, 307.46 mmol, 94.2% yield).

### Step B

5-(Carboxy)-4-chloro-2-methylpyridin-1-ium chloride (73.3 g, 323.64 mmol) was dissolved in THF (500 mL) and MeOH (500 mL) was added dropwise at 100° C. The mixture was stirred at r.t. for 2 h. The mixture was concentrated to give a residue which was dissolved in CH₂Cl₂ (700 mL) and washed with a saturated solution of NaHCO₃. The combined organic extracts were concentrated in vacuo to give an orange oil which was purified by column chromatography (MTBE-hexane 2:1) (Rf=0.8) to yield methyl 4-chloro-6-methylpyridine-3-carboxylate (57.7 g, 98.0% purity, 304.65 mmol, 94.1% yield) as a yellow oil that crystallized on standing to give a the yellow solid.

### Step C

To a cooled (−25° C.) suspension of lithium aluminium hydride (6 g) in THF (500 mL) was added dropwise a solution of methyl 4-chloro-6-methylnicotinate (33.0 g, 177.79 mmol) in tetrahydrofuran (100 mL). The mixture was stirred at 0° C. for 1.5 hours. Water (6 mL in 50 mL of THF), 15% aqueous solution of sodium hydroxide (6 mL) and water (18 mL) were dropped successively to the reaction mixture. The mixture was stirred at r.t. for 30 minutes, filtered and the filter cake washed with THF (2×200 mL). The filtrate was concentrated to give the title compound (4-chloro-6-methylpyridin-3-yl)methanol (26.3 g, 95.0% purity, 158.54 mmol, 89.2% yield) as an yellow solid that was used without further purification.

### Step D

To a solution of (4-chloro-6-methylpyridin-3-yl)methanol (26.3 g, 166.88 mmol) in DCM (777 mL) was added 1,1,1-tris(acetoxy)-1,1-dihydro-1,2-benziodoxol-3(1H)-one (81.4 g, 191.92 mmol) in few portions, maintaining the temperature below 5° C. with an water/ice cooling bath. After the reaction was complete (monitored by 1H NMR) the mixture was poured into a stirred aqueous solution of sodium hydrogen carbonate (16.12 g, 191.91 mmol) and Na₂S₂O₃ and stirred until organic phase became transparent (about 2 h). The layers were separated and aqueous layer was extracted with DCM (3×300 mL), and the combined organic extracts were washed with brine, dried over Na₂SO₄ and concentrated under reduced pressure to give 4-chloro-6-methylpyridine-3-carbaldehyde (21.0 g, 90.0% purity, 121.48 mmol, 72.8% yield) that was used in the next step without further purification.

### Step E

To a suspension of 4-chloro-6-methylpyridine-3-carbaldehyde (17.0 g, 109.27 mmol) (1 equiv.) in ethylene glycol dimethyl ether (300 mL) and 1,4-dioxane (300 ml) was added hydrazine hydrate (191.45 g, 3.82 mol) (98 percent) (35.00 equiv.). The mixture was refluxed for 96 h NMR analysis). The layers were separated and the organic layer was concentrated under reduced pressure. Water (200 mL) was added to the residue, and the mixture was stirred at room temperature for 1 hour. Product was collected by filtration, washed with water (100 mL), then dried to give 6-methyl-1H-pyrazolo[4,3-c]pyridine (3.42 g, 98.0% purity, 25.17 mmol, 23% yield) as a yellow solid.

### Step F

A suspension of 6-methyl-1H-pyrazolo[4,3-c]pyridine (1.91 g, 14.34 mmol) (1.00 equiv), iodine (7.28 g, 28.69 mmol) (2.00 equiv), and potassium hydroxide (2.9 g, 51.63 mmol) (3.60 equiv) in DMF (40 mL) was stirred at r.t. for 12 h. The reaction was quenched by addition of saturated aqueous Na₂S₂O₃, extracted with ethyl acetate (3×200 mL), dried over anhydrous sodium sulfate and concentrated under reduced pressure to give 3-iodo-6-methyl-1H-pyrazolo[4,3-c]pyridine (3.1 g, 98.0% purity, 11.73 mmol, 81.8% yield) as a yellow solid.

### Step G

3-Iodo-6-methyl-1H-pyrazolo[4,3-c]pyridine (5.05 g, 19.49 mmol), triethylamine (2.37 g, 23.39 mmol, 3.26 ml) and Pd(dppf)Cl₂ (3 mol %) were dissolved in ethanol (96%, 200 ml). The reaction mixture was heated at 120° C. in high pressure vessel at 40 atm CO pressure for 18 h. The mixture was then concentrated and water (100 ml) was added to the obtained residue. The mixture was stirred at room temperature for 1 hour and product collected by filtration. The solid was washed with water (100 mL), then dried to give ethyl 6-methyl-1H-pyrazolo[4,3-c]pyridine-3-carboxylate (2.7 g, 95.0% purity, 12.5 mmol, 64.1% yield) as an orange solid.

### Step H

To a suspension of ethyl 6-methyl-1H-pyrazolo[4,3-c]pyridine-3-carboxylate (620.23 mg, 3.02 mmol) and di-tert-butyl dicarbonate (692.6 mg, 3.17 mmol) in methanol (133 mL) (plus 5 drops of Et₃N) was added 20% Pd(OH)₂ on carbon. The mixture was hydrogenated in an autoclave at 40 bar and then allowed to stir at r.t for 18 h. The reaction mixture was filtered through a thin pad of silica and the pad was washed with CH₃OH (30 mL). The filtrate was concentrated under reduced pressure to give 5-tert-butyl 3-ethyl 6-methyl-1H,4H,5H,6H,7H-pyrazolo[4,3-c]pyridine-3,5-dicarboxylate (888.89 mg, 98.0% purity, 2.82 mmol, 93.2% yield) as an oil.

### Step I

To a cooled (0° C.) solution of 5-tert-butyl 3-ethyl 6-methyl-1H,4H,5H,6H,7H-pyrazolo[4,3-c]pyridine-3,5-dicarboxylate (1.1 g, 3.56 mmol) (1 eq.) in THF (75 ml) was added sodium hydride (60%, 1.33 eq) portionwise. The mixture was stirred at room temperature for 0.5 h. [2-(Chloromethoxy)ethyl]trimethylsilane (788.36 mg, 4.73 mmol) was added dropwise and the mixture stirred at room temperature for an additional 16 h. The mixture was quenched with water and extracted with EtOAc (3×30 mL). The combined organic extracts were dried over anhydrous Na₂SO₄, filtered and concentrated under reduced pressure to give 5-tert-butyl 3-ethyl 6-methyl-1-[2-(trimethylsilyl)ethoxy]methyl-1H,4H, 5H,6H,7H-pyrazolo[4,3-c]pyridine-3,5-dicarboxylate (1.56 g, 64.0% purity, 2.26 mmol, 63.7% yield) as yellow oil that was used in the next step without further purification.

### Step J

5-Tert-butyl 3-ethyl 6-methyl-1-[2-(trimethylsilyl)ethoxy]methyl-1H,4H,5H,6H,7H-pyrazolo[4,3-c]pyridine-3,5-dicarboxylate (808.0 mg, 1.84 mmol) and lithium hydroxide monohydrate (231.25 mg, 5.51 mmol) were stirred in a mixture of THF:H₂O:CH₃OH (v/v 3:1:1, 50 mL) at 25° C. for 18 h. The reaction mixture was then concentrated under reduced pressure and acidified to pH 4 with a saturated aqueous solution of citric acid. The mixture was extracted with EtOAc (3×30 mL). The combined organic extracts were dried over anhydrous Na₂SO₄, filtered and concentrated under reduced pressure. The crude product was purified by HPLC to give 5-[(tert-butoxy)carbonyl]-6-methyl-1-[2-(trimethylsilyl)ethoxy]methyl-1H,4H,5H,6H,7H-pyrazolo[4,3-c]pyridine-3-carboxylic acid (505.0 mg, 99.0% purity, 1.21 mmol, 66.1% yield) as white solid.

Rt (Method G) 1.57 mins, m/z 412 [M+H]⁺

¹H NMR (400 MHz, DMSO) δ −0.07 (s, 9H), 0.80 (t, J=7.9 Hz, 2H), 1.02 (d, J=6.9 Hz, 3H), 1.41 (s, 9H), 2.69 (d, J=16.4 Hz, 1H), 2.83 (dd, J=16.3, 6.1 Hz, 1H), 3.48 (m, 2H), 3.98 (d, J=17.5 Hz, 1H), 4.71 (br.s, 1H), 4.88 (d, J=17.1 Hz, 1H), 5.39 (AB-system, 2H), 12.77 (br.s, 1H).

### Preparation of 5-[(tert-butoxy)carbonyl]-1-{[2-(trimethylsilyl)ethoxy]methyl}-1H,4H,5H,6H,7H-pyrazolo[4,3-c]pyridine-3-carboxylic Acid

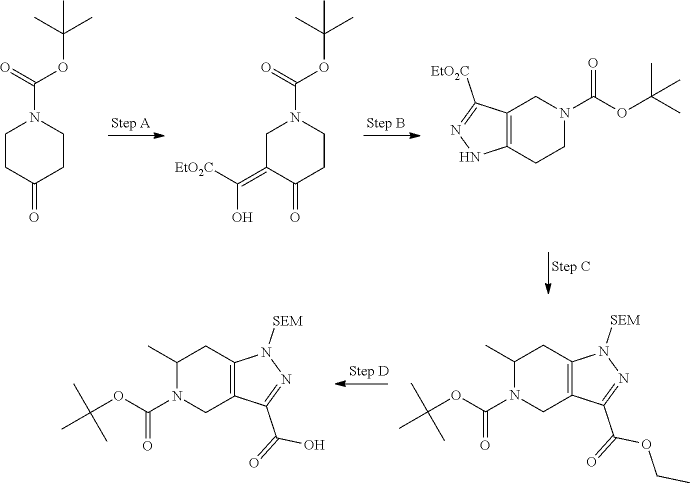 [SMILES: CC(C)(C)OC(=O)N1CCC(=O)CC1 ; CCOC(=O)/C(O)=C1\CN(C(=O)OC(C)(C)C)CCC1=O ; CCOC(=O)c1n[nH]c2c1CN(C(=O)OC(C)(C)C)CC2 ; CC1Cc2c(c(C(=O)O)nn2COCC[Si](C)(C)C)CN1C(=O)OC(C)(C)C ; CCOC(=O)c1nn(COCC[Si](C)(C)C)c2c1CN(C(=O)OC(C)(C)C)C(C)C2]

### Step A

Lithium bis(trimethylsilyl)amide (8.4 g, 50.21 mmol, 50.21 ml) was dissolved in dry Et₂O (50 mL) and cooled to −78° C. (dry-ice/acetone). To the cooled mixture was added a solution of tert-butyl 4-oxopiperidine-1-carboxylate (10.0 g, 50.21 mmol) in dry Et₂O/THF (3:1) (60 mL).Once addition was complete, the mixture was stirred for 30 min A solution of diethyl oxalate (7.34 g, 50.21 mmol, 6.82 ml) in dry Et₂O (20 mL) was added over 10 min. The mixture was stirred for 15 min at −78° C. after which the cooling was removed. The reaction mixture was stirred overnight at 20° C. The mixture was poured into 1M KHSO₄ (200 mL) and the layers were separated. The aqueous phase was extracted with EtOAc (2×100 mL). The combined organic layers were separated, washed with water, dried (Na₂SO₄), filtered and concentrated to give tert-butyl 3-(2-ethoxy-2-oxoacetyl)-4-oxopiperidine-1-carboxylate (14.1 g, 47.11 mmol, 93.8% yield) as orange oil, which was used in the next step without further purification.

### Step B

To a stirred solution of tert-butyl 3-(2-ethoxy-2-oxoacetyl)-4-oxopiperidine-1-carboxylate (14.11 g, 47.14 mmol) in abs. EtOH (150 mL) was added acetic acid (4.53 g, 75.43 mmol, 4.32 ml) followed by portionwise addition of hydrazine hydrate (2.36 g, 47.14 mmol, 3.93 ml) The mixture was stirred for 5 h, then concentrated, and the residue obtained diluted with sat. NaHCO₃. The product was extracted with EtOAc (2×100 mL). The combined organic extracts were dried over Na₂SO₄, filtered and concentrated to afford 5-tert-butyl 3-ethyl 1H,4H,5H,6H,7H-pyrazolo[4,3-c]pyridine-3,5-dicarboxylate (11.2 g, 37.92 mmol, 80.4% yield) as yellow foam, crystallized with standing.

### Step C

To a cooled (0° C.) suspension of sodium hydride (1.82 g, 0.045 mol, 60% dispersion in mineral oil) in dry THF (250 mL) under argon was added dropwise a solution of 5-tert-butyl 3-ethyl 1H,4H,5H,6H,7H-pyrazolo[4,3-c]pyridine-3,5-dicarboxylate (11.2 g, 37.92 mmol) in dry THF (50 mL). The mixture was stirred for 30 min at 0° C., then [2-(chloromethoxy)ethyl]trimethylsilane (7.59 g, 45.51 mmol) was added dropwise. The resulting mixture was stirred for 30 min at 0° C., and then warmed to room temperature. The mixture was poured in water (250 mL), and the product was extracted with EtOAc (2×200 mL). The combined organic extracts were washed with brine, dried over Na₂SO₄ and concentrated to afford crude 5-tert-butyl 3-ethyl 1-[2-(trimethylsilyl)ethoxyl methyl-1H,4H,5H, 6H,7H-pyrazolo[4,3-c]pyridine-3,5-dicarboxylate (15.3 g, 35.95 mmol, 94.8% yield) as yellow oil which was used in the next step without further purification.

### Step D

To a solution of 5-tert-butyl 3-ethyl 1-[2-(trimethylsilyl)ethoxy]methyl-1H,4H,5H,6H,7H-pyrazolo[4,3-c]pyridine-3,5-dicarboxylate (15.3 g, 35.95 mmol) in THF (100 mL)/water (50 mL) was added lithium hydroxide monohydrate (5.28 g, 125.82 mmol). The reaction mixture was stirred at 50° C. for 3 h, and then concentrated. The residue was carefully acidified with sat. aq. solution of KHSO₄ to pH 4-5 and product was extracted with EtOAc (2×200 mL). The combined organic extracts were dried with Na₂SO₄, filtered and evaporated. The solid residue was triturated with hexane. Product was collected by filtration and dried to afford 5-[(tert-butoxy)carbonyl]-1-[2-(trimethylsilyl)ethoxy]methyl-1H,4H,5H,6H,7H-pyrazolo[4,3-c]pyridine-3-carboxylic acid (7.5 g, 18.87 mmol, 52.5% yield) as yellow solid.

Rt (Method G) 1.52 mins, m/z 398 [M+H]⁺

1H NMR (400 MHz, CDCl₃) 15-0.05 (s, 9H), 0.87 (t, J=8.2 Hz, 2H), 1.47 (s, 9H), 2.78 (m, 2H), 3.55 (m, 2H), 3.71 (m, 2H), 4.62 (br.s, 2H), 5.43 (s, 2H), COOH is not observed.

### Preparation of 6,6-difluoro-4-azaspiro[2.4]heptane

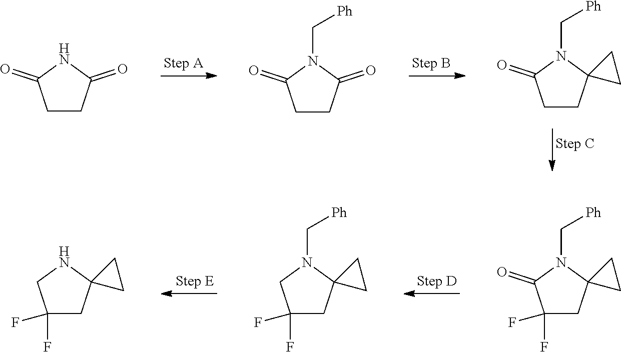 [SMILES: O=C1CCC(=O)N1 ; O=C1CCC(=O)N1Cc1ccccc1 ; O=C1CCC2(CC2)N1Cc1ccccc1 ; FC1(F)CNC2(CC2)C1 ; FC1(F)CN(Cc2ccccc2)C2(CC2)C1 ; O=C1N(Cc2ccccc2)C2(CC2)CC1(F)F]

### Step A

To a solution of succinic anhydride (100 g, 1000 mmol) in toluene (3000 mL) was added benzylamine (107 g, 1000 mmol). The solution was stirred at room temperature for 24 h, and then heated at reflux with a DeanStark apparatus for 16 hours. The mixture was then concentrated under reduced pressure to give 1-benzylpyrrolidine-2,5-dione (170 g, 900 mmol, 90% yield).

### Step B

To a cooled (0° C.) mixture of 1-benzylpyrrolidine-2,5-dione (114 g, 600 mmol) and Ti(Oi-Pr)₄ (170.5 g, 600 mmol) in dry THF (2000 mL) under argon atmosphere was added dropwise a 3.4M solution of ethyl magnesium bromide in THF (1200 mmol). The mixture was warmed to room temperature and stirred for 4 h. BF₃.Et₂O (170 g, 1200 mmol) was then added dropwise and the solution stirred for 6 h. The mixture was cooled (0° C.) and 3N hydrochloric acid (500 mL) was added. The mixture was extracted twice with Et₂O, and the combined organic extracts washed with brine, dried and concentrated under reduced pressure to give 4-benzyl-4-azaspiro[2.4]heptan-5-one (30.2 g, 150 mmol, 25% yield).

### Step C

To a cooled (−78° C.) solution of 4-benzyl-4-azaspiro[2.4]heptan-5-one (34.2 g, 170 mmol) in dry THF (1000 mL) under argon was added LiHMDS in THF (1.1M solution, 240 mmol). The mixture was stirred for 1 h, and then a solution of N-fluorobenzenesulfonimide (75.7 g, 240 mmol) in THF (200 mL) was added dropwise. The mixture was warmed to room temperature and stirred for 6 h. The mixture was then re-cooled (−78° C.) and LiHMDS added (1.1M solution in THF, 240 mmol).

The solution was stirred for 1 h, and then N-fluorobenzenesulfonimide (75.7 g, 240 mmol) in THF (200 mL) was added dropwise. The mixture was warmed to room temperature and stirred for 6 h. The mixture was poured into a saturated solution of NH₄Cl (300 mL) and extracted twice with Et₂O. The combined organic extracts were washed with brine and concentrated under reduced pressure. Product was purified by column chromatography to provide 4-benzyl-6,6-difluoro-4-azaspiro[2.4]heptan-5-one (18 g, 75.9 mmol, 45% yield).

### Step D

To a warmed (40° C.) solution of BH₃.Me₂S (3.42 g, 45 mmol) in THF (200 mL) was added dropwise 4-benzyl-6,6-difluoro-4-azaspiro[2.4]heptan-5-one (11.9 g, 50 mmol). The mixture was stirred for 24 h at 40° C., and then cooled to room temperature. Water (50 mL) was added dropwise, and the mixture extracted with Et₂O (2×200 mL). The combined organic extracts were washed brine, diluted with 10% solution of HCl in dioxane (50 mL) and evaporated under reduced pressure to give 4-benzyl-6,6-difluoro-4-azaspiro[2.4]heptane (3 g, 13.4 mmol, 27% yield).

### Step E

4-benzyl-6,6-difluoro-4-azaspiro[2.4]heptane (2.68 g, 12 mmol) and palladium hydroxide (0.5 g) in methanol (500 mL) were stirred at room temperature under an atmosphere of H₂ for 24 h. The mixture was filtered and then filtrate concentrated under reduced pressure to obtain 6,6-difluoro-4-azaspiro[2.4]heptane (0.8 g, 6.01 mmol, 50% yield).

### Preparation of 6,6-difluoro-4-{2H,4H,5H,6H,7H-pyrazolo[4,3-c]pyridine-3-carbonyl}-4-azaspiro[2.4]heptane

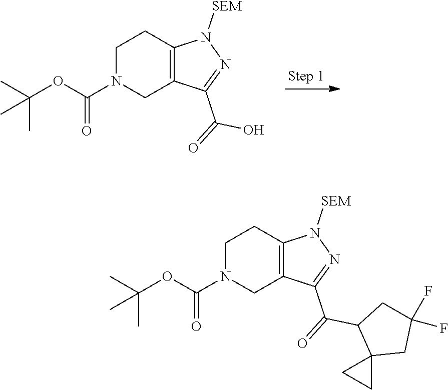 [SMILES: CC(C)(C)OC(=O)N1CCc2c(c(C(=O)O)nn2COCC[Si](C)(C)C)C1 ; CC(C)(C)OC(=O)N1CCc2c(c(C(=O)C3CC(F)(F)CC34CC4)nn2COCC[Si](C)(C)C)C1]

### Step 1

HATU (0.383 g, 1.006 mmol) was added to a solution of 5-(tertbutoxycarbonyl)-2-((2-(trimethylsilyl)ethoxy)methyl)-4,5,6,7-tetrahydro-2H-pyrazolo[4,3-c]pyridine-3-carboxylic acid (0.400 g, 1.006 mmol) in dry N,N-dimethylformamide (4 ml). DIPEA (0.527 ml, 3.02 mmol) and 6,6-difluoro-4-azaspiro[2.4]heptane hydrochloride (0.171 g, 1.006 mmol) were added. The mixture was stirred at r.t. for 5 days. The mixture was then poured into brine and extracted with ethyl acetate. The organic layer was separated, concentrated and purified by flash chromatography to give the desired product as a colourless oil (0.298 g, 58% yield).

LC-MS: m/z 513 (M+H)±

### Synthesis of 1-[(difluoromethoxy)methyl]-N-methylcyclopropan-1-amine

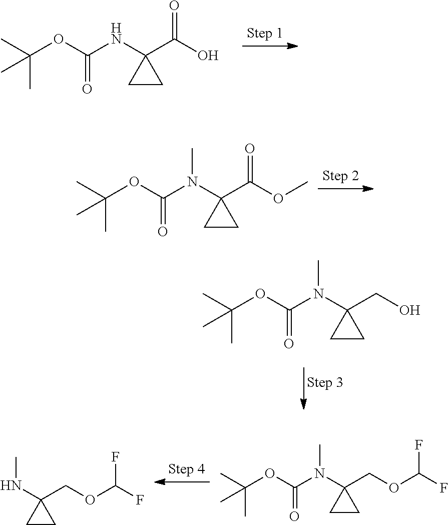

### Step 1

Sodium hydride (0.596 g, 14.91 mmol) was added to a cooled (0° C.) solution of 1-((tertbutoxycarbonyl)amino)cyclopropane-1-carboxylic acid (1 g, 4.97 mmol) in dry N,N-dimethylformamide (15 ml). When gas evolution had ceased, iodomethane (0.932 ml, 14.91 mmol) was added. The cooling bath was removed and the mixture was stirred for 2 h. The mixture was then cooled to 0° C. and quenched by addition of water. The mixture was partitioned between water and ethyl acetate, the organic layer was washed with brine, concentrated and purified by flash chromatography (24 g silica gel), flowrate 30 ml/min, 15 to 50% ethyl acetate in heptane over 15 min to give the desired product as a colorless oil (1.056 g, 93% yield).

### Step 2

To a solution of methyl 1-((tertbutoxycarbonyl)(methyl)amino)cyclopropane-1-carboxylate (1.05 g, 4.58 mmol) in dry THF (5 ml) under Na was added lithium borohydride (1.259 mL, 4M in THF, 5.04 mmol). The mixture was stirred at r.t. for 4 days. Sodium sulfate and water were added, the mixture was filtered over a pad of sodium sulfate which was rinsed with dichloromethane. The filtrate was concentrated, to give tert-butyl (1-(hydroxymethyl)cyclopropyl)(methyl)carbamate as a white solid (0.904 g, 95% yield).

### Step 3

To a solution of tert-butyl (1-(hydroxymethyl)cyclopropyl)(methyl)carbamate (0.100 g, 0.497 mmol) and (bromodifluoromethyl)trimethylsilane (0.155 ml, 0.994 mmol) in dichloromethane (0.5 ml) was added one drop of a solution of potassium acetate (0.195 g, 1.987 mmol) in water (0.5 ml). The mixture was stirred for 40 h. The mixture was diluted with dichloromethane and water, the organic layer was separated and concentrated. Purification by flash chromatography (20% ethyl acetate in heptane) gave tert-butyl N-{1[(difluoromethoxy)methyl]cyclopropyl}-N-methylcarbamate as colorless oil (0.058 g, 46% yield)

### Step 4

To tert-butyl (1-((difluoromethoxy)methyl)cyclopropyl)(methyl)carbamate (0.058 g, 0.231 mmol) was added HCl in dioxane (4M solution, 2 mL, 8.00 mmol). The mixture was stirred for 30 min at rt, then concentrated to yield the desired product which was used without further purification

LC-MS: m/z 152.2 (M+H)+

### Synthesis of tert-butyl 3-({1-[(difluoromethoxy)methyl]cyclopropyl}(methyl)carbamoyl)-4H,5H,6H,7H-pyrazolo[1,5-a]pyrazine-5-carboxylate

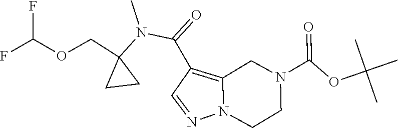 [SMILES: CN(C(=O)c1cnn2c1CN(C(=O)OC(C)(C)C)CC2)C1(COC(F)F)CC1]

To a solution of 5-(tert-butoxycarbonyl)-4,5,6,7-tetrahydropyrazolo[1,5-a]pyrazine-3-carboxylic acid (350 mg, 1.311 mmol) in dry N,N-dimethylformamide (3 ml) was added HATU (548 mg, 1.442 mmol). The mixture was stirred for 10 min. In a separate flask, 1-((difluoromethoxy)methyl)-N-methylcyclopropan-1-amine hydrochloride (246 mg, 1.311 mmol) was dissolved in dry N,N-dimethylformamide (3 ml) and triethylamine (0.914 ml, 6.56 mmol) was added. The two mixtures were combined and stirred for 1 h. The reaction mixture was partitioned between water (50 mL) and EtOAc (50 mL). The layers were separated and the aqueous layer was extracted with 50 ml EtOAc. The combined organic layers were washed with 4×50 ml brine, dried with Na₂SO₄ and concentrated. The product was dissolved in 3 ml DCM and purified by straight phase column chromatography, but no separation was observed between the desired product and the major by-product (0.462 g, 87% purity, 88% yield) The material was used in the next step without further purification.

### Synthesis of tert-butyl 3-({11-[(difluoromethoxy)methyl]cyclopropyl}(methyl)carbamoyl)-6-methyl-4H,5H,6H,7H-pyrazolo[1,5-a]pyrazine-5-carboxylate

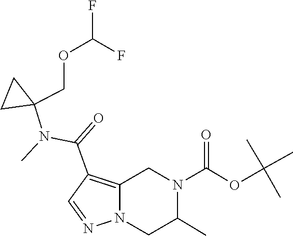 [SMILES: CC1Cn2ncc(C(=O)N(C)C3(COC(F)F)CC3)c2CN1C(=O)OC(C)(C)C]

To a solution of 5-(tert-butoxycarbonyl)-6-methyl-4,5,6,7-tetrahydropyrazolo[1,5-a]pyrazine-3-carboxylic acid (138 mg, 0.490 mmol) in dry N,N-dimethylformamide (1.6 ml) was added HATU (205 mg, 0.539 mmol). The mixture was stirred for 10 min. In a separate flask, 1-((difluoromethoxy)methyl)-N-methylcyclopropan-1-amine hydrochloride (92 mg, 0.490 mmol) was dissolved in dry N,N-dimethylformamide (1.1 ml) and triethylamine (0.342 ml, 2.452 mmol) was added. The two mixtures were combined and stirred for 1 h. The reaction mixture was partitioned between water (15 mL) and EtOAc (15 mL). The layers were separated and the aqueous layer was extracted with EtOAc (15 mL). The combined organic extracts were washed with brine (4×15 mL), dried with Na₂SO₄ and concentrated. The residue was dissolved in 1 ml DCM and purified by straight phase column chromatography to give the desired product (0.163 g, 80% yield, 81% purity).

### Synthesis of N-{1-[(difluoromethoxy)methyl]cyclopropyl}-N-methyl-4H,5H,6H,7H-pyrazolo[1,5-a]pyrazine-3-carboxamide

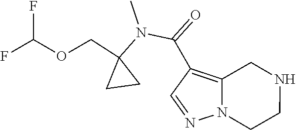 [SMILES: CN(C(=O)c1cnn2c1CNCC2)C1(COC(F)F)CC1]

Tert-butyl 3-((1-((difluoromethoxy)methyl)cyclopropyl)(methyl)carbamoyl)-6,7-dihydropyrazolo[1,5-a]pyrazine-5(4H)-carboxylate (0.284 g, 0.71 mmol) was dissolved in HCl (4M in dioxane) (2 ml, 8.00 mmol) and the mixture was stirred for 1 h. The reaction mixture was concentrated to give the desired product which was used without further purification.

### Synthesis of N-{1-[(difluoromethoxy)methyl]cyclopropyl}-N,6-dimethyl-4H,5H,6H,7H-pyrazolo[1,5-a]pyrazine-3-carboxamide

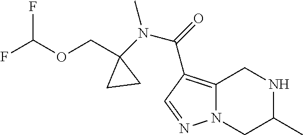 [SMILES: CC1Cn2ncc(C(=O)N(C)C3(COC(F)F)CC3)c2CN1]

Tert-butyl 3-((1-((difluoromethoxy)methyl)cyclopropyl)(methyl)carbamoyl)-6-methyl-6,7-dihydropyrazolo[1,5-a]pyrazine-5(4H)-carboxylate (0.108 g, 0.26 mmol) was dissolved in HCl (4 M in dioxane) (1 ml, 4.00 mmol) and the mixture was stirred for 1 h. The reaction mixture was concentrated and used in the next step without further purification.

### Synthesis of indolizine-2-carboxylic Acids

### Synthesis of 1-cyanoindolizine-2-carboxylic Acid

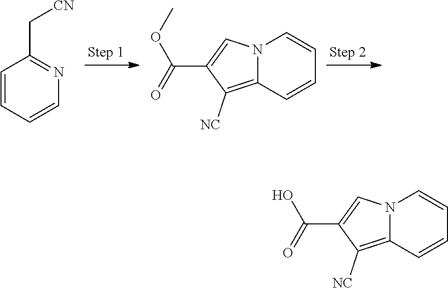

### Step 1

2-(Pyridin-2-yl)acetonitrile (2.42 g, 20.51 mmol) and ethyl 3-bromo-2-oxopropanoate (2.0 g, 10.26 mmol) were mixed in acetone (50 mL) and refluxed for 5 h. The mixture was cooled, the precipitated solid was removed, and the filtrate was concentrated. The residue was triturated with water (50 ml), stirred for 1 h, and the product collected by filtration to give ethyl 1-cyanoindolizine-2-carboxylate (1.9 g, 8.87 mmol, 86.5% yield) as brown solid.

### Step 2

To a suspension of ethyl 1-cyanoindolizine-2-carboxylate (400.44 mg, 1.87 mmol) in THF/H₂O (3 mL/3 mL) was added lithium hydroxide monohydrate (313.77 mg, 7.48 mmol). The mixture was stirred at r.t. for 10 h. The mixture was concentrated; the residue was dissolved in water (10 ml) and acidified with 10% aq. HCl to pH 3. The precipitated solid was collected by filtration and dried to afford 1-cyanoindolizine-2-carboxylic acid (237.0 mg, 1.27 mmol, 68.1% yield) as brown solid.

Rt (Method G) 1.29 mins, m/z 215 [M+H]⁺

¹H NMR (400 MHz, DMSO) δ 6.98 (t, J=6.8 Hz, 1H), 7.25 (dd, J=9.1, 6.7 Hz, 1H), 7.64 (d, J=9.1 Hz, 1H), 8.21 (s, 1H), 8.51 (d, J=7.0 Hz, 1H), 13.10 (br.s, 1H).

### Synthesis of 8-(trifluoromethyl)indolizine-2-carboxylic Acid

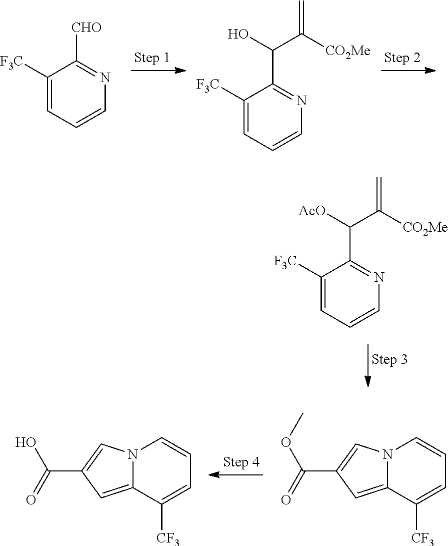 [SMILES: O=Cc1ncccc1C(F)(F)F ; C=C(C(=O)OC)C(O)c1ncccc1C(F)(F)F ; C=C(C(=O)OC)C(OC(C)=O)c1ncccc1C(F)(F)F ; O=C(O)c1cc2c(C(F)(F)F)cccn2c1 ; COC(=O)c1cc2c(C(F)(F)F)cccn2c1]

### Step 1: Methyl 2-{hydroxy [3-(trifluoromethyl)pyridin-2-yl]methyl}prop-2-enoate

To a solution of 3-(trifluoromethyl)pyridine-2-carbaldehyde (5.1 g, 29.12 mmol) and methyl prop-2-enoate (7.52 g, 87.37 mmol, 7.92 mL) in dioxane/H₂O (1/1 v/v, 150 mL), was added 1,4-diazabicyclo[2.2.2]octane (3.27 g, 29.12 mmol). The resulting mixture was stirred at r.t. overnight. The reaction mixture was then diluted with 500 mL of H₂O and extracted with MTBE (300 mL). The organic phase was washed with brine, dried over Na₂SO₄ and concentrated under reduced pressure to give methyl 2-hydroxy[3-(trifluoromethyl)pyridin-2-yl]methylprop-2-enoate (6.1 g, 23.35 mmol, 80.2% yield) as brown oil.

### Step 2: Methyl 2-[(acetyloxyl)[3-(trifluoromethyl)pyridin-2-yl]methyl]prop-2-enoate

Methyl 2-hydroxy[3-(trifluoromethyl)pyridin-2-yl]methylprop-2-enoate (5.9 g, 22.59 mmol) was dissolved in acetic anhydride (57.65 g, 564.75 mmol, 53.38 mL) and heated at 100° C. for 2 h. The reaction mixture was concentrated under reduced pressure, the residue was triturated with MTBE (80 mL) and the solution was quenched with NaHCO₃ sat. aq. 50 mL. The organic phase was separated, washed with brine, dried over Na₂SO₄ and concentrated under reduced pressure to give 6 g of a brown liquid, which was 1/1 mixture of methyl 2-[(acetyloxy)[3-(trifluoromethyl)pyridin-2-yl]methyl]prop-2-enoate (6.0 g, 50.0% purity, 9.89 mmol, 43.8% yield) and cyclized indolizine as shown by ¹H NMR. This mixture was used in the next step without further purification.

### Step 3: Methyl 8-(trifluoromethyl)indolizine-2-carboxylate

The solution of methyl 2-[(acetyloxy)[3-(trifluoromethyl)pyridin-2-yl]methyl]prop-2-enoate (6.0 g, 19.79 mmol) in 100 mL of xylene was heated under reflux overnight. After cooling to r.t. the reaction mixture was diluted with MTBE (200 mL) and washed with Na₂CO₃, brine, dried over Na₂SO₄ and concentrated under reduced pressure to give methyl 8-(trifluoromethyl)indolizine-2-carboxylate (4.67 g, 19.2 mmol, 97% yield) an off-white crystalline solid.

### Step 4: 8-(trifluoromethyl)indolizine-2-carboxylic Acid

To a solution of methyl 8-(trifluoromethyl)indolizine-2-carboxylate (230.0 mg, 945.79 μmol) in methanol (15 mL) was added a solution of sodium hydroxide (113.63 mg, 2.84 mmol) in H₂O (5 mL). The resulting mixture was stirred overnight at r.t. The reaction mixture was concentrated under reduced pressure and the residue was triturated with H₂O (50 mL). The resulting solution was acidified with HCl 5N to pH˜2 and extracted with MTBE (30 mL). The combined organic extract was dried over Na₂SO₄ and concentrated under vacuum to give 8-(trifluoromethyl)indolizine-2-carboxylic acid (180.0 mg, 785.49 μmol, 82.9% yield) as pale brown solid.

Rt (Method G) 1.19 mins, m/z 228 [M−H]⁻, m/z 230 [M+H]⁺

¹H NMR (400 MHz, DMSO-d6) δ 12.61 (s, 1H), 8.55 (d, J=7.1 Hz, 1H), 8.24 (d, J=1.6 Hz, 1H), 7.29 (d, J=6.9 Hz, 1H), 6.79 (t, J=7.9 Hz, 1H), 6.76 (s, 1H).

### Synthesis of 8-fluoroindolizine-2-carboxylic Acid

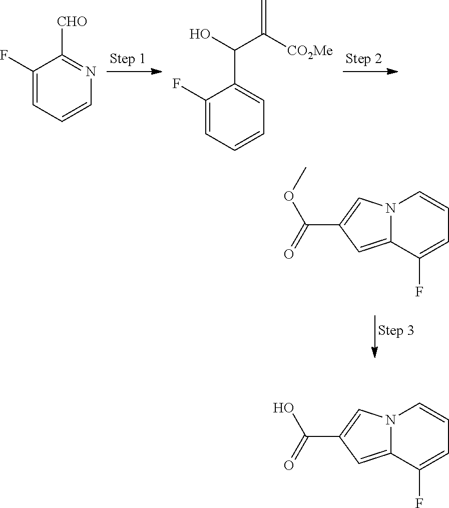

### Step 1: Methyl 2-1(3-fluoropyridin-2-yl)(hydroxy)methyl]prop-2-enoate

To a solution of 3-fluoropyridine-2-carbaldehyde (500.0 mg, 4.0 mmol) in dioxane (10 mL) and H₂O (2 mL), was added methyl prop-2-enoate (412.89 mg, 4.8 mmol, 430.0 μl) and 1,4-diazabicyclo[2.2.2]octane (224.16 mg, 2.0 mmol). The mixture was stirred for 24 hours at r.t. and the volatiles were removed under reduced pressure. The crude residue was partitioned between CHCl₃ (15 mL) and 3% aq. H₃PO₄ (20 mL), and product extracted with CHCl₃ (2*10 mL). The combined organic extracts were dried over Na₂SO₄ and concentrated under reduced pressure to give methyl 2-1(3-fluoropyridin-2-yl)(hydroxy)methyl]prop-2-enoate (430.0 mg, 2.04 mmol, 54.6% yield) as yellow oil.

### Step 2: Methyl 8-fluoroindolizine-2-carboxylate

A mixture of methyl 2-1(3-fluoropyridin-2-yl)(hydroxy)methyl]prop-2-enoate (430.34 mg, 2.04 mmol) and acetic anhydride (5.4 g, 52.9 mmol, 5.0 mL) was heated at 100° C. for 1 h, then 140° C. for 4 h, then cooled and concentrated. The residue was dissolved in EtOAc, and the mixture washed with sat. aq NaHCO₃, dried over Na₂SO₄ and concentrated. The residue was purified by flash column chromatography (hexane-EtOAc 3:2) to afford methyl 8-fluoroindolizine-2-carboxylate (260.0 mg, 1.35 mmol, 50.4% yield) as yellow solid.

### Step 3: 8-fluoroindolizine-2-carboxylic Acid

To a solution of methyl 8-fluoroindolizine-2-carboxylate (259.87 mg, 1.35 mmol) in MeOH-THF (5 ml/5 mL) was added 10% aq. NaOH (107.61 mg, 2.69 mmol,). The mixture was stirred at 65° C. for 5 h. The reaction mixture was concentrated, and the residue dissolved in H₂O (10 mL) and acidified with 10% aq. HCl to pH 4. The precipitate was collected by filtration and dried to afford 8-fluoroindolizine-2-carboxylic acid (200.0 mg, 1.12 mmol, 83% yield) as beige solid.

Rt (Method G) 1.11 mins, m/z 178 [M−H]⁻, m/z 180 [M+H]⁺

¹H NMR (500 MHz, DMSO-d6) δ 12.53 (s, 1H), 8.23-7.99 (m, 2H), 6.79 (s, 1H), 6.72-6.52 (m, 2H).

### Synthesis of 6-fluoroindolizine-2-carboxylic Acid

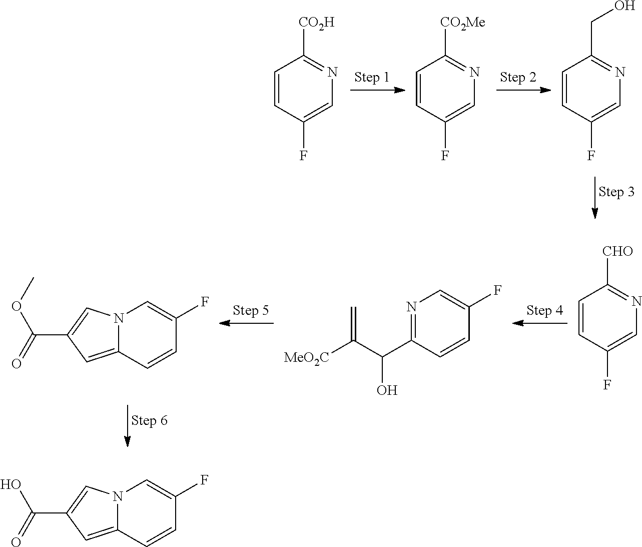

### Step 1: Methyl 5-fluoropyridine-2-carboxylate

To a cooled (0° C.) solution of dry MeOH (25 mL) was added dropwise thionyl chloride (2.53 g, 21.26 mmol). 5-fluoropyridine-2-carboxylic acid (2.0 g, 14.18 mmol) was added and the reaction mixture was heated at 55° C. for 5 h. The reaction mixture was cooled to r.t. and concentrated. The residue was triturated with NaHCO₃ (20 ml sat. aq.) and the H₂O phase was extracted with EtOAc (3*15 mL). The combined organic phases was dried over Na₂SO₄, filtered and concentrated to afford methyl 5-fluoropyridine-2-carboxylate (1.8 g, 11.6 mmol, 81.9% yield) as white solid.

### Step 2: (5-fluoropyridin-2-yl)methanol

To a stirred, cooled (−60° C.) solution of methyl 5-fluoropyridine-2-carboxylate (1.8 g, 11.6 mmol) in dry DCM (50 mL) under Ar was added dropwise diisobutylaluminum hydride (4.13 g, 29.01 mmol, 5.29 mL). The reaction mixture was warmed to r.t. and stirred overnight. The mixture was cooled to −10° C. and HCl 1M was added dropwise. The mixture was stirred for 1 h at r.t. and organic phase was separated. The H₂O phase was extracted with DCM (20 mL). The combined organic phases were dried over Na₂SO₄, filtered and concentrated to afford (5-fluoropyridin-2-yl)methanol (850.0 mg, 6.69 mmol, 57.6% yield) as yellow oil.

### Step 3: 5-fluoropyridine-2-carbaldehyde

To a stirred solution of (5-fluoropyridin-2-yl)methanol (850.0 mg, 6.69 mmol) in dry DCM (15 mL) at r.t. was added portionwise 1,1,1-tris(acetoxy)-1,1-dihydro-1,2-benziodoxol-3 (1H)-one (2.84 g, 6.69 mmol). The mixture was stirred for 2 h and cooled to 0° C., then NaOH 20% aq. (1.2 g, 30.09 mmol) was added dropwise with stirring. The organic phase was separated, dried over Na₂SO₄ and concentrated to afford 5-fluoropyridine-2-carbaldehyde (300.0 mg, 2.4 mmol, 35.9% yield) as yellow solid.

### Step 4: Methyl 2-[5-fluoropyridin-2-yl)(hydroxy)methyl]prop-2-enoate

To a solution of 5-fluoropyridine-2-carbaldehyde (299.08 mg, 2.39 mmol) in dioxane (10 mL) and H₂O (2 mL) was added methyl prop-2-enoate (247.0 mg, 2.87 mmol, 260.0 μl) and 1,4-diazabicyclo[2.2.2]octane (134.09 mg, 1.2 mmol). After 24 hours the volatiles were removed under reduced pressure and the crude residue was partitioned between CHCl₃ (15 mL) and H₂O (25 mL). Product was extracted with CHCl₃ (2*10 mL). The combined organic extracts were dried over Na₂SO₄ and concentrated under reduced pressure to give methyl 2-[(5-fluoropyridin-2-yl)(hydroxy)methyl]prop-2-enoate (410.0 mg, 1.94 mmol, 81.2% yield) as brown oil.

### Step 5: Methyl 6-fluoroindolizine-2-carboxylate

A mixture of methyl 2-[(5-fluoropyridin-2-yl)(hydroxy)methyl]prop-2-enoate (410.0 mg, 1.94 mmol) and acetic anhydride (5.4 g, 52.9 mmol, 5.0 mL) was heated at 100° C. for 2 h (the formation of 0-acetyl intermediate was checked by LCMS). Than the mixture was heated at 140° C. for 15 h, cooled and concentrated in vacuum. The residue was dissolved in EtOAc (30 mL) and washed with NaHCO₃ sat. aq (40 mL) for 1 h at r.t. The organic phase was separated, dried over Na₂SO₄, filtered and concentrated. The residue was purified by column chromatography on silica (hexane-EtOAc 10:1) to afford methyl 6-fluoroindolizine-2-carboxylate (140.0 mg, 724.74 μmol, 37.3% yield) as white solid.

### Step 6: 6-fluoroindolizine-2-carboxylic Acid

To a solution of methyl 6-fluoroindolizine-2-carboxylate (140.18 mg, 725.66 μmol) in MeOH/THF/H₂O (4/4/1) was added 20% aq. sodium hydroxide (58.05 mg, 1.45 mmol). The mixture was heated at 65° C. overnight. The solvent was removed under reduced pressure, and the resulting residue was dissolved in H₂O. The solution was acidified to pH 3-4 with 1M HCl. The precipitated solid was collected by filtration, washed with H₂O and dissolved in EtOAc-THF (2:1). The solution was dried over Na₂SO₄, filtered and concentrated to afford 6-fluoroindolizine-2-carboxylic acid (105.0 mg, 586.11 μmol, 80.8% yield) as beige solid.

Rt (Method G) 1.07 mins, m/z 178 [M−H]⁻, m/z 180 [M+H]⁺

¹H NMR (500 MHz, DMSO-d6) δ 12.57-12.02 (m, 1H), 8.47 (s, 1H), 8.06-7.91 (m, 1H), 7.61-7.44 (m, 1H), 6.91-6.73 (m, 2H).

### Synthesis of 7-fluoroindolizine-2-carboxylic Acid

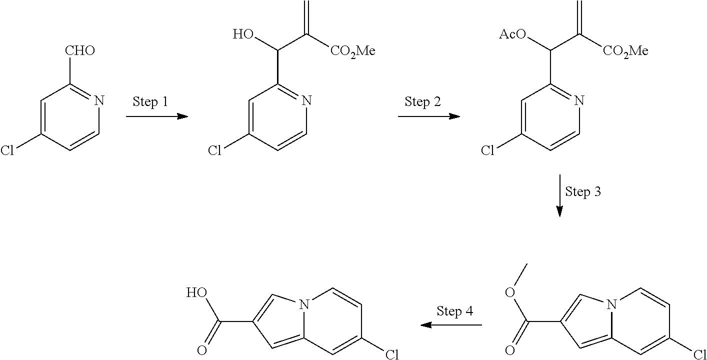 [SMILES: O=Cc1cc(Cl)ccn1 ; C=C(C(=O)OC)C(O)c1cc(Cl)ccn1 ; C=C(C(=O)OC)C(OC(C)=O)c1cc(Cl)ccn1 ; O=C(O)c1cc2cc(Cl)ccn2c1 ; COC(=O)c1cc2cc(Cl)ccn2c1]

### Step 1: Methyl 2[(4-chloropyridin-2-yl)(hydroxy)methyl]prop-2-enoate

To a solution of 4-chloropyridine-2-carbaldehyde (500.0 mg, 3.53 mmol) in dioxane (10 mL) and H₂O was added 20 mL methyl prop-2-enoate (364.9 mg, 4.24 mmol, 380.0 μl) and 1,4-diazabicyclo[2.2.2]octane (198.11 mg, 1.77 mmol). The mixture was stirred at r.t. for 24 hours. The volatiles were removed under reduced pressure and the crude residue was partitioned between CHCl₃ and aqueous diluted phosphoric acid. Product was extracted with CHCl₃ (2*10 mL). The combined organic extracts were dried over Na₂SO₄ and concentrated under reduced pressure to give methyl 2[(4-chloropyridin-2-yl)(hydroxy)methyl]prop-2-enoate (430.0 mg, 1.89 mmol, 53.5% yield) as yellow oil.

### Step 2: Methyl 2-[(acetyloxy)(4-chloropyridin-2-yl)methyl]prop-2-enoate

A mixture of methyl 2[(4-chloropyridin-2-yl)(hydroxy)methyl]prop-2-enoate (430.0 mg, 1.89 mmol) and acetic anhydride (5.4 g, 52.9 mmol, 5.0 mL) was heated at 100° C. for 1 h. The mixture was cooled to r.t., concentrated in vacuo and the residue was partitioned between CHCl₃ (20 mL) and sat. aq NaHCO₃ (30 mL). The organic phase was separated; the H₂O phase was additionally extracted with CHCl₃ (2*5 mL). The combined organic phases were dried over Na₂SO₄, filtered and concentrated to afford methyl 2-[(acetyloxy)(4-chloropyridin-2-yl)methyl]prop-2-enoate (490.0 mg, 1.82 mmol, 96.2% yield) as brown oil, that was used in the next step.

### Step 3: Methyl 7-chloroindolizine-2-carboxylate

A mixture of methyl 2-[(acetyloxy)(4-chloropyridin-2-yl)methyl]prop-2-enoate (490.02 mg, 1.82 mmol) and acetic anhydride (2.16 g, 21.16 mmol, 2.0 mL) was heated at reflux for 3 h. The reaction mixture was cooled, concentrated under vacuum and the residue dissolved in EtOAc (15 mL). The solution was washed with sat. aq. NaHCO₃ then dried over Na₂SO₄ and concentrated. The residue was purified by flash column chromatography (EtOAC-hexane 2:3) to afford methyl 7-chloroindolizine-2-carboxylate (215.0 mg, 1.03 mmol, 56.4% yield) as an orange solid.

### Step 4: 7-chloroindolizine-2-carboxylic Acid

To a solution of methyl 7-chloroindolizine-2-carboxylate (215.0 mg, 1.03 mmol) in MeOH/THF/H₂O (4/4/1) was added 20% aq. NaOH (82.04 mg, 2.05 mmol). The mixture was refluxed at 80° C. overnight. The organic solvent was removed under reduced pressure. The remaining solution was cooled (ice bath, 0-5° C.) and adjusted to pH 3-4 with 1M HCl. The suspension was stirred for 30 minutes, then product was collected by filtration and dried to afforded 7-chloroindolizine-2-carboxylic acid (160.0 mg, 817.99 μmol, 79.8% yield) as yellow solid.

Rt (Method G) 1.17 mins, m/z 194/196 [M−H]⁻, m/z 196/198 [M+H]⁺

¹H NMR (400 MHz, DMSO-d6) δ 12.47 (s, 1H), 8.30 (d, J=7.5 Hz, 1H), 8.04 (s, 1H), 7.59 (s, 1H), 6.75-6.57 (m, 2H).

### Synthesis of 6-fluoroindolizine-2-carboxylic Acid

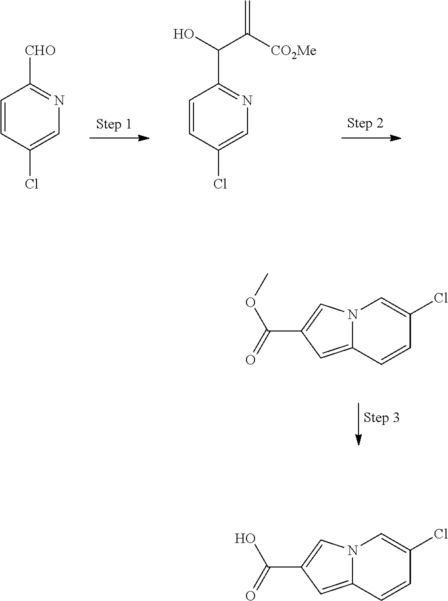

### Step 1: Methyl 2-[(5-chloropyridin-2-yl)(hydroxy)methyl]prop-2-enoate

To a solution of 5-chloropyridine-2-carboxaldehyde (1.0 g, 7.08 mmol) in 20 ml of dioxane and H₂O (4 mL) was added methyl prop-2-enoate (731.5 mg, 8.5 mmol, 770.0 μl) and 1,4-diazabicyclo[2.2.2]octane (397.16 mg, 3.54 mmol). After 24 hours the volatiles were removed under reduced pressure and the crude mixture was partitioned between CHCl₃ and H₂O. Product was extracted with CHCl₃ (2*10 mL). The combined organic extracts were dried over Na₂SO₄ and concentrated under reduced pressure to give methyl 2-[(5-chloropyridin-2-yl)(hydroxy)methyl]prop-2-enoate (1.48 g, 6.5 mmol, 91.8% yield) as yellow oil.

### Step 2: Methyl 6-chloroindolizine-2-carboxylate

A mixture of methyl 2-[(5-chloropyridin-2-yl)(hydroxy)methyl]prop-2-enoate (1.48 g, 6.5 mmol) and acetic anhydride (16.2 g, 158.69 mmol, 15.0 mL) was heated at 100° C. for 2 h (the formation of O-acetyl intermediate was checked by LCMS). The mixture was heated at 140° C. for 10 h, then cooled and concentrated in vacuum. The residue was dissolved in EtOAc, sat. aq. NaHCO₃ was added and the mixture was stirred for 1 h at r.t. The organic phase was separated, dried over Na₂SO₄, filtered and concentrated. The residue was purified with column chromatography on silica (hexane-EtOAc from 10:1 to 4:1 gradient) to afford methyl 6-chloroindolizine-2-carboxylate (400.0 mg, 1.91 mmol, 29.3% yield) as white solid.

### Step 3: 6-chloroindolizine-2-carboxylic Acid

To a solution of methyl 6-chloroindolizine-2-carboxylate (399.75 mg, 1.91 mmol) in MeOH/THF/H₂O (4/4/1) was added 20% aqueous sodium hydroxide (152.54 mg, 3.81 mmol). The mixture was heated at 65° C. overnight. The solvent was removed under reduced pressure. The residue was dissolved in H₂O and the solution adjusted to pH 3-4 with 1M HCl. The suspension was stirred for 30 minutes, and product was collected by filtration and dried over Na₂SO₄ to afford 6-chloroindolizine-2-carboxylic acid (305.0 mg, 1.56 mmol, 81.8% yield) as beige solid.

Rt (Method G) 1.05 mins, m/z 194/196 [M−H]⁻, m/z 196/198 [M+H]⁺

¹H NMR (400 MHz, DMSO-d6) δ 12.46 (s, 1H), 8.55 (s, 1H), 8.01 (s, 1H), 7.51 (d, J=9.6 Hz, 1H), 6.86-6.70 (m, 2H).

### Synthesis of 7-chloro-6-fluoroindolizine-2-carboxylic Acid

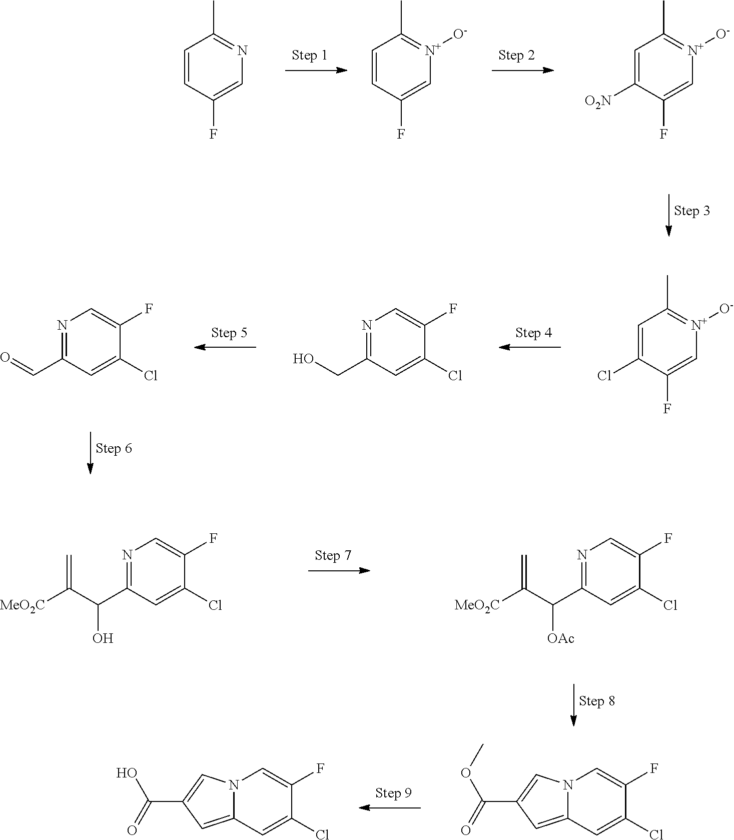

### Step 1: 5-fluoro-2-methylpyridin-1-ium-1-olate

To a cooled (5° C.) solution of 5-fluoro-2-methylpyridine (15.0 g, 134.99 mmol) (1.0 eq) in CH₂Cl₂ (300 mL) was added portionwise 3-chlorobenzene-1-carboperoxoic acid (34.94 g, 202.49 mmol) (1.5 eq). The resulting solution was stirred at room temperature overnight. After stirring for 16 hours the solution was washed with aqueous sodium bicarbonate and the aqueous re-extracted with dichloromethane (3×200 mL). The combined organic fractions were dried and concentrated to give crude 5-fluoro-2-methylpyridin-1-ium-1-olate (11.0 g, 93.0% purity, 80.48 mmol, 59.6% yield).

### Step 2: 5-fluoro-2-methyl-4-nitropyridin-1-ium-1-olate

A mixture of H₂SO₄ (50 mL conc.) and fuming nitric acid (81 mL) was added dropwise over 10 min with ice-cooling (5° C.) and stirring to a solution of 5-fluoro-2-methylpyridin-1-ium-1-olate (11.0 g, 86.53 mmol) in concentrated sulfuric acid (40 mL). The mixture was allowed to warm to room temperature over 1 h and then heated on a steam bath for 2 h. After cooling, the solution was poured onto ice and neutralized by addition of solid ammonium carbonate. The mixture was extracted with CHCl₃ (3×35 mL), dried (Na₂SO₄), and concentrated in vacuo to a solid which was triturated with petroleum ether (60/80), to give 5-fluoro-2-methyl-4-nitropyridin-1-ium-1-olate (8.37 g, 90.0% purity, 43.77 mmol, 50.6% yield) as yellow solid.

### Step 3: 4-chloro-5-fluoro-2-methylpyridin-1-ium-1-olate

Phosphoryl trichloride (22.37 g, 145.91 mmol, 13.6 mL) (3 eq.) in dichloromethane (170 mL) was added dropwise under Ar to a cooled (5-10° C.), stirred solution of 5-fluoro-2-methyl-4-nitropyridine-1-oxide (8.37 g, 48.64 mmol) (1 eq) in dichloromethane (170 mL). After standing for 16 h at room temperature, the solution was refluxed for 4 h, cooled, and poured onto ice (350 g). The mixture was stirred for 10 min and then adjusted to pH13 with cooling, using 40% sodium hydroxide. The aqueous phase was separated and then extracted with dichloromethane. The combined extracts were dried (Na₂SO₄) and concentrated in vacuo. The resulting solid was triturated with petroleum ether (60/80), collected by filtration and dried, to give 4-chloro-5-fluoro-2-methylpyridin-1-ium-1-olate (6.72 g, 97.0% purity, 40.35 mmol, 83% yield).

### Step 4: (4-chloro-5-fluoropyridin-2-yl)methanol

Trifluoroacetyl 2,2,2-trifluoroacetate (1.76 g, 8.36 mmol, 1.17 mL) (3 eq.) was added dropwise over 1 min to a stirred, cooled (10-15° C.) solution of 4-chloro-5-fluoro-2-methylpyridin-1-ium-1-olate (450.0 mg, 2.79 mmol) (1 eq.) in dichloromethane (10 mL). The solution was warmed to room temperature and left for 7 days. It was poured onto ice, the pH was adjusted to 13 by addition K₂CO₃ aq sat. and 40% aq. NaOH. The aqueous layer was separated and further extracted with dichloromethane (15 mL), and the combined organic layers were dried over K₂CO₃ and concentrated to give crude product as red oil. Pure (4-chloro-5-fluoropyridin-2-yl)methanol (92.0 mg, 97.0% purity, 552.36 μmol, 19.8% yield) was obtained after purification by HPLC.

### Step 5: 4-chloro-5-fluoropyridine-2-carbaldehyde

To a cooled (0° C.) solution of (4-chloro-5-fluoropyridin-2-yl)methanol (500.0 mg, 3.09 mmol) in DCM (30 mL) was added 1,1,1-tris(acetoxy)-1,1-dihydro-1,2-benziodoxol-3 (1H)-one (1.44 g, 3.41 mmol) in one portion. After reaction was complete (monitored by ¹H NMR,) the mixture was poured into a stirred aqueous solution of NaHCO₃ and Na₂S₂O₃ and stirred until the organic phase became transparent (about 1 h.). The layers were separated and the aqueous layer extracted with DCM (3×50 mL). The combined organic extracts were washed with brine, dried over Na₂SO₄ and concentrated under reduced pressure to give 4-chloro-5-fluoropyridine-2-carbaldehyde (400.0 mg, 90.0% purity, 2.26 mmol, 72.9% yield).

### Step 6: Methyl 2-[(4-chloro-5-fluoropyridin-2-yl)(hydroxy)methyl]prop-2-enoate

To a solution of 4-chloro-5-fluoropyridine-2-carbaldehyde (1.21 g, 7.6 mmol) in 18 ml of dioxane and H₂O (6 mL) was added methyl prop-2-enoate (850.0 mg, 9.87 mmol, 890.0 μl) and 1,4-diazabicyclo[2.2.2]octane (76.69 mg, 683.7 μmol). After 24 hours the volatiles were removed under reduced pressure and the residue was partitioned between CHCl₃ (100 mL) and H₂O (30 mL). The H₂O layer was extracted with CHCl₃ (2*30 mL). The combined organic extracts were dried over Na₂SO₄ and concentrated under reduced pressure to give methyl 2-[(4-chloro-5-fluoropyridin-2-yl)(hydroxy)methyl]prop-2-enoate (1.5 g, 95.0% purity, 5.8 mmol, 76.4% yield) as yellow oil.

### Step 7: Methyl 2-[(acetyloxy)(4-chloro-5-fluoropyridin-2-yl)methyl]prop-2-enoate

A single-neck round bottomed flask equipped with a magnetic stirrer and a reflux condenser was charged with acetic anhydride (43.65 g, 427.54 mmol) and methyl 2-[(4-chloro-5-fluoropyridin-2-yl)(hydroxy)methyl]prop-2-enoate (1.5 g, 6.11 mmol). The reaction mixture was stirred at 100° C. for 3 hours to give methyl 2-[(acetyloxy)(4-chloro-5-fluoropyridin-2-yl)methyl]prop-2-enoate (1.5 g, 95.0% purity, 4.95 mmol, 81.1% yield) as solution in Ac₂O.

### Step 8: Methyl 7-chloro-6-fluoroindolizine-2-carboxylate

The solution of methyl 2-[(acetyloxy)(4-chloro-5-fluoropyridin-2-yl)methyl]prop-2-enoate (1.5 g, 5.21 mmol) in Ac₂O was heated under reflux under N₂ for 96 hours. The reaction mixture was cooled to room temperature, then poured into a mixture of ice and sat. aq NaHCO₃, and stirred for 1 hour. The mixture was extracted with ethyl acetate (3×25 mL). The combined organic extracts were dried over anhydrous Na₂SO₄, filtered and concentrated. The crude product was purified by silica gel column chromatography eluting with hexane/ethyl acetate (3/1) to afford methyl 7-chloro-6-fluoroindolizine-2-carboxylate (770.0 mg, 98.0% purity, 3.32 mmol, 63.6% yield) as white solid.

### Step 9: 7-chloro-6-fluoroindolizine-2-carboxylic Acid

To a solution of methyl 7-chloro-6-fluoroindolizine-2-carboxylate (600.0 mg, 2.64 mmol) in MeOH/THF/H₂O (4/4/1) (20 mL) was added sodium hydroxide (527.13 mg, 13.18 mmol). The mixture was refluxed at 80° C. for 6 h. Volatiles were removed under reduced pressure. The remaining solution was cooled (0-5° C.) and adjusted to pH 3-4 with 1M HCl. The suspension was stirred for 30 minutes and product was collected by filtration. The filter cake was dried to give 7-chloro-6-fluoroindolizine-2-carboxylic acid (440.0 mg, 98.0% purity, 2.02 mmol, 76.6% yield) as yellow solid.

Rt (Method G) 1.24 mins, m/z 212/214 [M−H]⁻, m/z 214/216 [M+H]⁺

¹H NMR (400 MHz, DMSO-d6) δ 12.56 (s, 1H), 8.69 (d, J=5.6 Hz, 1H), 8.03 (s, 1H), 7.84 (d, J=7.6 Hz, 1H), 6.79 (s, 1H).

### Synthesis of 6,8-dichloroindolizine-2-carboxylic Acid

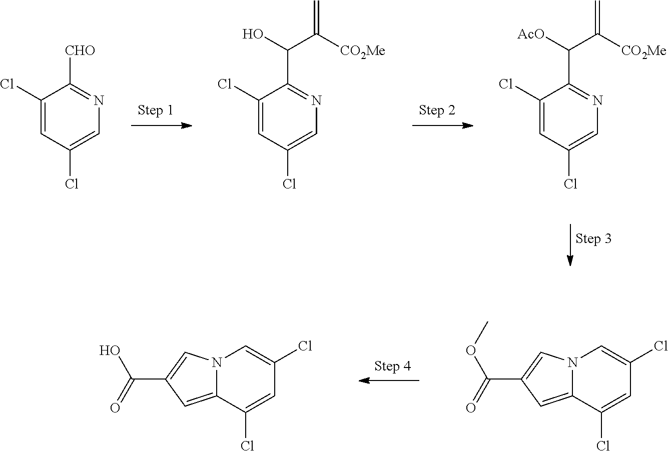 [SMILES: O=Cc1ncc(Cl)cc1Cl ; C=C(C(=O)OC)C(O)c1ncc(Cl)cc1Cl ; C=C(C(=O)OC)C(OC(C)=O)c1ncc(Cl)cc1Cl ; O=C(O)c1cc2c(Cl)cc(Cl)cn2c1 ; COC(=O)c1cc2c(Cl)cc(Cl)cn2c1]

### Step 1: Methyl 2-[(3,5-dichloropyridin-2-yl)(hydroxy)methyl]prop-2-enoate

To a solution of 3,5-dichloropyridine-2-carbaldehyde (462.0 mg, 2.62 mmol) and methyl prop-2-enoate (677.97 mg, 7.88 mmol, 710.0 μl) in dioxane/H₂O (1/1 v/v) (15 mL) was added 1,4-diazabicyclo[2.2.2]octane (294.46 mg, 2.63 mmol). The resulting mixture was stirred at r.t. overnight. The reaction mixture was diluted with H₂O (200 mL) and extracted with 50 mL of MTBE. The organic phase was washed with brine, dried over Na₂SO₄ and concentrated under reduced pressure to give methyl 2[(3,5-dichloropyridin-2-yl)(hydroxy)methyl]prop-2-enoate (570.0 mg, 2.17 mmol, 82.8% yield) as brown oil.

### Step 2: Methyl 2-[(acetyloxy)(3,5-dichloropyridin-2-yl)methyl]prop-2-enoate

Methyl 2-[(3,5-dichloropyridin-2-yl)(hydroxy)methyl]prop-2-enoate (570.0 mg, 2.17 mmol) was dissolved in acetic anhydride (5.55 g, 54.34 mmol, 5.14 mL) and heated at 100° C. for 2 h. The reaction mixture was concentrated under reduced pressure, the residue was taken up in 20 mL of MTBE and the resulting mixture was quenched with sat. aq NaHCO₃. The organic phase was separated, washed with brine, dried over Na₂SO₄ and concentrated under reduced pressure to give methyl 2-[(acetyloxy)(3,5-dichloropyridin-2-yl)methyl]prop-2-enoate (450.0 mg, 1.48 mmol, 68.1% yield) as brown liquid.

### Step 3: Methyl 6,8-dichloroindolizine-2-carboxylate

Methyl 2-[(acetyloxy)(3,5-dichloropyridin-2-yl)methyl]prop-2-enoate (440.0 mg, 1.45 mmol) was dissolved in 15 mL of xylene and refluxed overnight. The reaction mixture was cooled to r.t., diluted with MTBE (50 mL), quenched with NaHCO₃ aq (30 mL), washed with brine, dried over Na₂SO₄ and concentrated under reduced pressure to give methyl 6,8-dichloroindolizine-2-carboxylate (360.0 mg, 1.47 mmol, 98.9% yield) as light yellow crystals.

### Step 4: 6,8-dichloroindolizine-2-carboxylic Acid

To a solution of methyl 6,8-dichloroindolizine-2-carboxylate (360.0 mg, 1.47 mmol) in methanol 50 (mL) was added a solution of sodium hydroxide (589.92 mg, 14.75 mmol) in H₂O (10 mL). The resulting mixture was stirred overnight at r.t. The reaction mixture was concentrated under reduced pressure and the residue was taken up in H₂O (100 mL). The resulting solution was acidified with 5N HCl to pH˜2 and extracted with MTBE (2×100 mL). The combined organic extracts were dried over Na₂SO₄ and concentrated in vacuum to give 6,8-dichloroindolizine-2-carboxylic acid (220.0 mg, 956.32 μmol, 64.8% yield) as yellow powder.

Rt (Method G) 1.29 mins, m/z 228/230 [M−H]⁻, m/z 230/232 [M+H]⁺

¹H NMR (400 MHz, DMSO-d6) δ 12.62 (s, 1H), 8.62 (s, 1H), 8.14 (d, J=1.7 Hz, 1H), 7.15 (d, J=1.5 Hz, 1H), 6.85 (s, 1H).

### Synthesis of 5-methylindolizine-2-carboxylic Acid

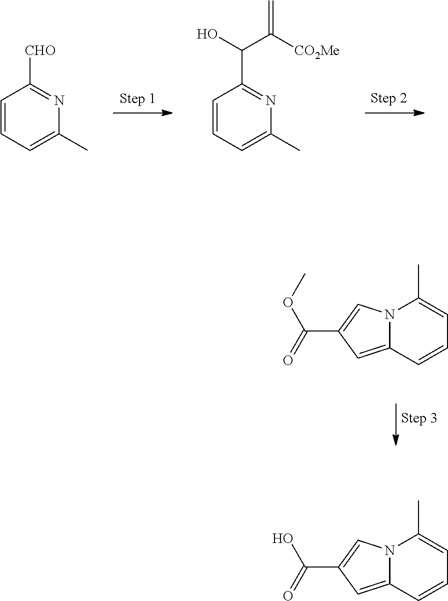

### Step 1: Methyl 2-[hydroxy(6-methylpyridin-2-yl)methyl]prop-2-enoate

To a solution of 6-methylpyridine-2-carbaldehyde (500.0 mg, 4.13 mmol) in dioxane (10 mL) and H₂O (2 mL) was added methyl prop-2-enoate (426.24 mg, 4.95 mmol, 450.0 μl) and 1,4-diazabicyclo[2.2.2]octane (231.41 mg, 2.06 mmol). The mixture was stirred for 24 hours, the volatiles were removed under reduced pressure and the crude mixture was partitioned between CHCl₃ and H₂O. Product was extracted with CHCl₃ (2*10 mL). The combined organic extracts were dried over Na₂SO₄ and concentrated under reduced pressure to give methyl 2-[hydroxy(6-methylpyridin-2-yl)methyl]prop-2-enoate (580.0 mg, 2.8 mmol, 67.8% yield) as white solid.

### Step 2: Methyl 5-methylindolizine-2-carboxylate

A mixture of methyl 2-[hydroxy(6-methylpyridin-2-yl)methyl]prop-2-enoate (580.46 mg, 2.8 mmol) and acetic anhydride (5.4 g, 52.9 mmol, 5.0 mL) was heated at 100° C. for 3 h. The mixture was cooled and concentrated under reduced pressure. The residue was dissolved in EtOAc (20 mL) and washed with sat. aq NaHCO₃ The organic phase was dried over Na₂SO₄, filtered and concentrated. The residue was purified by flash column chromatography (hexane-EtOAc 3:1) to afford methyl 5-methylindolizine-2-carboxylate (350.0 mg, 1.85 mmol, 66% yield) as beige solid.

### Step 3: 5-methylindolizine-2-carboxylic Acid

To a solution of methyl 5-methylindolizine-2-carboxylate (350.05 mg, 1.85 mmol) in MeOH (5 mL) was added 20% H₂O solution of sodium hydroxide (147.99 mg, 3.7 mmol). The reaction mixture was heated at 65° C. for 3 h. The mixture was cooled, and concentrated; the residue was dissolved in H₂O and acidified with 2M HCl 2M to pH 4. The precipitated solid was collected by filtration, and dried to afford 5-methylindolizine-2-carboxylic acid (250.0 mg, 1.43 mmol, 77.1% yield) as grey solid.

Rt (Method G) 1.16 mins, m/z 176 [M+H]⁺

¹H NMR (500 MHz, DMSO-d6) δ 12.35 (s, 1H), 7.79 (s, 1H), 7.40 (d, J=9.1 Hz, 1H), 6.80 (s, 1H), 6.76 (dd, J=9.3, 6.8 Hz, 1H), 6.56 (d, J=6.6 Hz, 1H), 2.52 (s, 3H).

### Synthesis of 3-propylindolizine-2-carboxylic Acid

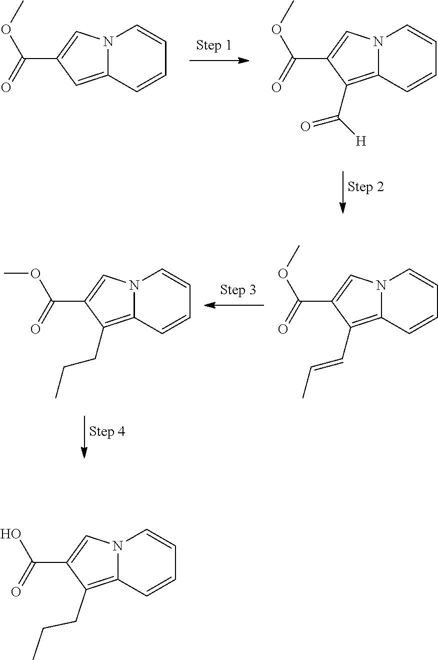 [SMILES: COC(=O)c1cc2ccccn2c1 ; COC(=O)c1cn2ccccc2c1C=O ; CCCc1c(C(=O)OC)cn2ccccc12 ; C/C=C/c1c(C(=O)OC)cn2ccccc12 ; CCCc1c(C(=O)O)cn2ccccc12]

### Step 1: Methyl 3-formylindolizine-2-carboxylate

A solution of phosphoryl trichloride (14.89 g, 97.09 mmol, 9.05 mL) (1.7 eq) in DMF (300 mL) was stirred at 0° C. for 1 h. To a cooled (0° C.) stirred solution of methyl indolizine-2-carboxylate (10.01 g, 57.11 mmol) in dry CH₂Cl₂ (1100 mL), was added ⅔ of the previously prepared POCl₃ solution in DMF (200 ml, 1.1 eq.). After being stirred at r.t. for 2 h, the reaction mixture was quenched with aqueous sat. NaHCO₃. The organic layer was washed with H₂O (0.5 L), dried over Na₂SO₄ and concentrated under reduced pressure to give methyl 3-formylindolizine-2-carboxylate (8.0 g, 95.0% purity, 37.4 mmol, 65.5% yield) which was used in the next step without further purification.

### Step 2: Methyl 3-[(1E)-prop-1-en-1-yl]indolizine-2-carboxylate

To a cooled (−15° C.) solution of ethyl(trisphenyl)phosphonium bromide (13.7 g, 36.91 mmol) in anhydrous THF (200 mL) under Ar was slowly added n-BuLi (16 mL, 2.5 M in n-hexane). The mixture was warmed to r.t. and stirred for 1.5 h. Then a solution of methyl 3-formylindolizine-2-carboxylate (2.5 g, 12.3 mmol) in anhydrous THF (50 mL) was added dropwise to the solution, and the reaction stirred at r.t. for another 24 h. The reaction mixture was cooled and quenched by addition of H₂O (200 mL). MTBE (150 mL) was added and the resulting mixture was stirred at r.t. for 15 min. The organic layer was separated, dried over Na₂SO₄, filtered and the filtrate concentrated under reduced pressure. The crude product was purified by HPLC to give methyl 3-[(1E)-prop-1-en-1-yl]indolizine-2-carboxylate (500.0 mg, 95.0% purity, 2.21 mmol, 17.9% yield).

### Step 3: Methyl 3-propylindolizine-2-carboxylate

To a solution of methyl 3-[(1E)-prop-1-en-1-yl]indolizine-2-carboxylate (150.0 mg, 696.85 μmol) in THF (5 mL) was added 10% Pd on carbon (5% mass). The mixture was hydrogenated at 1 bar and then allowed to stir at r.t for 1 h. NMR monitoring). The reaction mixture was filtered. The filtrate was concentrated under reduced pressure to give crude methyl 3-propylindolizine-2-carboxylate (150.0 mg, 91.0% purity, 628.27 μmol, 90.2% yield), which was used in the next step without further purification

### Step 4: 3-propylindolizine-2-carboxylic Acid

Methyl 3-propylindolizine-2-carboxylate (399.81 mg, 1.84 mmol) and lithium hydroxide monohydrate (108.11 mg, 2.58 mmol) were stirred in a mixture of THF:H₂O:CH₃OH (v/v 3:1:1, 50 mL) at 50° C. for 18 h. The reaction mixture was then concentrated under reduced pressure and acidified to pH 4 with saturated solution of citric acid. The product was collected by filtration, washed with H₂O (3×50 mL), and then dried in vacuo at 45° C. to give 3-propylindolizine-2-carboxylic acid (244.0 mg, 94.0% purity, 1.13 mmol, 61.3% yield) as a yellow solid.

Rt (Method G) 1.32 mins, m/z 204 [M+H]⁺

¹H NMR (500 MHz, DMSO-d6) δ 12.17 (s, 1H), 8.11 (d, J=7.1 Hz, 1H), 7.41 (d, J=9.0 Hz, 1H), 6.75-6.67 (m, 2H), 6.63 (t, J=6.4 Hz, 1H), 3.22 (t, J=7.7 Hz, 2H), 1.56 (h, J=7.4 Hz, 2H), 0.89 (t, J=7.4 Hz, 3H).

### Synthesis of 5-chloroindolizine-2-carboxylic Acid

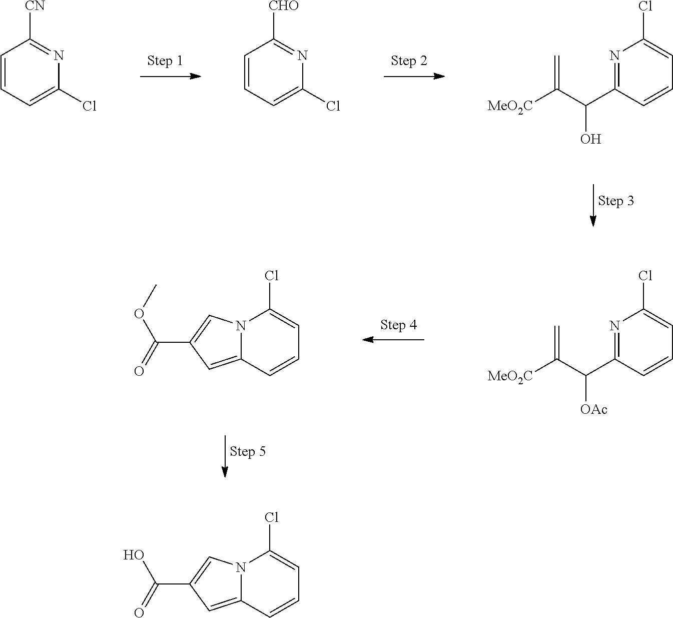 [SMILES: N#Cc1cccc(Cl)n1 ; O=Cc1cccc(Cl)n1 ; C=C(C(=O)OC)C(O)c1cccc(Cl)n1 ; COC(=O)c1cc2cccc(Cl)n2c1 ; C=C(C(=O)OC)C(OC(C)=O)c1cccc(Cl)n1 ; O=C(O)c1cc2cccc(Cl)n2c1]

### Step 1: 6-chloropicolinaldehyde

To a cooled (−78° C.), stirred solution of 6-chloropicolinonitrile (15.0 g, 108 mmol) in dichloromethane (500 mL) under flow of argon was added DIBAL-H (23 mL). The reaction mixture was stirred for 3 h maintaining the temperature below −60° C. Then, the mixture was cooled to −78° C. and the reaction was quenched with H₂O (46 mL). The suspension obtained was warmed to r.t. and acidified to pH 4 with hydrochloric acid (approx. 50 mL). The organic layer was separated, washed with brine, dried over Na₂SO₄, and concentrated under reduced pressure. The residue was purified by silica gel column chromatography (hexane/ethyl acetate 8:2) to give 4.15 g (29.3 mmol, 27%) of 6-chloropicolinaldehyde.

### Step 2: methyl 2-((6-chloropyridin-2-yl)(hydroxy)methyl)acrylate

To a mixture of 6-chloropicolinaldehyde (3.15 g, 22.3 mmol), dioxane (27 mL), and H₂O (9 mL) was added methyl methacrylate (2.30 g, 23.0 mmol) and DABCO (0.250 g, 2.23 mmol). The mixture was stirred at r.t. overnight. The mixture was diluted with H₂O (50 mL) and extracted with ethyl acetate (2×50 mL). The combined organic extracts were washed with H₂O and brine, then dried over Na₂SO₄, and evaporated under reduced pressure to obtain 5.00 g (22.0 mmol, 99%) of methyl 2-((6-chloropyridin-2-yl)(hydroxy)methyl)acrylate.

### Step 3: methyl 2-[(acetyloxy)(6-chloropyridin-2-yl)methyl]prop-2-enoate

A mixture of methyl 2-((6-chloropyridin-2-yl)(hydroxy)methyl)acrylate (5.00 g, 22.0 mmol) and acetic anhydride (40 mL) was stirred at 100° C. overnight, cooled to r.t., and used in the next step without further purification.

### Step 4: methyl 5-chloroindolizine-2-carboxylate

A solution of methyl 2-(acetoxy(6-chloropyridin-2-yl)methyl)acrylate, obtained in the previous step, was poured into H₂O (250 mL) and extracted with MTBE (2×70 mL). The organic extract was washed with H₂O (3×100 mL) and NaHCO₃ solution (3×100 mL), dried over Na₂SO₄, and concentrated under reduced pressure. The obtained solid was purified by silica gel column chromatography (hexane/ethyl acetate 8:2) to give 2.00 g (9.54 mmol, 44%) of compound methyl 5-chloroindolizine-2-carboxylate.

### Step 5: 5-chloroindolizine-2-carboxylic Acid

To a mixture of methyl 5-chloroindolizine-2-carboxylate (1.20 g, 5.72 mmol), THF (8 mL), methanol (8 mL), and H₂O (4 mL) was added a solution of NaOH (0.275 g, 6.88 mmol) in H₂O (3 mL). The mixture was stirred at r.t. for 1 h. Volatiles were evaporated and the residue was mixed with H₂O (10 mL). The obtained solution was acidified with NaHSO₄ (3.00 g). The precipitated solid was collected by filtration, washed with H₂O, and dried to obtain 1.10 g (5.62 mmol, 98%) of 5-chloroindolizine-2-carboxylic acid.

Rt (Method G) 1.18 mins, m/z 196 [M+H]⁺

¹H NMR (400 MHz, DMSO-d6) δ 12.63 (s, 1H), 7.97 (s, 1H), 7.57 (d, J=9.0 Hz, 1H), 7.04-6.90 (m, 2H), 6.84 (t, J=8.0 Hz, 1H).

### Synthesis of 8-chloroindolizine-2-carboxylic Acid

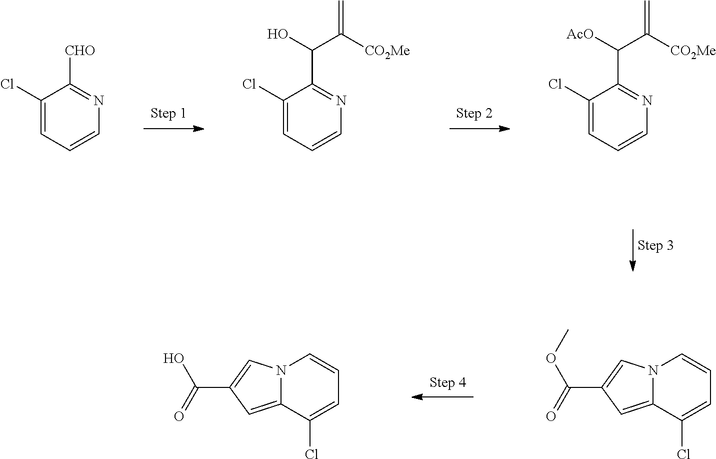 [SMILES: O=Cc1ncccc1Cl ; C=C(C(=O)OC)C(O)c1ncccc1Cl ; C=C(C(=O)OC)C(OC(C)=O)c1ncccc1Cl ; O=C(O)c1cc2c(Cl)cccn2c1 ; COC(=O)c1cc2c(Cl)cccn2c1]

### Step 1: methyl 2-((3-chloropyridin-2-yl)(hydroxy)methyl)acrylate

To a mixture of 3-chloropicolinaldehyde (7.10 g, 50.2 mmol), dioxane (60 mL), and H₂O (20 mL) were added methyl acrylate (5.40 mL, 59.6 mmol) and DABCO (0.340 g, 3.03 mmol. The reaction mixture was stirred at r.t. overnight. The mixture was diluted with ethyl acetate (100 mL) and H₂O (50 mL). The aqueous layer was separated and extracted with ethyl acetate (2×50 mL). The combined organic extracts were washed with H₂O and brine, dried over Na₂SO₄, and concentrated under reduced pressure to obtain 8.00 g (35.1 mmol, 70%) of methyl 24(3-chloropyridin-2-yl)(hydroxy)methyl)acrylate.

### Step 2: 2-[(acetyloxy)(3-chloropyridin-2-yl)methyl]prop-2-enoic Acid

A mixture of methyl 2-((3-chloropyridin-2-yl)(hydroxy)methyl)acrylate (8.00 g, 35.1 mmol) and acetic anhydride (100 mL) was stirred at 100° C. for 3 h, then concentrated under reduced pressure (80° C.). The residue was used in the next step without further purification.

### Step 3: methyl 8-chloroindolizine-2-carboxylate

Methyl 2-(acetoxy(3-chloropyridin-2-yl)methyl)acrylate was mixed with H₂O and extracted with MTBE. The organic extract was washed with sat. aq. NaHCO₃, dried over Na₂SO₄, and concentrated under reduced pressure to give 5.90 g (28.1 mmol, 80% over 2 steps) of methyl 8-chloroindolizine-2-carboxylate.

### Step 4: 8-chloroindolizine-2-carboxylic Acid

To a mixture of methyl 8-chloroindolizine-2-carboxylate (2.50 g, 11.9 mmol), THF (8 mL), methanol (8 mL), and H₂O (2 mL) was added a solution of NaOH (1.43 g, 35.7 mmol) in H₂O (7 mL). The mixture was stirred at r.t. overnight, then the volatiles were evaporated and the residue was mixed with H₂O. The obtained slurry was washed with ethyl acetate and then acidified with hydrochloric acid. The precipitate was collected by filtration, then dried to obtain 2.00 g (10.2 mmol, 86%) of 8-chloroindolizine-2-carboxylic acid.

Rt (Method G) 1.08 mins, m/z 194 [M−H]⁻

¹H NMR (400 MHz, DMSO-d6) δ 12.55 (s, 1H), 8.31 (d, J=7.0 Hz, 1H), 8.17 (d, J=1.8 Hz, 1H), 6.97 (d, J=7.1 Hz, 1H), 6.78 (s, 1H), 6.67 (t, J=7.1 Hz, 1H).

### Synthesis of 5-(trifluoromethyl)indolizine-2-carboxylic Acid

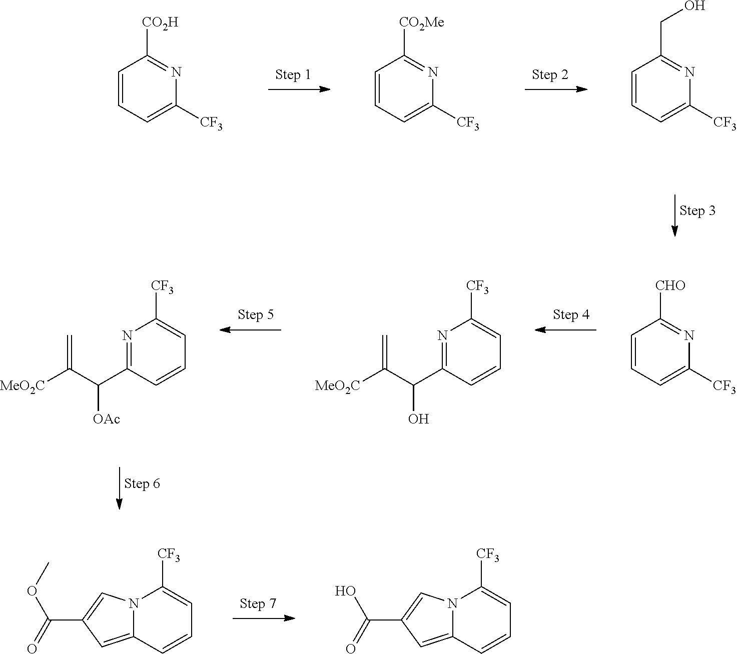

### Step 1: Methyl 6-(trifluoromethyl)pyridine-2-carboxylate

To a stirred solution of 6-(trifluoromethyl)pyridine-2-carboxylic acid (8.8 g, 46.05 mmol) in dry MeOH (150 mL) was added carefully sulfuric acid (6.77 g, 69.07 mmol, 3.76 mL). The resulting mixture was refluxed overnight. The reaction mixture was concentrated under reduced pressure and the residue partitioned between MTBE (200 mL) and sat. aq NaHCO₃ (200 mL). The organic phase was washed with brine, dried over Na₂SO₄ and concentrated under reduced pressure to give methyl 6-(trifluoromethyl)pyridine-2-carboxylate (8.0 g, 39.0 mmol, 84.7% yield) as light yellow crystalline solid.

### Step 2: [6-(trifluoromethyl)pyridin-2-yl]methanol

To a stirred solution of methyl 6-(trifluoromethyl)picolinate (8.0 g, 39.0 mmol) in dry toluene (200 mL) at r.t. was added dropwise diisobutylaluminum hydride (16.64 g, 117.0 mmol, 112.5 mL) was added dropwise. The resulting mixture was stirred at r.t. overnight. The reaction mixture was quenched with HCl (1M, 50 mL) solution, then basified with 10% aq. NaOH aq until the precipitate dissolved. The organic phase was washed with brine, dried over Na₂SO₄ and concentrated over reduced pressure to give [6-(trifluoromethyl)pyridin-2-yl]methanol (6.0 g, 96.0% purity, 32.52 mmol, 83.4% yield) as yellow liquid.

### Step 3: 6-(trifluoromethyl)pyridine-2-carbaldehyde

To a solution of (6-(trifluoromethyl)pyridin-2-yl)methanol (6.0 g, 33.87 mmol) in 100 mL of DCM was added 1,1,1-tris(acetoxy)-1,1-dihydro-1,2-benziodoxol-3(1H)-one (17.24 g, 40.65 mmol) in a few portions, maintaining temperature below 30° C. (H₂O cooling bath). After reaction was complete (monitored by ¹H NMR), the mixture was poured into a stirred aqueous solution of Na₂CO₃ and Na₂S₂O₃ and stirred until the organic phase became transparent (about 15 min). The layers were separated and the aqueous layer was extracted with DCM (50 mL). The combined organic extracts were washed with brine, dried over Na₂SO₄ and concentrated under reduced pressure to give 6-(trifluoromethyl)pyridine-2-carbaldehyde (9.0 g, 33.0% purity, 16.96 mmol, 50.1% yield) as light brown liquid.

### Step 4: Methyl 2-{hydroxy[6-(trifluoromethyl)pyridin-2-yl]methyl}prop-2-enoate

To a solution of 6-(trifluoromethyl)pyridine-2-carbaldehyde (3.3 g, 18.85 mmol) and methyl prop-2-enoate (4.87 g, 56.53 mmol, 5.12 mL) in dioxane/H₂O (1/1 v/v) (100 mL) was added 1,4-diazabicyclo[2.2.2]octane (2.11 g, 18.84 mmol). The mixture was stirred at r.t. overnight. The mixture was then diluted with H₂O (300 mL) and extracted with MTBE (3×100 mL). The combined organic extracts were washed with brine, dried over Na₂SO₄ and concentrated under reduced pressure to give methyl 2-hydroxy[6-(trifluoromethyl)pyridin-2-yl]methylprop-2-enoate (2.7 g, 10.34 mmol, 54.9% yield) as brown oil.

### Step 5: Methyl 2-[(acetyloxy)[6-(trifluoromethyl)pyridin-2-yl]methyl]prop-2-enoate

Methyl 2-hydroxy[6-(trifluoromethyl)pyridin-2-yl]methylprop-2-enoate (2.7 g, 10.34 mmol) was dissolved in acetic anhydride (26.39 g, 258.46 mmol, 24.43 mL) and heated at 100° C. for 2 h. The reaction mixture was concentrated under reduced pressure, the residue was triturated with 100 mL of MTBE and the resulting mixture was quenched with sat. aq NaHCO₃. The organic phase was separated, washed with brine, dried over Na₂SO₄ and concentrated under reduced pressure to give methyl 2-[acetyloxy[6-(trifluoromethyl)pyridin-2-yl]methyl]prop-2-enoate (3.0 g, 9.89 mmol, 95.7% yield) as brown liquid.

### Step 6: Methyl 5-(trifluoromethyl)indolizine-2-carboxylate

Methyl 2-[acetyloxy)[6-(trifluoromethyl)pyridin-2-yl]methyl]prop-2-enoate (3.0 g, 9.89 mmol) was dissolved in xylene (70 mL) and refluxed for a week. The reaction mixture was cooled to r.t., diluted with MTBE (50 mL), quenched with NaHCO₃ aq, washed with brine, dried over Na₂SO₄ and concentrated under reduced pressure to give dark brown oil, which was purified by flash chromatography (Companion comb flash; SiO2 (120 g), petroleum ether/MTBE with MTBE from 0-12%, flow rate=85 mL/min, Rv=7 CV) to give methyl 5-(trifluoromethyl)indolizine-2-carboxylate (67.0 mg, 275.51 μmol, 2.8% yield) as light yellow crystals.

### Step 7: 5-(trifluoromethyl)indolizine-2-carboxylic Acid

To a solution of methyl 5-(trifluoromethyl)indolizine-2-carboxylate (67.0 mg, 275.51 μmol) in MeOH (4 mL) was added a solution of lithium hydroxide monohydrate (12.71 mg, 302.9 μmol) in 1 mL of H₂O. The resulting mixture was stirred at r.t. overnight. The reaction mixture was concentrated under reduced pressure and the residue was triturated with H₂O (15 mL). The resulting solution was acidified with 2N HCl to pH˜2 and extracted with MTBE (4×20 mL). The combined organic extracts were dried over Na₂SO₄ and concentrated in vacuo to give 5-(trifluoromethyl)indolizine-2-carboxylic acid (47.0 mg, 205.1 μmol, 74.5% yield) as pale yellow solid.

Rt (Method G) 1.10 mins, m/z 230 [M+H]⁺

### Synthesis of indolizine-2-carboxylic Acid

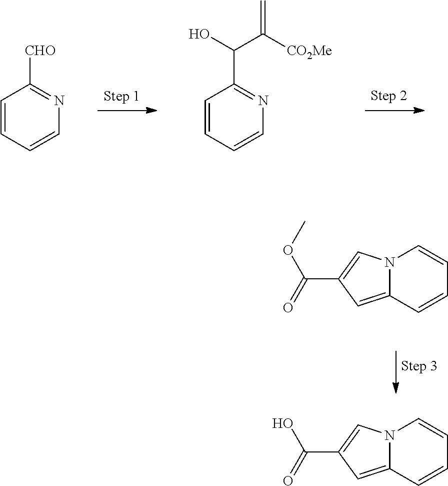

### Step 1: Methyl 2-(hydroxy(pyridin-2-yl)methyl)acrylate

Pyridine-2-carbaldehyde (0.85 g, 1 eq), methyl acrylate (3 eq) were dissolved in mixture of dioxane/H₂O (1/1) and stirred at room temperature in the presence of DABCO (1 eq). After the reaction was completed (as monitored by TLC), the mixture was diluted with MTBE and extracted twice. The combined organic layers were washed with brine, dried over Na₂SO₄ and solvents were removed under reduced pressure. The product was purified by column chromatography to give methyl 2-(hydroxy(pyridin-2-yl)methyl)acrylate (1 g, 65% yield).

### Step 2: Methyl indolizine-2-carboxylate

The reaction vessel was charged with methyl 2-(hydroxy(pyridin-2-yl)methyl)acrylate (0.74 g) and acetic anhydride. Then the reaction mixture was charged with Ar and heated under reflux for 4 hours. The cooled solution was poured onto ice with saturated NaHCO₃ aq solution and stirred for 1 hour, than resulted mixture was extracted with DCM (3×25 mL). The combined organic layers were dried over Na₂SO₄ and concentrated under reduced pressure. The product was purified by HPLC to give methyl indolizine-2-carboxylate. (0.2 g, 30% yield).

### Step 3: indolizine-2-carboxylic Acid

Methyl ester (0.164 g) was dissolved in MeOH/THF/H₂O (4/4/1) and NaOH (20% aq, 1.5 eq) was added. The obtained mixture was refluxed at 80° C. for 12 hours. Than the mixture was concentrated ½ under reduced pressure and resulting solution was acidified to pH=3-4 (with HCl 1N) at 0-5° C. The precipitate was filtered and dried to give indolizine-2-carboxylic acid (0.13 g, 86% yield)

Rt (Method G) 0.91 mins, m/z 160 [M−H]⁻

¹H NMR (400 MHz, DMSO-d6) δ 12.28 (s, 1H), 8.26 (d, J=7.1 Hz, 1H), 8.01 (d, J=1.6 Hz, 1H), 7.43 (d, J=9.1 Hz, 1H), 6.74 (dd, J=9.1, 6.5 Hz, 1H), 6.69 (s, 1H), 6.62 (t, J=6.7 Hz, 1H).

### Synthesis of 8-methylindolizine-2-carboxylic Acid

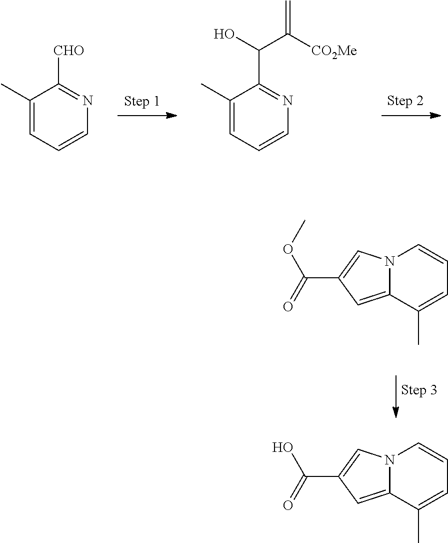

### Step 1: Methyl 2-[hydroxy(3-methylpyridin-2-yl)methyl]prop-2-enoate

Performed as described for indolizine-2-carboxylic acid, starting from 3-methylpyridine-2-carbaldehyde (58% yield).

### Step 2: methyl 2-[(acetyloxy)(3-methylpyridin-2-yl)methyl]prop-2-enoate

Performed as described for indolizine-2-carboxylic acid (42% yield)

### Step 3: 8-methylindolizine-2-carboxylic Acid

Performed as described for indolizine-2-carboxylic acid (83% yield)

Rt (Method G) 1.11 mins, m/z 174 [M−H]⁻

¹H NMR (400 MHz, DMSO-d6) δ 12.27 (s, 1H), 8.21-8.10 (m, 1H), 8.01 (s, 1H), 6.69 (s, 1H), 6.63-6.49 (m, 2H), 2.33 (s, 3H).

### Example 1

### 8-chloro-2-(3-{6,6-difluoro-4-azaspiro[2.4]heptane-4-carbonyl}-2H,4H,5H,6H,7H-pyrazolo[4,3-c]pyridine-5-carbonyl)indolizine

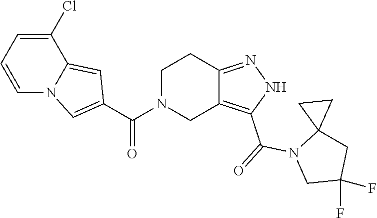 [SMILES: O=C(c1cc2c(Cl)cccn2c1)N1CCc2n[nH]c(C(=O)N3CC(F)(F)CC34CC4)c2C1]

Tert-butyl 3-(6,6-difluoro-4-azaspiro[2.4]heptane-4-carbonyl)-24(2-(trimethyl silyl)ethoxy)methyl)-2,4,6,7-tetrahydro-5H-pyrazolo[4,3-c]pyridine-5-carboxylate (0.298 g, 0.581 mmol) was dissolved in 4M HCl-dioxane (4 mL, 16.00 mmol) and stirred overnight. Further 4M HCl-dioxane (4 mL, 16.00 mmol) was added. After 6 h, the mixture was concentrated, the residue was treated with DIPEA (0.305 mL, 1.744 mmol) and ¼ of the mixture removed to a new vial. To this new vial was added a solution of 8-chloroindolizine-2-carboxylic acid (0.028 g, 0.145 mmol) and HATU (0.055 g, 0.145 mmol) in dry N,N-dimethylformamide (1 mL). The mixture was stirred at r.t. overnight, and then purified directly by preparative HPLC to give the desired product as a white solid (0.035 g, 0.076 mmol, 52% yield).

Rt (Method J) 1.45 mins, m/z 460 [M+H]⁺

1H NMR (400 MHz, DMSO-d6) δ 13.14 (s, 1H), 8.31 (d, J=7.0 Hz, 1H), 8.00 (m, 1H), 6.98 (d, J=7.1 Hz, 1H), 6.66 (m, 2H), 4.72 (m, 2H), 4.46 (t, J=13.6 Hz, 2H), 3.85 (t, J=5.7 Hz, 2H), 2.82 (s, 2H), 2.46 (m, 2H), 1.92 (m, 2H), 0.63 (m, 2H).

### Example 2

### 6,8-difluoro-2-(3-{6,6-difluoro-4-azaspiro[2.4]heptane-4-carbonyl}-2H,4H,5H,6H,7H-pyrazolo[4,3-c]pyridine-5-carbonyl)indolizine

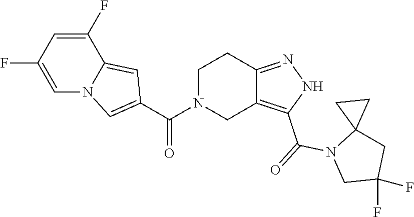 [SMILES: O=C(c1cc2c(F)cc(F)cn2c1)N1CCc2n[nH]c(C(=O)N3CC(F)(F)CC34CC4)c2C1]

Tert-butyl 3-(6,6-difluoro-4-azaspiro[2.4]heptane-4-carbonyl)-2-((2-(trimethyl silyl)ethoxy)methyl)-2,4,6,7-tetrahydro-5H-pyrazolo[4,3-c]pyridine-5-carboxylate (0.298 g, 0.581 mmol) was dissolved in 4M HCl-dioxane (4 mL, 16.00 mmol) and stirred overnight. Further 4M HCl-dioxane (4 mL, 16.00 mmol) was added. After 6 h, the mixture was concentrated, the residue was treated with DIPEA (0.305 mL, 1.744 mmol) and ¼ of the mixture removed to a new vial. To this new vial was added a solution of 6,8-difluoro-indolizine-2-carboxylic acid (0.029 g, 0.145 mmol) and HATU (0.055 g, 0.145 mmol) in dry N,N-dimethylformamide (1 mL). The mixture was stirred at r.t. overnight, and then purified directly by preparative HPLC to give the desired product as a white solid (0.024 g, 0.052 mmol, 36% yield).

Rt (Method J) 1.43 mins, m/z 462 [M+H]⁺

1H NMR (400 MHz, DMSO-d6) δ 13.14 (s, 0H), 8.53-8.36 (m, 1H), 8.08-7.88 (m, 1H), 7.06-6.90 (m, 1H), 6.81 (s, 1H), 5.01-4.56 (m, 2H), 4.55-4.32 (m, 2H), 3.93-3.71 (m, 2H), 2.82 (s, 2H), 2.05-1.68 (m, 2H), 0.77-0.49 (m, 2H).

### Example 3

### 5-(8-chloroindolizine-2-carbonyl)-N-[1-(methoxymethyl)cyclopropyl]-N-methyl-2H,4H,5H,6H,7H-pyrazolo[4,3-c]pyridine-3-carboxamide

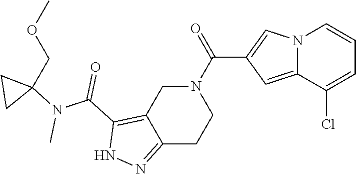 [SMILES: COCC1(N(C)C(=O)c2[nH]nc3c2CN(C(=O)c2cc4c(Cl)cccn4c2)CC3)CC1]

To a solution of 8-chloroindolizine-2-carboxylic acid (11.0 mg, 0.056 mmol) in dry N,N-dimethylformamide (0.4 mL) was added HATU (25.7 mg, 0.068 mmol). In a separate vial N-(1-(methoxymethyl)cyclopropyl)-N-methyl-4,5,6,7-tetrahydro-1H-pyrazolo[4,3-c]pyridine-3-carboxamide dihydrochloride (19 mg, 0.056 mmol) was dissolved in dry N,N-dimethylformamide (0.400 mL) and TEA (0.039 mL, 0.282 mmol) was added. After 5 minutes the flasks were combined and stirred overnight. A few drops of water were added, and the mixture was purified directly by chromatography to give the desired product (0.0094 g, 0.021 mmol, 38% yield).

Rt (Method A) 3.11 mins, m/z 442/444 [M+H]⁺

1H NMR (400 MHz, DMSO-d6) δ 13.19-12.81 (m, 1H), 8.31 (d, J=6.8 Hz, 1H), 8.00 (s, 1H), 6.98 (d, J=7.2 Hz, 1H), 6.69-6.62 (m, 2H), 4.96-4.51 (m, 2H), 3.96-3.80 (m, 2H), 3.63-3.38 (m, 1H), 3.30-3.16 (m, 5H), 3.12-2.71 (m, 4H), 0.92-0.63 (m, 4H).

### Example 4

### 5-(5-chloroindolizine-2-carbonyl)-N-[1-(methoxymethyl)cyclopropyl]-N-methyl-2H,4H,5H,6H,7H-pyrazolo[4,3-c]pyridine-3-carboxamide

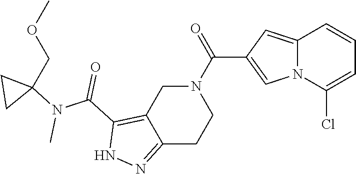 [SMILES: COCC1(N(C)C(=O)c2[nH]nc3c2CN(C(=O)c2cc4cccc(Cl)n4c2)CC3)CC1]

To a solution of 5-chloroindolizine-2-carboxylic acid (11.0 mg, 0.056 mmol) in dry N,N-dimethylformamide (0.5 mL) was added HATU (25.7 mg, 0.068 mmol). In a separate vial N-(1-(methoxymethyl)cyclopropyl)-N-methyl-4,5,6,7-tetrahydro-1H-pyrazolo[4,3-c]pyridine-3-carboxamide dihydrochloride (19 mg, 0.056 mmol) was dissolved in dry N,N-dimethylformamide (0.500 mL) and TEA (0.039 mL, 0.282 mmol) was added. After 5 minutes the flasks were combined and stirred overnight. A few drops of water were added, and the mixture was purified directly by chromatography to give the desired product (0.0137 g, 0.031 mmol, 55% yield).

Rt (Method A) 3.10 mins, m/z 442/444 [M+H]⁺

1H NMR (400 MHz, DMSO-d6) δ 13.14-12.85 (m, 1H), 7.84 (s, 1H), 7.57 (d, J=8.9 Hz, 1H), 6.98 (d, J=6.9 Hz, 1H), 6.89-6.79 (m, 2H), 4.96-4.49 (m, 2H), 3.99-3.74 (m, 2H), 3.58-3.38 (m, 1H), 3.30-3.13 (m, 5H), 3.10-2.69 (m, 4H), 0.94-0.58 (m, 4H).

### Example 5

### 5-(6,8-difluoroindolizine-2-carbonyl)-N-[1-(methoxymethyl)cyclopropyl]-N-methyl-2H,4H,5H,6H,7H-pyrazolo[4,3-c]pyridine-3-carboxamide

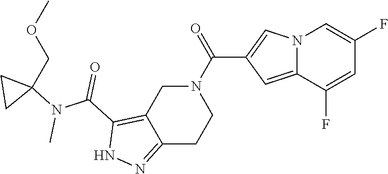 [SMILES: COCC1(N(C)C(=O)c2[nH]nc3c2CN(C(=O)c2cc4c(F)cc(F)cn4c2)CC3)CC1]

To a solution of 6,8-difluoroindolizine-2-carboxylic acid (11.1 mg, 0.056 mmol) in dry N,N-dimethylformamide (0.5 mL) was added HATU (25.7 mg, 0.068 mmol). In a separate vial N-(1-(methoxymethyl)cyclopropyl)-N-methyl-4,5,6,7-tetrahydro-1H-pyrazolo[4,3-c]pyridine-3-carboxamide dihydrochloride (19 mg, 0.056 mmol) was dissolved in dry N,N-dimethylformamide (0.500 mL) and TEA (0.039 mL, 0.282 mmol) was added. After 5 minutes the flasks were combined and stirred overnight. A few drops of water were added, and the mixture was purified directly by chromatography to give the desired product (0.0137 g, 0.031 mmol, 55% yield).

Rt (Method A) 3.09 mins, m/z 442/444 [M+H]⁺

1H NMR (400 MHz, DMSO-d6) δ 13.14-12.77 (m, 1H), 8.46 (s, 1H), 8.01 (s, 1H), 7.07-6.96 (m, 1H), 6.81 (s, 1H), 5.06-4.45 (m, 2H), 3.95-3.76 (m, 2H), 3.60-3.38 (m, 1H), 3.25 (s, 5H), 3.10-2.70 (m, 4H), 0.94-0.47 (m, 4H).

### Example 6

### 5-(8-chloroindolizine-2-carbonyl)-N-{1-[(difluoromethoxy)methyl]cyclopropyl}-N-methyl-4H,5H,6H,7H-pyrazolo[1,5-a]pyrazine-3-carboxamide

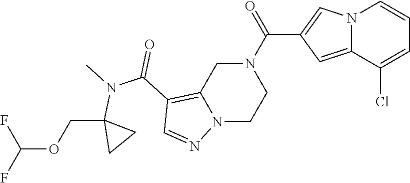 [SMILES: CN(C(=O)c1cnn2c1CN(C(=O)c1cc3c(Cl)cccn3c1)CC2)C1(COC(F)F)CC1]

To a solution of 8-chloroindolizine-2-carboxylic acid (17.4 mg, 0.089 mmol) in DMSO (0.4 mL) was added HATU (37.3 mg, 0.098 mmol). Triethylamine (0.05 mL, 0.359 mmol) was added, followed by a solution of N-(1-((difluoromethoxy)methyl)cyclopropyl)-N-methyl-4,5,6,7-tetrahydropyrazolo[1,5-a]pyrazine-3-carboxamide hydrochloride (30 mg, 0.089 mmol) in DMSO (0.4 mL). The mixture was stirred overnight. The mixture was filtered, and flushed with methanol (0.1 mL). The filtrate was purified directly by chromatography to give the desired product (0.0249 g, 0.052 mmol, 58% yield).

Rt (Method B) 3.21 mins, m/z 478/480 [M+H]⁺

1H NMR (400 MHz, DMSO-d6) δ 8.32 (d, J=6.9 Hz, 1H), 8.05 (s, 1H), 7.83 (s, 1H), 7.00 (d, J=7.1 Hz, 1H), 6.92-6.46 (m, 3H), 5.30-4.83 (m, 2H), 4.39-3.80 (m, 6H), 3.23-2.80 (m, 3H), 1.31-0.75 (m, 4H).

### Example 7

### 5-(6,8-difluoroindolizine-2-carbonyl)-N-{1-[(difluoromethoxy)methyl]cyclopropyl}-N-methyl-4H,5H,6H,7H-pyrazolo[1,5-a]pyrazine-3-carboxamide

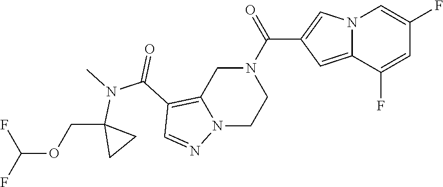 [SMILES: CN(C(=O)c1cnn2c1CN(C(=O)c1cc3c(F)cc(F)cn3c1)CC2)C1(COC(F)F)CC1]

To 6,8-difluoroindolizine-2-carboxylic acid (17.5 mg, 0.089 mmol) was added a solution of HATU (37.3 mg, 0.098 mmol) in DMSO (0.4 mL). The mixture was stirred for 10 mins, then triethylamine (0.05 mL, 0.359 mmol) was added, followed by a solution of N-(1-((difluoromethoxy)methyl)cyclopropyl)-N-methyl-4,5,6,7-tetrahydropyrazolo[1,5-a]pyrazine-3-carboxamide hydrochloride (30 mg, 0.089 mmol) in DMSO (0.4 mL). The mixture was stirred overnight. The mixture was filtered, and flushed with methanol (0.1 mL). The filtrate was purified directly by chromatography to give the desired product (0.0238 g, 0.052 mmol, 58% yield).

Rt (Method B) 3.18 mins, m/z 480 [M+H]⁺

1H NMR (400 MHz, DMSO-d6) δ 8.50-8.44 (m, 1H), 8.06 (s, 1H), 7.83 (s, 1H), 7.07-6.98 (m, 1H), 6.93-6.43 (m, 2H), 5.33-4.79 (m, 2H), 4.38-4.18 (m, 2H), 4.18-3.89 (m, 4H), 3.21-2.87 (m, 3H), 1.37-0.66 (m, 4H).

### Example 8

### 5-(5-chloroindolizine-2-carbonyl)-N-{1-[(difluoromethoxy)methyl]cyclopropyl}-N-methyl-4H,5H,6H,7H-pyrazolo[1,5-a]pyrazine-3-carboxamide

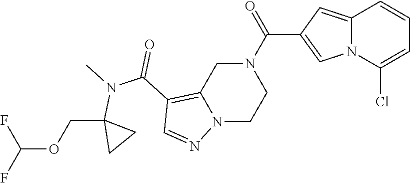 [SMILES: CN(C(=O)c1cnn2c1CN(C(=O)c1cc3cccc(Cl)n3c1)CC2)C1(COC(F)F)CC1]

To 5-chloroindolizine-2-carboxylic acid (17.4 mg, 0.089 mmol) was added a solution of HATU (37.3 mg, 0.098 mmol) in DMSO (0.4 mL). The mixture was stirred for 10 mins, then triethylamine (0.05 mL, 0.359 mmol) was added, followed by a solution of N-(1-((difluoromethoxy)methyl)cyclopropyl)-N-methyl-4,5,6,7-tetrahydropyrazolo[1,5-a]pyrazine-3-carboxamide hydrochloride (30 mg, 0.089 mmol) in DMSO (0.4 mL). The mixture was stirred overnight. The mixture was filtered, and flushed with methanol (0.1 mL). The filtrate was purified directly by chromatography to give the desired product (0.0241 g, 0.050 mmol, 57% yield).

Rt (Method B) 3.20 mins, m/z 478/480 [M+H]⁺

1H NMR (400 MHz, DMSO-d6) δ 7.90 (s, 1H), 7.83 (s, 1H), 7.59 (d, J=8.9 Hz, 1H), 6.99 (d, J=6.7 Hz, 1H), 6.92-6.42 (m, 3H), 5.25-4.88 (m, 2H), 4.31-4.21 (m, 2H), 4.18-3.91 (m, 4H), 3.20-2.88 (m, 3H), 1.17-0.76 (m, 4H)

### Example 9

### 5-(8-chloroindolizine-2-carbonyl)-N-{1-[(difluoromethoxy)methyl]cyclopropyl}-N,6-dimethyl-4H,5H,6H,7H-pyrazolo[1,5-a]pyrazine-3-carboxamide

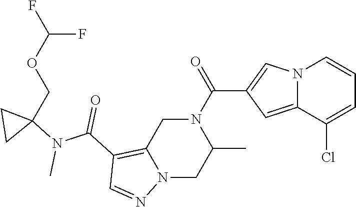 [SMILES: CC1Cn2ncc(C(=O)N(C)C3(COC(F)F)CC3)c2CN1C(=O)c1cc2c(Cl)cccn2c1]

To 8-chloroindolizine-2-carboxylic acid (17.4 mg, 0.089 mmol) was added a solution of HATU (37.2 mg, 0.098 mmol) in DMSO (0.4 mL). The mixture was stirred for 10 mins, then triethylamine (0.05 mL, 0.359 mmol) was added, followed by a solution of N-(1-((difluoromethoxy)methyl)cyclopropyl)-N,6-dimethyl-4,5,6,7-tetrahydropyrazolo[1,5-a]pyrazine-3-carboxamide hydrochloride (31.2 mg, 0.089 mmol) in DMSO (0.4 mL). The mixture was stirred overnight. The mixture was filtered, and flushed with methanol (0.1 mL). The filtrate was purified directly by chromatography to give the desired product (0.0213 g, 0.043 mmol, 49% yield).

Rt (Method B) 3.29 mins, m/z 492/494 [M+H]⁺

1H NMR (400 MHz, DMSO-d6) δ 8.32 (d, J=7.0 Hz, 1H), 8.04 (d, J=1.5 Hz, 1H), 7.86 (s, 1H), 7.00 (d, J=7.1 Hz, 1H), 6.93-6.46 (m, 3H), 5.60-5.35 (m, 1H), 5.19-4.95 (m, 1H), 4.76-4.41 (m, 1H), 4.41-4.26 (m, 1H), 4.18-3.89 (m, 3H), 3.20-2.92 (m, 3H), 1.20 (d, J=6.6 Hz, 3H), 1.15-0.70 (m, 4H).

### Example 10

### 8-chloro-2-[6-methyl-3-(1,3-thiazol-4-yl)-1H,4H,5H,6H,7H-pyrazolo[4,3-c]pyridine-5-carbonyl]indolizine

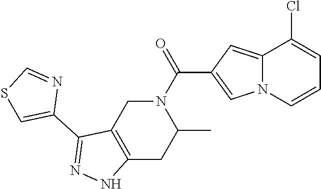 [SMILES: CC1Cc2[nH]nc(-c3cscn3)c2CN1C(=O)c1cc2c(Cl)cccn2c1]

Rt (Method B2) 3.38 mins, m/z 398/400 [M+H]⁺

1H NMR (400 MHz, DMSO-d6) δ 12.95 (s, 1H), 9.22 (s, 1H), 8.31 (d, J=7.0 Hz, 1H), 8.03-7.97 (m, 1H), 7.90 (s, 1H), 6.98 (d, J=7.1 Hz, 1H), 6.70-6.62 (m, 2H), 5.60-5.31 (m, 1H), 5.15-4.05 (m, 2H), 3.12-2.97 (m, 1H), 2.66-2.59 (m, 1H), 1.19 (d, J=6.7 Hz, 3H).

### Example 11

### 1-bromo-2-[6-methyl-3-(1,3-thiazol-4-yl)-1H,4H,5H,6H,7H-pyrazolo[4,3-c]pyridine-5-carbonyl]indolizine

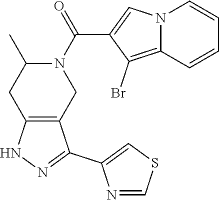 [SMILES: CC1Cc2[nH]nc(-c3cscn3)c2CN1C(=O)c1cn2ccccc2c1Br]

Rt (Method B2) 3.35 mins, m/z 442/444 [M+H]⁺

1H NMR (400 MHz, DMSO-d6) δ 13.32-12.72 (m, 1H), 9.37-8.99 (m, 1H), 8.31 (d, J=7.0 Hz, 1H), 8.05-7.80 (m, 2H), 7.36 (d, J=9.1 Hz, 1H), 6.97-6.88 (m, 1H), 6.72 (t, J=6.8 Hz, 1H), 5.68-4.09 (m, 3H), 3.06-2.87 (m, 1H), 2.61-2.54 (m, 1H), 1.16 (s, 3H).

### Example 12

### 2-[6-methyl-3-(1,3-thiazol-4-yl)-1H,4H,5H,6H,7H-pyrazolo[4,3-c]pyridine-5-carbonyl]-5-(trifluoromethyl)indolizine

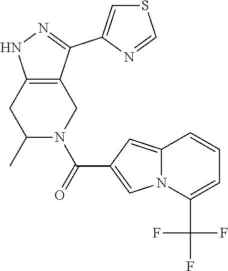 [SMILES: CC1Cc2[nH]nc(-c3cscn3)c2CN1C(=O)c1cc2cccc(C(F)(F)F)n2c1]

Rt (Method B2) 3.52 mins, m/z 432 [M+H]⁺

1H NMR (400 MHz, DMSO-d6) δ 12.94 (s, 1H), 9.23 (s, 1H), 7.99-7.74 (m, 3H), 7.36 (d, J=6.9 Hz, 1H), 7.01-6.88 (m, 2H), 5.64-5.19 (m, 1H), 4.90-4.64 (m, 1H), 4.31-4.09 (m, 1H), 3.10-2.99 (m, 1H), 2.65-2.59 (m, 1H), 1.28-1.10 (m, 3H).

### Example 13—Intentionally Left Blank

### Example 14—Intentionally Left Blank

### Example 15—Intentionally Left Blank

### Example 16

### 5-methyl-2-[6-methyl-3-(1,3-thiazol-4-yl)-1H,4H,5H,6H,7H-pyrazolo[4,3-c]pyridine-5-carbonyl]indolizine

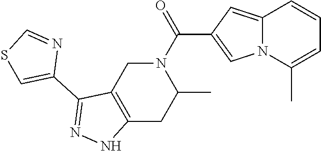 [SMILES: Cc1cccc2cc(C(=O)N3Cc4c(-c5cscn5)n[nH]c4CC3C)cn12]

Rt (Method B2) 3.30 mins, m/z 378 [M+H]⁺

### Example 17

### 8-methyl-2-[6-methyl-3-(1,3-thiazol-4-yl)-1H,4H,5H,6H,7H-pyrazolo[4,3-c]pyridine-5-carbonyl]indolizine

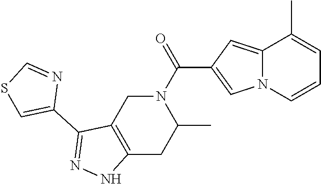 [SMILES: Cc1cccn2cc(C(=O)N3Cc4c(-c5cscn5)n[nH]c4CC3C)cc12]

Rt (Method B2) 3.32 mins, m/z 378 [M+H]⁺

### Example 18

### 6,8-dichloro-2-[6-methyl-3-(1,3-thiazol-4-yl)-1H,4H,5H,6H,7H-pyrazolo[4,3-c]pyridine-5-carbonyl]indolizine

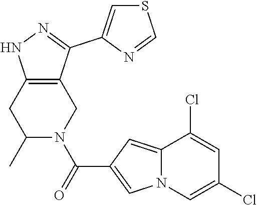 [SMILES: CC1Cc2[nH]nc(-c3cscn3)c2CN1C(=O)c1cc2c(Cl)cc(Cl)cn2c1]

Rt (Method B2) 3.73 mins, m/z 432/434 [M+H]⁺

### Example 19

### 7-chloro-2-[6-methyl-3-(1,3-thiazol-4-yl)-1H,4H,5H,6H,7H-pyrazolo[4,3-c]pyridine-5-carbonyl]indolizine

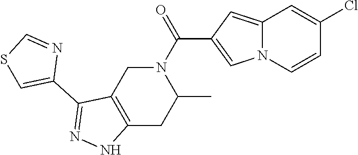 [SMILES: CC1Cc2[nH]nc(-c3cscn3)c2CN1C(=O)c1cc2cc(Cl)ccn2c1]

Rt (Method B2) 3.45 mins, m/z 398/400 [M+H]⁺

### Example 20

### 5-chloro-2-[6-methyl-3-(1,3-thiazol-4-yl)-1H,4H,5H,6H,7H-pyrazolo[4,3-c]pyridine-5-carbonyl]indolizine

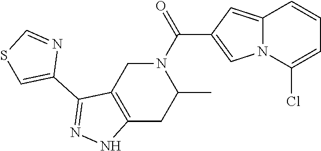 [SMILES: CC1Cc2[nH]nc(-c3cscn3)c2CN1C(=O)c1cc2cccc(Cl)n2c1]

Rt (Method B2) 3.40 mins, m/z 398/400 [M+H]⁺

1H NMR (400 MHz, DMSO-d6) δ 13.37-12.62 (m, 1H), 9.23 (s, 1H), 7.99-7.78 (m, 2H), 7.57 (d, J=8.9 Hz, 1H), 7.03-6.94 (m, 1H), 6.91-6.79 (m, 2H), 5.68-5.21 (m, 1H), 5.13-4.00 (m, 2H), 3.16-2.94 (m, 1H), 2.66-2.57 (m, 1H), 1.29-1.08 (m, 3H).

### Example 21

### 6-chloro-2-[6-methyl-3-(1,3-thiazol-4-yl)-1H,4H,5H,6H,7H-pyrazolo[4,3-c]pyridine-5-carbonyl]indolizine

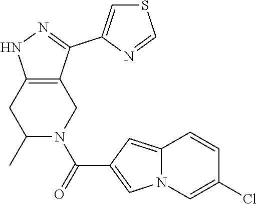 [SMILES: CC1Cc2[nH]nc(-c3cscn3)c2CN1C(=O)c1cc2ccc(Cl)cn2c1]

Rt (Method B2) 3.42 mins, m/z 398/400 [M+H]⁺

### Example 22

### 7-chloro-6-fluoro-2-[6-methyl-3-(1,3-thiazol-4-yl)-1H,4H,5H,6H,7H-pyrazolo[4,3-c]pyridine-5-carbonyl]indolizine

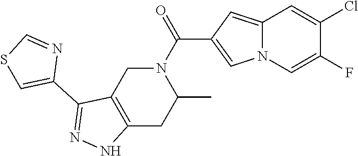 [SMILES: CC1Cc2[nH]nc(-c3cscn3)c2CN1C(=O)c1cc2cc(Cl)c(F)cn2c1]

Rt (Method B2) 3.49 mins, m/z 416/418 [M+H]⁺

### Example 23

### 6-fluoro-2-[6-methyl-3-(1,3-thiazol-4-yl)-1H,4H,5H,6H,7H-pyrazolo[4,3-c]pyridine-5-carbonyl]indolizine

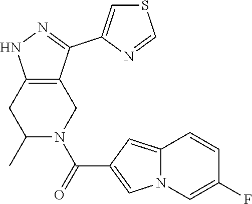 [SMILES: CC1Cc2[nH]nc(-c3cscn3)c2CN1C(=O)c1cc2ccc(F)cn2c1]

Rt (Method B2) 3.21 mins, m/z 382 [M+H]⁺

### Example 24

### 8-fluoro-2-[6-methyl-3-(1,3-thiazol-4-yl)-1H,4H,5H,6H,7H-pyrazolo[4,3-c]pyridine-5-carbonyl]indolizine

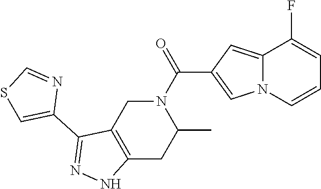 [SMILES: CC1Cc2[nH]nc(-c3cscn3)c2CN1C(=O)c1cc2c(F)cccn2c1]

Rt (Method B2) 3.24 mins, m/z 382 [M+H]⁺

### Example 25

### 2-[6-methyl-3-(1,3-thiazol-4-yl)-1H,4H,5H,6H,7H-pyrazolo[4,3-c]pyridine-5-carbonyl]-8-(trifluoromethyl)indolizine

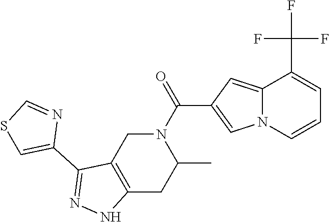 [SMILES: CC1Cc2[nH]nc(-c3cscn3)c2CN1C(=O)c1cc2c(C(F)(F)F)cccn2c1]

Rt (Method B2) 3.51 mins, m/z 432 [M+H]⁺

### Example 26

### 2-[6-methyl-3-(1,3-thiazol-4-yl)-1H,4H,5H,6H,7H-pyrazolo[4,3-c]pyridine-5-carbonyl]indolizine

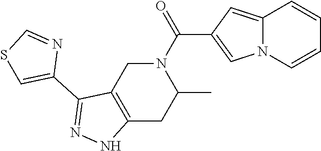 [SMILES: CC1Cc2[nH]nc(-c3cscn3)c2CN1C(=O)c1cc2ccccn2c1]

Rt (Method B2) 3.12 mins, m/z 364 [M+H]⁺

### Example 27

### 5-(indolizine-2-carbonyl)-N-[1-(trifluoromethyl)cyclobutyl]-2H,4H,5H,6H,7H-pyrazolo[4,3-c]pyridine-3-carboxamide

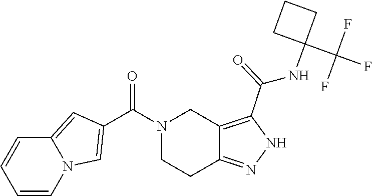 [SMILES: O=C(NC1(C(F)(F)F)CCC1)c1[nH]nc2c1CN(C(=O)c1cc3ccccn3c1)CC2]

LCMS (ESI): [M+H]⁺ m/z: calcd 432.18; found 432.1; Rt=1.303 min.

### Example 28

### 2-[3-(1H-1,2,3-triazol-1-yl)-1H,4H,5H,6H,7H-pyrazolo[4,3-c]pyridine-5-carbonyl]indolizine

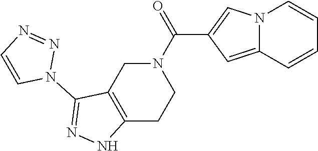 [SMILES: O=C(c1cc2ccccn2c1)N1CCc2[nH]nc(-n3ccnn3)c2C1]

LCMS (ESI): [M+H]⁺ m/z: calcd 334.1; found 334.0; Rt=0.916 min.

### Example 29

### 5-(indolizine-2-carbonyl)-N-[(oxolan-2-yl)methyl]-2H,4H,5H,6H,7H-pyrazolo[4,3-c]pyridine-3-carboxamide

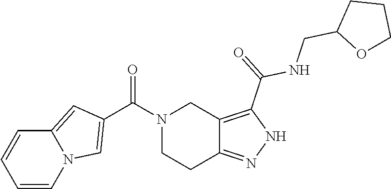 [SMILES: O=C(NCC1CCCO1)c1[nH]nc2c1CN(C(=O)c1cc3ccccn3c1)CC2]

LCMS (ESI): [M+H]⁺ m/z: calcd 394.2; found 394.2; Rt=2.915 min.

### Example 30

### 5-(indolizine-2-carbonyl)-N-[(oxan-3-yl)methyl]-2H,4H,5H,6H,7H-pyrazolo[4,3-c]pyridine-3-carboxamide

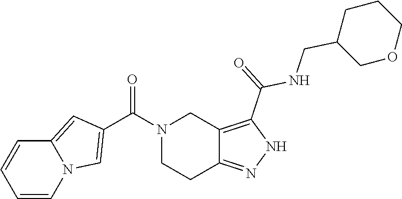 [SMILES: O=C(NCC1CCCOC1)c1[nH]nc2c1CN(C(=O)c1cc3ccccn3c1)CC2]

LCMS (ESI): [M+H]⁺ m/z: calcd 408.2; found 408.2; Rt=2.319 min.

### Example 31

### 2-[3-(1,2-oxazol-3-yl)-1H,4H,5H,6H,7H-pyrazolo[4,3-c]pyridine-5-carbonyl]indolizine

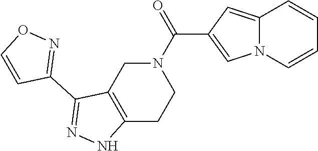 [SMILES: O=C(c1cc2ccccn2c1)N1CCc2[nH]nc(-c3ccon3)c2C1]

LCMS (ESI): [M+H]⁺ m/z: calcd 334.1; found 334.2; Rt=2.191 min.

### Example 32

### 5-(indolizine-2-carbonyl)-N-[3-(trifluoromethyl)cyclobutyl]-2H,4H,5H,6H,7H-pyrazolo[4,3-c]pyridine-3-carboxamide

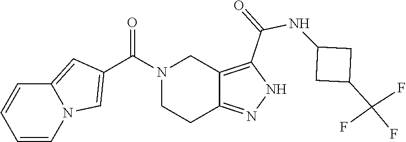 [SMILES: O=C(NC1CC(C(F)(F)F)C1)c1[nH]nc2c1CN(C(=O)c1cc3ccccn3c1)CC2]

LCMS (ESI): [M+H]⁺ m/z: calcd 432.2; found 432.2; Rt=1.252 min.

### Example 33

### 5-(indolizine-2-carbonyl)-N-[1-(trifluoromethyl)cyclopropyl]-2H,4H,5H,6H,7H-pyrazolo[4,3-c]pyridine-3-carboxamide

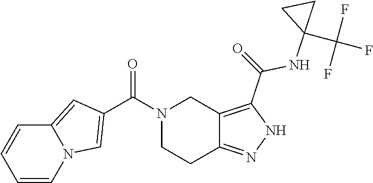 [SMILES: O=C(NC1(C(F)(F)F)CC1)c1[nH]nc2c1CN(C(=O)c1cc3ccccn3c1)CC2]

LCMS (ESI): [M+H]⁺ m/z: calcd 418.2; found 418.2; Rt=2.670 min.

### Example 34

### 2-[3-(1,3-thiazol-4-yl)-4H,5H,6H,7H-[1,2]oxazolo[4,3-c]pyridine-5-carbonyl]indolizine

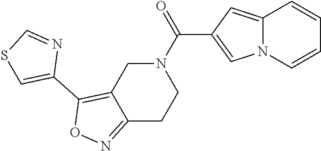 [SMILES: O=C(c1cc2ccccn2c1)N1CCc2noc(-c3cscn3)c2C1]

LCMS (ESI): [M+H]⁺ m/z: calcd 351.1; found 351.2; Rt=2.775 min.

### Example 35

### 5-(indolizine-2-carbonyl)-N-(1,1,1-trifluoro-2-methylpropan-2-yl)-2H,4H,5H,6H,7H-pyrazolo[4,3-c]pyridine-3-carboxamide

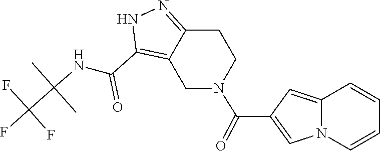 [SMILES: CC(C)(NC(=O)c1[nH]nc2c1CN(C(=O)c1cc3ccccn3c1)CC2)C(F)(F)F]

LCMS (ESI): [M+H]⁺ m/z: calcd 420.2; found 420.2; Rt=1.233 min.

### Example 36

### 2-[3-(1,3-thiazol-4-yl)-4H,5H,6H,7H-[1,2]oxazolo[4,5-c]pyridine-5-carbonyl]indolizine

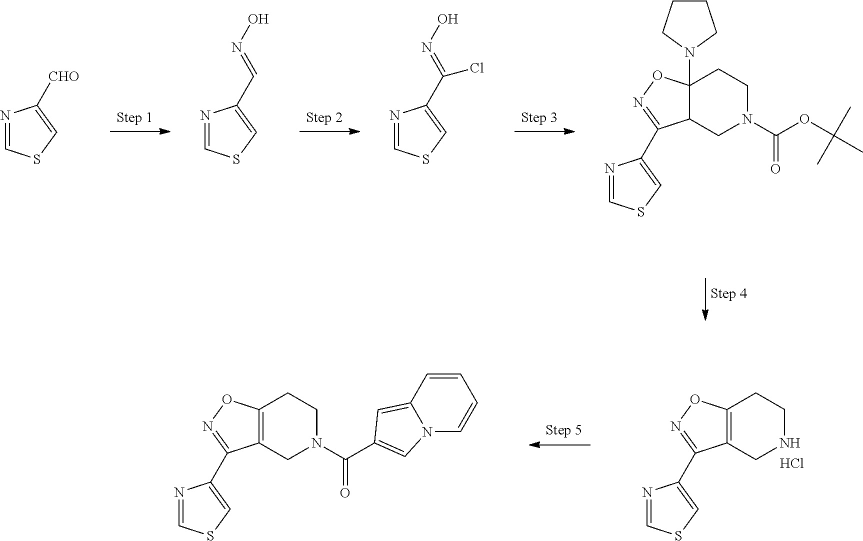

### Step 1

To a stirred solution of 1,3-thiazole-4-carbaldehyde (3.0 g, 26.52 mmol) in EtOH (20 mL) was added hydroxylamine hydrochloride (2.03 g, 29.18 mmol), followed by pyridine (2.31 g, 29.18 mmol, 2.36 mL, 1.1 equiv.). The reaction mixture was stirred for 3 h at rt, quenched with sat. aq. NH₄Cl, then extracted with EtOAc. The organic layer was separated, dried over Na₂SO₄, and concentrated in vacuo to afford (E)-N-[1,3-thiazol-4-yl)methylidene]hydroxylamine (2.4 g, 18.73 mmol, 70.6% yield) as yellow solid

¹H NMR (500 MHz, DMSO-d6) 7.736 (s, 1H), 8.229 (s, 1H), 8.558 (s, 1H), 11.331 (bds, 1H), 12.047 (bds, 1H).

### Step 2

To a cooled (0° C.) solution of N-(1,3-thiazol-4-ylmethylidene)hydroxylamine (2.4 g, 18.73 mmol) in DMF (20 mL) was added portionwise 1-chloropyrrolidine-2,5-dione (2.63 g, 19.66 mmol). The mixture was stirred at r.t. for 2 hr, then diluted with water (30 mL). The precipitate formed was collected by filtration, washed with water (10 mL) and dried to afford (Z)-N-hydroxy-1,3-thiazole-4-carbonimidoyl chloride (2.5 g, 15.38 mmol, 82.1% yield) as a beige solid.

¹H NMR (500 MHz, DMSO-d₆) 8.141 (s, 1H), 9.173 (s, 1H), 12.447 (bds, 1H).

### Step 3

To a cooled (0° C.) solution of tert-butyl 4-(pyrrolidin-1-yl)-1,2,3,6-tetrahydropyridine-1-carboxylate (1.19 g, 4.73 mmol) in DCM (25 mL) was added (Z)-N-hydroxy-1,3-thiazole-4-carbonimidoyl chloride (1.0 g, 6.15 mmol), followed by triethylamine (1.44 g, 14.2 mmol). The reaction mixture was stirred at r.t. overnight, then diluted with DCM and washed with saturated aq. sodium bicarbonate. The organic phase was dried over sodium sulfate, filtered and concentrated in vacuo to afford crude tert-butyl 7a-(pyrrolidin-1-yl)-3-(1,3-thiazol-4-yl)-3 aH,4H,5H,6H,7H,7aH-[1,2]oxazolo[4,5-c]pyridine-5-carboxylate (1.55 g, 80.0% purity, 3.28 mmol, 69.2% yield) as a brown oil.

LCMS (ESI): [M+H]⁺ m/z: calcd 379.2; found 379.2; Rt=0.844 min.

### Step 4

To a solution of tert-butyl tert-butyl 7a-(pyrrolidin-1-yl)-3-(1,3-thiazol-4-yl)-3aH,4H,5H,6H,7H,7aH-[1,2]oxazolo[4,5-c]pyridine-5-carboxylate (1.35 g, 3.57 mmol) in EtOH (5 mL) was added 12N HCl (5 mL). The reaction mixture was stirred at 80° C. for 10 h, then concentrated in vacuo. The residue was dried under vacuum to afford crude 4-4H,5H,6H,7H-[1,2]oxazolo[4,5-c]pyridin-3-yl-1,3-thiazole hydrochloride (860.0 mg, 3.53 mmol, 99% yield) as a brown solid.

¹H NMR (500 MHz, DMSO-d₆) 3.103 (m, 2H), 3.428 (m, 2H), 4.250 (m, 2H), 8.341 (s, 1H), 9.396 (s, 1H), 10.148 (bds, 1H).

### Step 5

To a solution of indolizine-2-carboxylic acid (164.2 mg, 1.02 mmol) in DMF (2 mL) was added [(dimethylamino)(3H-[1,2,3]triazolo[4,5-b]pyridin-3-yloxy)methylidene]dimethylazanium; hexafluoro-lambda5-phosphanuide (387.4 mg, 1.02 mmol). The mixture was stirred for 10 min, then 4-4H,5H,6H,7H-[1,2]oxazolo[4,5-c]pyridin-3-yl-1,3-thiazole hydrochloride (248.31 mg, 1.02 mmol) and triethylamine (515.46 mg, 5.09 mmol, 710.0 μl, 5.0 equiv) were added. The reaction mixture was stirred at r.t. overnight. The product was purified directly by HPLC to afford 2-[3-(1,3-thiazol-4-yl)-4H,5H,6H,7H-[1,2]oxazolo[4,5-c]pyridine-5-carbonyl]indolizine (39.0 mg, 111.3 μmol, 10.9% yield) as yellow solid.

LCMS (ESI): [M+H]⁺ m/z: calcd 351.09; found 351.2; Rt=2.917 min.

1H NMR (400 MHz, DMSO) δ 9.33 (br s, 1H), 8.33 (s, 1H), 8.26 (d, J=6.9 Hz, 1H), 7.89 (s, 1H), 7.45 (d, J=9.0 Hz, 1H), 6.76 (m, 1H), 6.63 (m, 2H), 4.86 (m, 2H), 4.00 (m, 2H), 3.02 (m, 2H).

### Example 37

### 2-[3-(1,3-thiazol-4-yl)-1H,4H,5H,6H,7H-pyrazolo[4,3-c]pyridine-5-carbonyl]indolizine

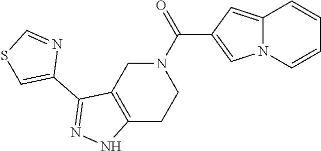 [SMILES: O=C(c1cc2ccccn2c1)N1CCc2[nH]nc(-c3cscn3)c2C1]

### Step 1

To a solution of tert-butyl 4-oxo-3-(1,3-thiazole-4-carbonyl)piperidine-1-carboxylate (1.5 g, 4.83 mmol) in EtOH (10 mL) were added hydrazine hydrate (524.3 mg, 10.47 mmol, 520.0 μL, 1.3 equiv) and acetic acid (464.45 mg, 7.73 mmol, 450.0 μL, 1.6 equiv). The reaction mixture was refluxed for 5 h, cooled and concentrated. The residue was dissolved in EtOAc (20 mL). The solution was washed with sat. aq. sodium bicarbonate, dried over sodium sulfate, filtered and concentrated. The residue was purified by column chromatography (silica, MTBE-hexane gradient) to afford tert-butyl 3-(1,3-thiazol-4-yl)-1H,4H,5H,6H,7H-pyrazolo[4,3-c]pyridine-5-carboxylate (180.0 mg, 587.5 μmol, 12.2% yield) as a yellow solid

¹H NMR (500 MHz, CDCl₃) 1.513 (s, 9H), 2.844 (m, 2H), 3.768 (m, 2H), 4.715 (m, 2H), 7.426 (s, 1H), 8.937 (s, 1H).

LCMS(ESI): [M+H]⁺ m/z: calcd 307.13; found 308.2; Rt=1.100 min.

### Step 2

To a solution of tert-butyl 3-(1,3-thiazol-4-yl)-1H,4H,5H,6H,7H-pyrazolo[4,3-c]pyridine-5-carboxylate (179.97 mg, 587.41 μmol) in dry DCM (3 mL) was added 4M HCl in dioxane (1.5 mL). The reaction mixture was stirred at r.t. overnight. The mixture was concentrated, the residue was dried under vacuum to afford 4-1H,4H,5H,6H,7H-pyrazolo[4,3-c]pyridin-3-yl-1,3-thiazole dihydrochloride (140.0 mg, 501.45 μmol, 85.4% yield) as a yellow solid.

LCMS(ESI): [M+H]⁺ m/z: calcd 207.07; found 207.0; Rt=0.381 min.

### Step 3

To a solution of [(dimethylamino)(3H-[1,2,3]triazolo[4,5-b]pyridin-3-yloxy)methylidene]dimethylazanium; hexafluoro-lambda5-phosphanuide (190.44 mg, 500.84 μmol) in DMF (0.5 mL) was added indolizine-2-carboxylic acid (80.71 mg, 500.84 μmol). The mixture was stirred for 10 mins, then 4-1H,4H,5H,6H,7H-pyrazolo[4,3-c]pyridin-3-yl-1,3-thiazole dihydrochloride (139.83 mg, 500.84 μmol) and triethylamine (253.4 mg, 2.5 mmol) were added. The reaction mixture was stirred at r.t. overnight. The product was purified directly by HPLC to afford 2-[3-(1,3-thiazol-4-yl)-1H,4H,5H,6H,7H-pyrazolo[4,3-c]pyridine-5-carbonyl]indolizine (45.5 mg, 130.22 μmol, 26% yield) as a yellow solid 1H NMR (400 MHz, DMSO) δ 12.99 (m, 1H), 9.25 (m, 1H), 8.26 (d, J=6.9 Hz, 1H), 7.87 (m, 2H), 7.44 (d, J=9.0 Hz, 1H), 6.75 (m, 1H), 6.62 (m, 2H), 4.88 (m, 2H), 3.92 (m, 2H), 2.84 (m, 2H).

LCMS (ESI): [M+H]⁺ m/z: calcd 350.1; found 350.2; Rt=2.604 min.

### Example 38

### 2-[3-(2,2-difluoromorpholine-4-carbonyl)-2H,4H,5H,6H,7H-pyrazolo[4,3-c]pyridine-5-carbonyl]indolizine

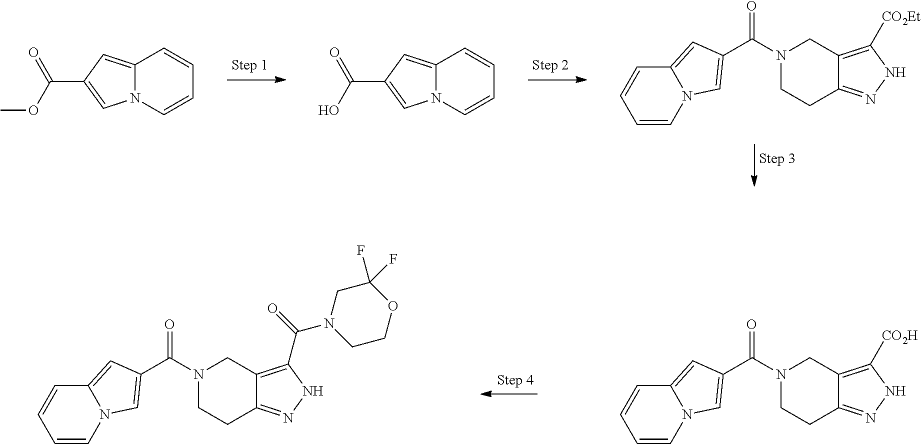 [SMILES: COC(=O)c1cc2ccccn2c1 ; O=C(O)c1cc2ccccn2c1 ; CCOC(=O)c1[nH]nc2c1CN(C(=O)c1cc3ccccn3c1)CC2 ; O=C(c1cc2ccccn2c1)N1CCc2n[nH]c(C(=O)N3CCOC(F)(F)C3)c2C1 ; O=C(O)c1[nH]nc2c1CN(C(=O)c1cc3ccccn3c1)CC2]

### Step 1

To a solution of methyl indolizine-2-carboxylate (6.5 g, 37.1 mmol) in a mixture of MeOH/THF/H₂O (4/4/1) (10 mL) was added lithium hydroxide monohydrate (3.11 g, 74.21 mmol). After stirring at 50° C. overnight, the reaction mixture was concentrated under reduced pressure. The obtained solution was cooled to 0-5° C. and acidified to pH 3-4 with 1M HCl. The suspension was stirred for 30 min and filtered. The filter cake was dried to dryness to afford indolizine-2-carboxylic acid (4.5 g, 27.92 mmol, 75.3% yield).

### Step 2

To a stirred solution of indolizine-2-carboxylic acid (2.17 g, 13.44 mmol) and [(dimethylamino)(3H-[1,2,3]triazolo[4,5-b]pyridin-3-yloxy)methylidene]dimethylazanium; hexafluoro-lambda5-phosphanuide (6.64 g, 17.47 mmol) in DMF (2 mL) was added ethyl 1H,4H,5H,6H,7H-pyrazolo[4,3-c]pyridine-3-carboxylate dihydrochloride (3.6 g, 13.44 mmol) and DIPEA (6.08 g, 47.02 mmol, 8.19 mL, 3.5 equiv.). After stirring overnight at RT, the reaction mixture was poured into water and extracted with EtOAc (2×15 mL). The combined organic fractions were washed three times with water, dried over anhydrous sodium sulfate and concentrated under reduced pressure. The crude product was purified by HPLC to give ethyl 5-(indolizine-2-carbonyl)-2H,4H,5H,6H,7H-pyrazolo[4,3-c]pyridine-3-carboxylate (900.0 mg, 2.66 mmol, 19.8% yield).

LCMS (ESI): [M+H]⁺ m/z: calcd. 339.15; found 339.2; Rt=0.952 min.

### Step 3

To a solution of ethyl 5-(indolizine-2-carbonyl)-2H,4H,5H,6H,7H-pyrazolo[4,3-c]pyridine-3-carboxylate (900.0 mg, 2.66 mmol) in a mixture of MeOH/THF/H₂O (4/4/1) (10 mL) was added lithium hydroxide monohydrate (278.94 mg, 6.65 mmol). After stirring at 50° C. overnight, the reaction mixture was concentrated under reduced pressure. The obtained solution was cooled to 0-5° C. and acidified to pH 3-4 with 1M HCl. The suspension was stirred for 30 min and filtered. The filter cake was dried to dryness to afford 5-(indolizine-2-carbonyl)-2H,4H,5H,6H,7H-pyrazolo[4,3-c]pyridine-3-carboxylic acid (800.0 mg, 97.0% purity, 2.5 mmol, 84.2% yield).

LCMS (ESI): [M+H]⁺ m/z: calcd 311.11; found 311.0; Rt=0.962 min.

### Step 4

To a stirred solution of 5-(indolizine-2-carbonyl)-2H,4H,5H,6H,7H-pyrazolo[4,3-c]pyridine-3-carboxylic acid (1 equiv.) and [(dimethylamino)(3H-[1,2,3[triazolo[4,5-b]pyridin-3-yloxy)methylidene]dimethylazanium; hexafluoro-lambda5-phosphanuide (1.2 equiv.) in DMF were added 2,2-difluoromorpholine hydrochloride (1 equiv.) and triethylamine (2.5 equiv.). After stirring overnight at RT the reaction mixture was poured into water and extracted with EtOAc. The combined organic fractions were washed three times with water, dried over anhydrous sodium sulfate and concentrated under reduced pressure. The crude product was purified by HPLC to give the desired product.

LCMS (ESI): [M+H]⁺ m/z: calcd. 416.16; found 416.2; Rt=3.181 min.

1H NMR (400 MHz, DMSO) δ 13.23 (s, 1H), 8.26 (d, J=6.9 Hz, 1H), 7.84 (s, 1H), 7.45 (d, J=9.0 Hz, 1H), 6.76 (m, 1H), 6.62 (t, J=6.6, 6.6 Hz, 1H), 6.58 (s, 1H), 4.77 (m, 3H), 4.35 (m, 1H), 4.11 (m, 2H), 4.00 (m, 1H), 3.88 (t, J=5.5, 5.5 Hz, 2H), 3.74 (m, 1H), 2.84 (m, 2H).

### Example 39

### 2-{3-[2-(trifluoromethyl)morpholine-4-carbonyl]-2H,4H,5H,6H,7H-pyrazolo[4,3-c]pyridine-5-carbonyl}indolizine

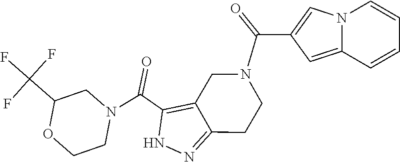 [SMILES: O=C(c1cc2ccccn2c1)N1CCc2n[nH]c(C(=O)N3CCOC(C(F)(F)F)C3)c2C1]

Prepared in an analogous fashion to Example 38.

LCMS (ESI): [M+H]⁺ m/z: calcd 448.17; found 448.2; Rt=2.734 min.

1H NMR (400 MHz, DMSO) δ 13.16 (m, 1H), 8.26 (d, J=7.0 Hz, 1H), 7.84 (s, 1H), 7.44 (d, J=9.0 Hz, 1H), 6.75 (m, 1H), 6.61 (t, J=6.7, 6.7 Hz, 1H), 6.58 (s, 1H), 5.24 (m, 1H), 4.86 (m, 2H), 4.37 (m, 2H), 3.99 (m, 1H), 3.87 (m, 2H), 3.62 (m, 1H), 3.45 (m, 1H), 3.00 (m, 1H), 2.82 (m, 2H).

### Example 40

### 5-(indolizine-2-carbonyl)-N-[(oxan-4-yl)methyl]-2H,4H,5H,6H,7H-pyrazolo[4,3-c]pyridine-3-carboxamide

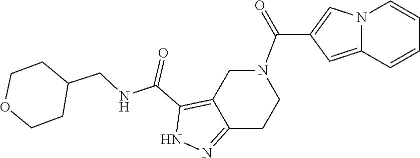 [SMILES: O=C(NCC1CCOCC1)c1[nH]nc2c1CN(C(=O)c1cc3ccccn3c1)CC2]

Prepared in an analogous fashion to Example 38.

LCMS (ESI): [M+H]⁺ m/z: calcd 408.22; found 408.2; Rt=2.245 min.

1H NMR (400 MHz, DMSO) δ 13.03 (s, 1H), 8.26 (d, J=6.6 Hz, 1H), 8.09 (m, 1H), 7.84 (s, 1H), 7.44 (d, J=8.9 Hz, 1H), 6.76 (m, 1H), 6.62 (t, J=6.3, 6.3 Hz, 1H), 6.57 (s, 1H), 4.85 (m, 2H), 3.86 (t, J=5.2, 5.2 Hz, 2H), 3.79 (m, 2H), 3.22 (t, J=11.4, 11.4 Hz, 2H), 3.07 (m, 2H), 2.81 (m, 2H), 1.73 (m, 1H), 1.50 (m, 2H), 1.14 (m, 2H).

### Example 41

### 5-(indolizine-2-carbonyl)-N-[1-(oxetan-3-yl)ethyl]-2H,4H,5H,6H,7H-pyrazolo[4,3-c]pyridine-3-carboxamide

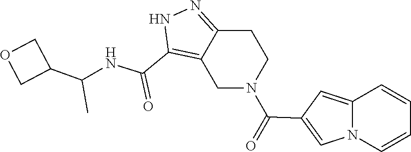 [SMILES: CC(NC(=O)c1[nH]nc2c1CN(C(=O)c1cc3ccccn3c1)CC2)C1COC1]

Prepared in an analogous fashion to Example 38.

LCMS (ESI): [M+H]⁺ m/z: calcd 394.2; found 394.2; Rt=2.120 min.

1H NMR (400 MHz, DMSO) δ 13.05 (s, 1H), 8.27 (d, J=7.5 Hz, 1H), 7.95 (d, J=9.2 Hz, 1H), 7.84 (s, 1H), 7.45 (d, J=8.7 Hz, 1H), 6.75 (m, 1H), 6.62 (m, 1H), 6.58 (s, 1H), 4.57 (m, 1H), 4.29 (m, 3H), 3.88 (m, 3H), 3.07 (m, 2H), 2.81 (m, 3H), 1.03 (m, 3H).

### Example 42

### 5-(indolizine-2-carbonyl)-N-[(3-methyloxetan-3-yl)methyl]-2H,4H,5H,6H,7H-pyrazolo[4,3-c]pyridine-3-carboxamide

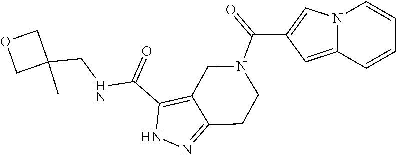 [SMILES: CC1(CNC(=O)c2[nH]nc3c2CN(C(=O)c2cc4ccccn4c2)CC3)COC1]

Prepared in an analogous fashion to Example 38.

LCMS (ESI): [M+H]⁺ m/z: calcd 394.2; found 394.2; Rt=1.041 min.

### Example 43

### 2-[3-(1,3-thiazol-4-yl)-4H,5H,6H,7H-pyrazolo[1,5-a]pyrazine-5-carbonyl]indolizine

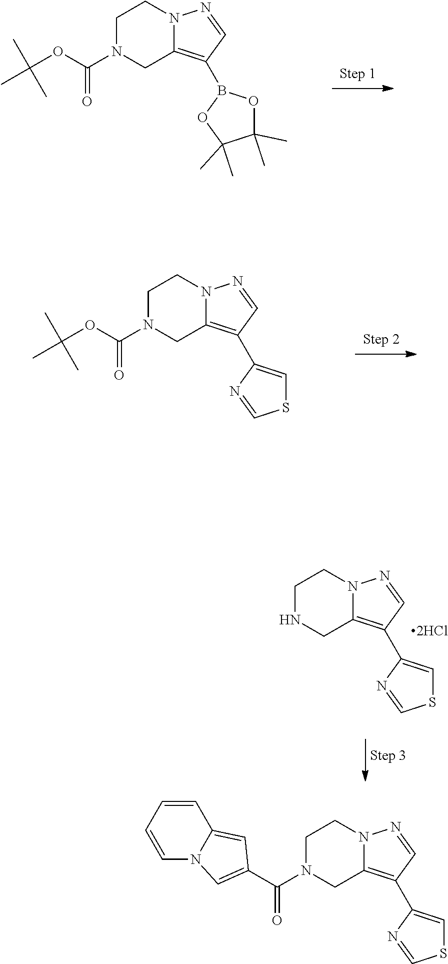

### Step 1

To a stirred suspension of 4-bromothiazole (774.98 mg, 4.72 mmol) in dioxane (70 mL) and H₂O (7 mL) were added tert-butyl 3-(tetramethyl-1,3,2-dioxaborolan-2-yl)-4H,5H,6H,7H-pyrazolo[1,5-a]pyrazine-5-carboxylate (1.5 g, 4.3 mmol) and cesium carbonate (2.38 g, 7.3 mmol). The reaction mixture was evacuated and backfilled with argon. [1,1′-Bis(diphenylphosphino)ferrocene]dichloropalladium(II), complex with dichloromethane (175.38 mg, 214.7 μmol) was added. After stirring at 98° C. (the temperature of oil bath) for 18 h (argon atmosphere), the reaction mixture was cooled, filtered through a pad of SiO₂ (30 g) and washed with dioxane (200 mL). The filtrate was concentrated in vacuo to give a crude product, which was purified by column chromatography (MTBE-hexane 4:1, Rf=0.3) to give tert-butyl thiazol-4-yl)-4H,5H,6H,7H-pyrazolo[1,5-a]pyrazine-5-carboxylate (700.0 mg, 2.06 mmol, 48% yield).

LCMS (ESI): [1\4+H]+m/z: calcd 307.13; found 307.0; Rt=1.168 min.

¹H NMR (500 MHz, CDCl₃) δ 1.483 (s, 9H), 3.916 (m, 2H), 4.211 (m, 2H), 4.932 (s, 2H), 7.144 (s, 1H), 7.848 (s, 1H), 8.793 (s, 1H).

### Step 2

To a solution of tert-butyl 3-(1,3-thiazol-4-yl)-4H,5H,6H,7H-pyrazolo[1,5-a]pyrazine-5-carboxylate (700.0 mg, 2.28 mmol) in MeOH (40 mL) was added HCl in dioxane (15 mL, 4M solution) at r.t. and the resulting mixture was stirred overnight. The obtained precipitate was collected by filtration and washed with Et₂O (20 mL) to give 4-4H,5H,6H,7H-pyrazolo[1,5-a]pyrazin-3-yl-1,3-thiazole dihydrochloride (400.0 mg, 95.0% purity, 1.36 mmol, 59.6% yield) as a white solid.

LCMS (ESI): [M+H]⁺ m/z: calcd 207.07; found 207.0; Rt=0.456 min.

¹H NMR (500 MHz, DMSO-d6) δ 4.390 (m, 3H), 4.620 (m, 3H), 7.837 (s, 1H), 8.033 (s, 1H), 9.183 (s, 1H), 10.296 (bds, 1H).

### Step 3

A solution of 4-4H,5H,6H,7H-pyrazolo[1,5-a]pyrazin-3-yl-1,3-thiazole dihydrochloride (147.82 mg, 529.46 μmol), indolizine-2-carboxylic acid (85.33 mg, 529.46 μmol) and [(dimethylamino)(3H-[1,2,3]triazolo [4,5-b]pyridin-3-yloxy)methylidene]dimethylazanium; hexafluoro-lambda5-phosphanuide (261.71 mg, 688.3 μmol) was stirred in DMF (2 mL) for 5 min at RT. DIPEA (341.32 mg, 2.64 mmol, 460 μL) was added in one portion. After stirring overnight (18 h) at RT, the reaction mixture was poured into water (30 mL) and extracted with EtOAc (3×30 mL). The combined organic fractions were washed with H₂O (2×30 mL), dried over anhydrous Na₂SO₄, and concentrated in vacuo to give a crude product (200 mg, 90% purity) which was purified by HPLC (2-10 min 50-70% water-MeOH; flow: 30 mL/min, column Waters SunFire C18, 100×19 mm, 5 μm) to give 2-[3-(1,3-thiazol-4-yl)-4H,5H,6H,7H-pyrazolo[1,5-a]pyrazine-5-carbonyl]indolizine (65.0 mg, 98.0% purity, 182.31 μmol, 34.4% yield) as a white solid.

LCMS (ESI): [M+H]⁺ m/z: calcd 350.11; found 350.0; Rt=1.013 min.

1H NMR (400 MHz, DMSO) δ 9.17 (s, 1H), 8.27 (d, J=6.5 Hz, 1H), 7.98 (s, 1H), 7.93 (s, 1H), 7.74 (s, 1H), 7.45 (d, J=8.9 Hz, 1H), 6.77 (t, J=7.5, 7.5 Hz, 1H), 6.64 (m, 2H), 5.19 (m, 2H), 4.29 (m, 2H), 4.18 (m, 2H).

### Example 44

### 6,8-difluoro-2-[6-methyl-3-(1,3-thiazol-4-yl)-1H,4H,5H,6H,7H-pyrazolo[4,3-c]pyridine-5-carbonyl]indolizine

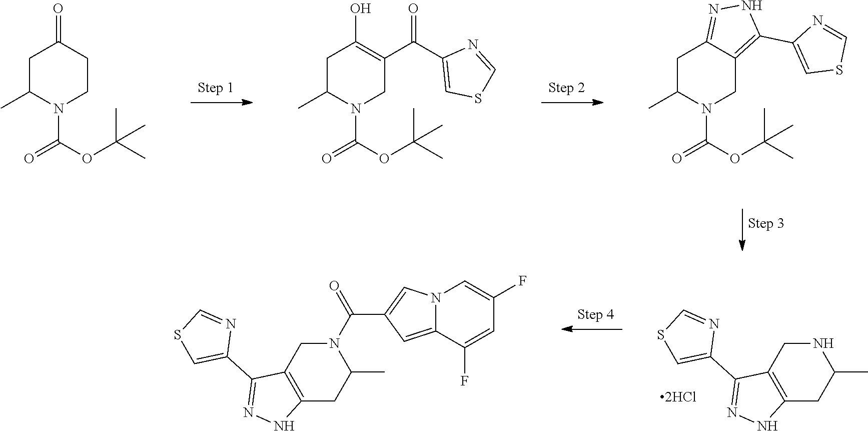 [SMILES: CC1CC(=O)CCN1C(=O)OC(C)(C)C ; CC1CC(O)=C(C(=O)c2cscn2)CN1C(=O)OC(C)(C)C ; CC1Cc2n[nH]c(-c3cscn3)c2CN1C(=O)OC(C)(C)C ; CC1Cc2[nH]nc(-c3cscn3)c2CN1C(=O)c1cc2c(F)cc(F)cn2c1 ; CC1Cc2[nH]nc(-c3cscn3)c2CN1]

### Step 1

Lithium hexamethyldisilazide (669.3 mg, 4.0 mmol, 4.0 mL, 2.0 equiv.) was added dropwise to a stirred solution of tert-butyl 2-methyl-4-oxopiperidine-1-carboxylate (640.03 mg, 3.0 mmol) in THF (5 mL) at −78° C. under an atmosphere of argon. The reaction was stirred at −78° C. for 10 min and then 1,3-thiazole-4-carbonyl chloride (295.26 mg, 2.0 mmol) in THF (2 mL) was added dropwise. The reaction was stirred at r.t. for 2 h, then acidified with 1M aqueous hydrochloric acid to pH 1. The resulting mixture was extracted with ethyl acetate (3×100 mL). The combined organic extracts were dried over Na₂SO₄, filtered and concentrated to afford crude tert-butyl 4-hydroxy-2-methyl-5-(1,3-thiazole-4-carbonyl)-1,2,3,6-tetrahydropyridine-1-carboxylate (700.0 mg, 80.0% purity, 1.73 mmol, 86.3% yield) as a brown oil that was used in the next step without further purification.

LCMS (ESI): [M-boc]⁺ m/z: calcd 225.13; found 225.0; Rt=1.487 min.

### Step 2

To a solution tert-butyl 4-hydroxy-2-methyl-5-(1,3-thiazole-4-carbonyl)-1,2,3,6-tetrahydropyridine-1-carboxylate (700.0 mg, 2.16 mmol) in EtOH (2 mL) were added hydrazine hydrate (215.92 mg, 4.31 mmol, 220.0 μL, 1.2 equiv) and acetic acid (194.26 mg, 3.23 mmol, 190.0 μL, 1.5 equiv). The reaction mixture was stirred at 70° C. for 5 h, then concentrated, the residue was dissolved in EtOAc (50 mL). The solution was washed with sat. aq. NaHCO₃, water, dried over Na₂SO₄ and concentrated. The residue was purified by HPLC to afford tert-butyl 6-methyl-3-(1,3-thiazol-4-yl)-1H,4H,5H,6H,7H-pyrazolo[4,3-c]pyridine-5-carboxylate (180.0 mg, 561.78 μmol, 26% yield) as yellow solid as a mixture of regioisomers.

LCMS (ESI): [M+H]⁺ m/z: calcd 321.15; found 321.2; Rt=2.936 min.

### Step 3

To a solution of tert-butyl 6-methyl-3-(1,3-thiazol-4-yl)-1H,4H,5H,6H,7H-pyrazolo[4,3-c]pyridine-5-carboxylate (180.3 mg, 562.73 μmol) in MeOH (1 mL) was added HCl in dioxane (4M, 2 mL). The reaction mixture was stirred at r.t. overnight, then concentrated. The residue was dried to afford 4-6-methyl-1H,4H,5H,6H,7H-pyrazolo[4,3-c]pyridin-3-yl-1,3-thiazole dihydrochloride (150.0 mg, 511.57 μmol, 90.9% yield) as a beige solid.

### Step 4

To a solution of 6,8-difluoroindolizine-2-carboxylic acid (101.67 mg, 515.75 μmol) in DMF (0.5 mL) was added [(dimethylamino)(3H-[1,2,3]triazolo[4,5-b]pyridin-3-yloxy)methylidene]dimethylazanium; hexafluoro-lambda5-phosphanuide (215.71 mg, 567.33 μmol). The mixture was stirred for 10 mins, then 4-6-methyl-1H,4H,5H,6H,7H-pyrazolo[4,3-c]pyridin-3-yl-1,3-thiazole dihydrochloride (151.23 mg, 515.75 μmol) and triethylamine (261.36 mg, 2.58 mmol, 360.0 μL, 5.0 equiv) were added. The reaction mixture was stirred at r.t. overnight. The product was purified directly by HPLC to afford 6,8-difluoro-2-[6-methyl-3-(1,3-thiazol-4-yl)-1H,4H,5H,6H,7H-pyrazolo[4,3-c]pyridine-5-carbonyl]indolizine (23.9 mg, 59.84 μmol, 11.6% yield) as a yellow solid.

LCMS (ESI): [M+H]⁺ m/z: calcd 400.11; found 400.2; Rt=3.087 min.

1H NMR (400 MHz, CDCl₃) δ 8.91 (s, 1H), 7.71 (m, 1H), 7.67 (s, 1H), 7.52 (s, 1H), 6.76 (s, 1H), 6.44 (t, J=9.4, 9.4 Hz, 1H), 5.54 (m, 1H), 5.01 (m, 1H), 4.37 (m, 1H), 3.14 (dd, J=15.5, 5.8 Hz, 1H), 2.73 (d, J=15.9 Hz, 1H), 1.26 (d, J=6.3 Hz, 3H).

### Example 45

### 5-(indolizine-2-carbonyl)-N-[(2S)-1,1,1-trifluoropropan-2-yl]-2H,4H,5H,6H,7H-pyrazolo[4,3-c]pyridine-3-carboxamide

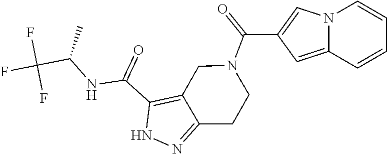 [SMILES: C[C@H](NC(=O)c1[nH]nc2c1CN(C(=O)c1cc3ccccn3c1)CC2)C(F)(F)F]

LCMS (ESI): [M+H]⁺ m/z: calcd. 406.16; found 406.2; Rt=2.977 min.

### Example 46

### 5-(indolizine-2-carbonyl)-N-[(2R)-1,1,1-trifluoropropan-2-yl]-2H,4H,5H,6H,7H-pyrazolo[4,3-c]pyridine-3-carboxamide

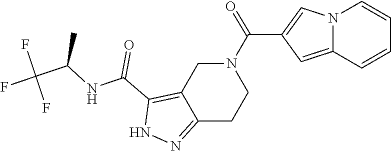 [SMILES: C[C@@H](NC(=O)c1[nH]nc2c1CN(C(=O)c1cc3ccccn3c1)CC2)C(F)(F)F]

LCMS (ESI): [M+H]⁺ m/z: calcd. 406.16; found 406.2; Rt=2.977 min.

### Example 47

### 5-(8-chloroindolizine-2-carbonyl)-6-methyl-N-(1,1,1-trifluoropropan-2-yl)-2H,4H,5H,6H,7H-pyrazolo[4,3-c]pyridine-3-carboxamide

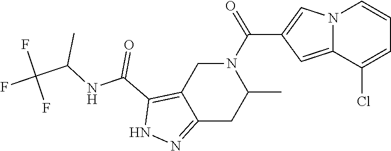 [SMILES: CC1Cc2n[nH]c(C(=O)NC(C)C(F)(F)F)c2CN1C(=O)c1cc2c(Cl)cccn2c1]

LCMS (ESI): [M+H]⁺ m/z: calcd. 454.12; found 454.2; Rt=3.471 min.

### Example 48

### 5-(indolizine-2-carbonyl)-6-methyl-N-(1,1,1-trifluoropropan-2-yl)-2H,4H,5H,6H,7H-pyrazolo[4,3-c]pyridine-3-carboxamide

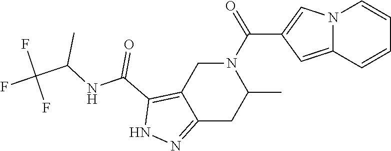 [SMILES: CC1Cc2n[nH]c(C(=O)NC(C)C(F)(F)F)c2CN1C(=O)c1cc2ccccn2c1]

LCMS (ESI): [M+H]⁺ m/z: calcd. 419.16; found 420.2; Rt=3.198 min.

### Example 49

### 5-(8-chloroindolizine-2-carbonyl)-N-(1,1,1-trifluoropropan-2-yl)-2H,4H,5H,6H,7H-pyrazolo[4,3-c]pyridine-3-carboxamide

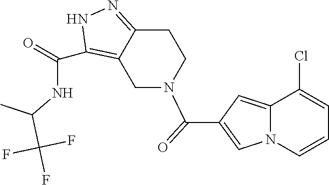 [SMILES: CC(NC(=O)c1[nH]nc2c1CN(C(=O)c1cc3c(Cl)cccn3c1)CC2)C(F)(F)F]

### Example 50

### 5-(indolizine-2-carbonyl)-N-(1,1,1-trifluoropropan-2-yl)-4H,5H,6H,7H-pyrazolo[1,5-a]pyrazine-3-carboxamide

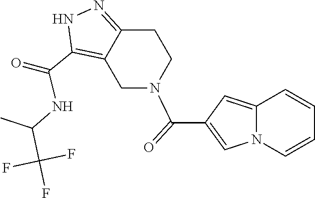 [SMILES: CC(NC(=O)c1[nH]nc2c1CN(C(=O)c1cc3ccccn3c1)CC2)C(F)(F)F]

### Step 1

5-[(Tert-butoxy)carbonyl]-4H,5H,6H,7H-pyrazolo[1,5-a]pyrazine-3-carboxylic acid (150.0 mg, 561.21 μmol) and [(dimethylamino)(3H-[1,2,3]triazolo[4,5-b]pyridin-3-yloxy)methylidene]dimethylazanium; hexafluoro-lambda5-phosphanuide (213.02 mg, 560.24 μmol) were mixed in DCM (3 ml) and stirred for 10 min. Then 1,1,1-trifluoropropan-2-amine (82.36 mg, 728.31 μmol) and triethylamine (113.38 mg, 1.12 mmol) were added and stirring was continued at r.t. overnight. The reaction mixture was washed with water (3×3 ml). The organic phase was separated, dried over sodium sulphate, filtered and concentrated in vacuo to afford tert-butyl 3-[(1,1,1-trifluoropropan-2-yl)carbamoyl]-4H,5H,6H,7H-pyrazolo[1,5-a]pyrazine-5-carboxylate (170.0 mg, 76.0% purity, 356.56 μmol, 63.6% yield) as a yellow oil.

LCMS(ESI): [M+H]⁺ m/z: calcd 363.18; found 363.1; Rt=1.238 min.

### Step 2

To a solution of tert-butyl 3-[(1,1,1-trifluoropropan-2-yl)carbamoyl]-4H,5H,6H,7H-pyrazolo[1,5-a]pyrazine-5-carboxylate (169.84 mg, 468.71 μmol) in DCM (0.5 ml) dioxane/HCl (0.6 ml, 4M) was added. After stirring at r.t. overnight, the reaction mixture was concentrated in vacuo. The residue was triturated with EtOAc and filtered off to afford N-(1,1,1-trifluoropropan-2-yl)-4H,5H,6H,7H-pyrazolo[1,5-a[pyrazine-3-carboxamide hydrochloride (70.0 mg, 234.35 μmol, 50% yield) as a white solid

LCMS(ESI): [M+H]⁺ m/z: calcd 263.12; found 263.2; Rt=0.577 min.

### Step 3

[(Dimethylamino)(3H-[1,2,3]triazolo [4,5-b]pyridin-3-yloxy)methylidene]dimethylazanium; hexafluoro-lambda5-phosphanuide (88.64 mg, 233.12 μmol) and indolizine-2-carboxylic acid (37.57 mg, 233.12 μmol) were mixed in DMF (1 ml) and stirred for 10 min N-(1,1,1-trifluoropropan-2-yl)-4H,5H,6H,7H-pyrazolo[1,5-a]pyrazine-3-carboxamide hydrochloride (69.63 mg, 233.12 μmol) and triethylamine (94.38 mg, 932.7 μmol, 130.0 μl, 4.0 equiv) were added and stirring was continued at r.t. overnight. The crude reaction mixture was subjected to HPLC (30-80% 0-5 min water-MeOH, flow 30 ml/min (loading pump 4 ml/min MeOH), target mass 405.38 column: SunFireC18 100*19 mm Sum) to give 5-(indolizine-2-carbonyl)-N-(1,1,1-trifluoropropan-2-yl)-4H,5H,6H,7H-pyrazolo[1,5-a]pyrazine-3-carboxamide (27.2 mg, 67.1 μmol, 28.8% yield) as a brown solid.

LCMS (ESI): [M+H]⁺ m/z: calcd. 406.2; found 406.4; Rt=3.104 min.

1H NMR (DMSO-d6, 400 MHz): δ 8.42 (d, J=8.7 Hz, 1H), 8.25 (d, J=6.8 Hz, 1H), 8.10 (s, 1H), 7.88 (s, 1H), 7.43 (d, 1H), 6.75 (m, 1H), 6.61 (m, 2H), 5.08 (m, 2H), 4.77 (m, 1H), 4.19 (m, 3H), 4.04 (m, 1H), 1.28 (d, J=6.0 Hz, 3H)

### Example 51

### 5-(indolizine-2-carbonyl)-N-(1,1,1-trifluoropropan-2-yl)-4H,5H,6H,7H-[1,2]oxazolo[4,5-c]pyridine-3-carboxamide

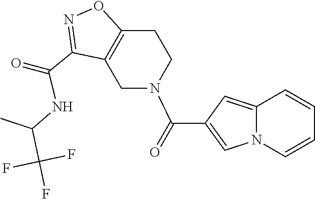 [SMILES: CC(NC(=O)c1noc2c1CN(C(=O)c1cc3ccccn3c1)CC2)C(F)(F)F]

LCMS (ESI): [M+H]⁺ m/z: calcd. 407.2; found 407.4; Rt=3.448 min.

### Example 52

### 5-(indolizine-2-carbonyl)-N-[1,1,1-trifluoropropan-2-yl]-2H,4H,5H,6H,7H-pyrazolo[4,3-c]pyridine-3-carboxamide

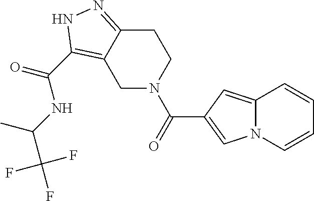 [SMILES: CC(NC(=O)c1[nH]nc2c1CN(C(=O)c1cc3ccccn3c1)CC2)C(F)(F)F]

LCMS (ESI): [M+H]⁺ m/z: calcd. 406.2; found 407.4; Rt=3.160 min.

### Example 53

### 5-(indolizine-2-carbonyl)-N-(1,1,1-trifluoropropan-2-yl)-4H,5H,6H,7H-[1,2]oxazolo[4,3-c]pyridine-3-carboxamide

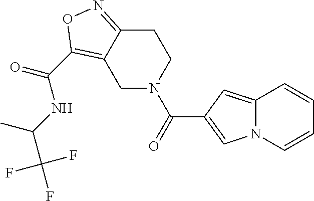 [SMILES: CC(NC(=O)c1onc2c1CN(C(=O)c1cc3ccccn3c1)CC2)C(F)(F)F]

LCMS (ESI): [M+H]⁺ m/z: calcd. 407.1; found 407.4; Rt=3.437 min.

Selected compounds of the invention were assayed in capsid assembly and HBV replication assays, as described below and a representative group of these active compounds is shown in Table 1 and Table 2.

Biochemical Capsid Assembly Assay

The screening for assembly effector activity was done based on a fluorescence quenching assay published by Zlotnick et al. (2007). The C-terminal truncated core protein containing 149 amino acids of the N-terminal assembly domain fused to a unique cysteine residue at position 150 and was expressed in E. coli using the pET expression system (Merck Chemicals, Darmstadt). Purification of core dimer protein was performed using a sequence of size exclusion chromatography steps. In brief, the cell pellet from 1 L BL21 (DE3) Rosetta2 culture expressing the coding sequence of core protein cloned NdeI/XhoI into expression plasmid pET21b was treated for 1 h on ice with a native lysis buffer (Qproteome Bacterial Protein Prep Kit; Qiagen, Hilden). After a centrifugation step the supernatant was precipitated during 2 h stirring on ice with 0.23 g/ml of solid ammonium sulfate. Following further centrifugation the resulting pellet was resolved in buffer A (100 mM Tris, pH 7.5; 100 mM NaCl; 2 mM DTT) and was subsequently loaded onto a buffer A equilibrated CaptoCore 700 column (GE HealthCare, Frankfurt). The column flow through containing the assembled HBV capsid was dialyzed against buffer N (50 mM NaHCO₃ pH 9.6; 5 mM DTT) before urea was added to a final concentration of 3M to dissociate the capsid into core dimers for 1.5 h on ice. The protein solution was then loaded onto a 1 L Sephacryl 5300 column. After elution with buffer N core dimer containing fractions were identified by SDS-PAGE and subsequently pooled and dialyzed against 50 mM HEPES pH 7.5; 5 mM DTT. To improve the assembly capacity of the purified core dimers a second round of assembly and disassembly starting with the addition of 5 M NaCl and including the size exclusion chromatography steps described above was performed. From the last chromatography step core dimer containing fractions were pooled and stored in aliquots at concentrations between 1.5 to 2.0 mg/ml at −80° C.

Immediately before labelling the core protein was reduced by adding freshly prepared DTT in a final concentration of 20 mM. After 40 mM incubation on ice storage buffer and DTT was removed using a Sephadex G-25 column (GE HealthCare, Frankfurt) and 50 mM HEPES, pH 7.5. For labelling 1.6 mg/ml core protein was incubated at 4° C. and darkness overnight with BODIPY-FL maleimide (Invitrogen, Karlsruhe) in a final concentration of 1 mM. After labelling the free dye was removed by an additional desalting step using a Sephadex G-25 column. Labelled core dimers were stored in aliquots at 4° C. In the dimeric state the fluorescence signal of the labelled core protein is high and is quenched during the assembly of the core dimers to high molecular capsid structures. The screening assay was performed in black 384 well microtiter plates in a total assay volume of 10 μl using 50 mM HEPES pH 7.5 and 1.0 to 2.0 μM labelled core protein. Each screening compound was added in 8 different concentrations using a 0.5 log-unit serial dilution starting at a final concentration of 100 μM, 31.6 μM or 10 μM, In any case the DMSO concentration over the entire microtiter plate was 0.5%. The assembly reaction was started by the injection of NaCl to a final concentration of 300 μM which induces the assembly process to approximately 25% of the maximal quenched signal. 6 min after starting the reaction the fluorescence signal was measured using a Clariostar plate reader (BMG Labtech, Ortenberg) with an excitation of 477 nm and an emission of 525 nm. As 100% and 0% assembly control HEPES buffer containing 2.5 M and 0 M NaCl was used. Experiments were performed thrice in triplicates. EC₅₀ values were calculated by non-linear regression analysis using the Graph Pad Prism 6 software (GraphPad Software, La Jolla, USA).

Determination of HBV DNA from the Supernatants of HepAD38 Cells

The anti-HBV activity was analysed in the stable transfected cell line HepAD38, which has been described to secrete high levels of HBV virion particles (Ladner et al., 1997). In brief, HepAD38 cells were cultured at 37° C. at 5% CO₂ and 95% humidity in 200 μl maintenance medium, which was Dulbecco's modified Eagle's medium/Nutrient Mixture F-12 (Gibco, Karlsruhe), 10% fetal bovine serum (PAN Biotech Aidenbach) supplemented with 50 μg/ml penicillin/streptomycin (Gibco, Karlsruhe), 2 mM L-glutamine (PAN Biotech, Aidenbach), 400 μg/ml G418 (AppliChem, Darmstadt) and 0.3 μg/ml tetracycline. Cells were subcultured once a week in a 1:5 ratio, but were usually not passaged more than ten times. For the assay 60,000 cells were seeded in maintenance medium without any tetracycline into each well of a 96-well plate and treated with serial half-log dilutions of test compound. To minimize edge effects the outer 36 wells of the plate were not used but were filled with assay medium. On each assay plate six wells for the virus control (untreated HepAD38 cells) and six wells for the cell control (HepAD38 cells treated with 0.3 μg/ml tetracycline) were allocated, respectively. In addition, one plate set with reference inhibitors like BAY 41-4109, entecavir, and lamivudine instead of screening compounds were prepared in each experiment. In general, experiments were performed thrice in triplicates. At day 6 HBV DNA from 100 μl filtrated cell culture supernatant (AcroPrep Advance 96 Filter Plate, 0.45 μM Supor membran, PALL GmbH, Dreieich) was automatically purified on the MagNa Pure LC instrument using the MagNA Pure 96 DNA and Viral NA Small Volume Kit (Roche Diagnostics, Mannheim) according to the instructions of the manufacturer. EC50 values were calculated from relative copy numbers of HBV DNA In brief, 5 μl of the 100 μl eluate containing HBV DNA were subjected to PCR LC480 Probes Master Kit (Roche) together with 1 μM antisense primer tgcagaggtgaagcgaagtgcaca, 0.5 μM sense primer gacgtcctttgtttacgtcccgtc, 0.3 μM hybprobes acggggcgcacctctctttacgcgg-FL and LC640-ctccccgtctgtgccttctcatctgc-PH (TIBMolBiol, Berlin) to a final volume of 12.5 pl. The PCR was performed on the Light Cycler 480 real time system (Roche Diagnostics, Mannheim) using the following protocol: Pre-incubation for 1 min at 95° C., amplification: 40 cycles×(10 sec at 95° C., 50 sec at 60° C., 1 sec at 70° C.), cooling for 10 sec at 40° C. Viral load was quantitated against known standards using HBV plasmid DNA of pCH-9/3091 (Nassal et al., 1990, Cell 63: 1357-1363) and the LightCycler 480 SW 1.5 software (Roche Diagnostics, Mannheim) and EC₅₀ values were calculated using non-linear regression with GraphPad Prism 6 (GraphPad Software Inc., La Jolla, USA).

Cell Viability Assay

Using the AlamarBlue viability assay cytotoxicity was evaluated in HepAD38 cells in the presence of 0.3 μg/ml tetracycline, which blocks the expression of the HBV genome. Assay condition and plate layout were in analogy to the anti-HBV assay, however other controls were used. On each assay plate six wells containing untreated HepAD38 cells were used as the 100% viability control, and six wells filled with assay medium only were used as 0% viability control. In addition, a geometric concentration series of cycloheximide starting at 60 μM final assay concentration was used as positive control in each experiment. After six days incubation period Alamar Blue Presto cell viability reagent (ThermoFisher, Dreieich) was added in 1/11 dilution to each well of the assay plate. After an incubation for 30 to 45 min at 37° C. the fluorescence signal, which is proportional to the number of living cells, was read using a Tecan Spectrafluor Plus plate reader with an excitation filter 550 nm and emission filter 595 nm, respectively. Data were normalized into percentages of the untreated control (100% viability) and assay medium (0% viability) before CC50 values were calculated using non-linear regression and the GraphPad Prism 6.0 (GraphPad Software, La Jolla, USA). Mean EC₅₀ and CC₅₀ values were used to calculate the selectivity index (SI=CC₅₀/EC₅₀) for each test compound.

In Vivo Efficacy Models

HBV research and preclinical testing of antiviral agents are limited by the narrow species- and tissue-tropism of the virus, the paucity of infection models available and the restrictions imposed by the use of chimpanzees, the only animals fully susceptible to HBV infection. Alternative animal models are based on the use of HBV-related hepadnaviruses and various antiviral compounds have been tested in woodchuck hepatitis virus (WHV) infected woodchucks or in duck hepatitis B virus (DHBV) infected ducks or in woolly monkey HBV (WM-HBV) infected tupaia (overview in Dandri et al., 2017, Best Pract Res Clin Gastroenterol 31, 273-279). However, the use of surrogate viruses has several limitations. For example is the sequence homology between the most distantly related DHBV and HBV is only about 40% and that is why core protein assembly modifiers of the HAP family appeared inactive on DHBV and WHV but efficiently suppressed HBV (Campagna et al., 2013, J. Virol. 87, 6931-6942). Mice are not HBV permissive but major efforts have focused on the development of mouse models of HBV replication and infection, such as the generation of mice transgenic for the human HBV (HBV tg mice), the hydrodynamic injection (HDI) of HBV genomes in mice or the generation of mice having humanized livers and/or humanized immune systems and the intravenous injection of viral vectors based on adenoviruses containing HBV genomes (Ad-HBV) or the adenoassociated virus (AAV-HBV) into immune competent mice (overview in Dandri et al., 2017, Best Pract Res Clin Gastroenterol 31, 273-279). Using mice transgenic for the full HBV genome the ability of murine hepatocytes to produce infectious HBV virions could be demonstrated (Guidotti et al., 1995, J. Virol., 69: 6158-6169). Since transgenic mice are immunological tolerant to viral proteins and no liver injury was observed in HBV-producing mice, these studies demonstrated that HBV itself is not cytopathic. HBV transgenic mice have been employed to test the efficacy of several anti-HBV agents like the polymerase inhibitors and core protein assembly modifiers (Weber et al., 2002, Antiviral Research 54 69-78; Julander et al., 2003, Antivir. Res., 59: 155-161), thus proving that HBV transgenic mice are well suitable for many type of preclinical antiviral testing in vivo.

As described in Paulsen et al., 2015, PLOSone, 10: e0144383 HBV-transgenic mice (Tg [HBV1.3 fsX⁻3′5′]) carrying a frameshift mutation (GC) at position 2916/2917 could be used to demonstrate antiviral activity of core protein assembly modifiers in vivo. In brief, The HBV-transgenic mice were checked for HBV-specific DNA in the serum by qPCR prior to the experiments (see section “Determination of HBV DNA from the supernatants of HepAD38 cells”). Each treatment group consisted of five male and five female animals approximately 10 weeks age with a titer of 10⁷-10⁸ virions per ml serum. Compounds were formulated as a suspension in a suitable vehicle such as 2% DMSO/98% tylose (0.5% Methylcellulose/99.5% PBS) or 50% PEG400 and administered per os to the animals one to three times/day for a 10 day period. The vehicle served as negative control, whereas 1 μg/kg entecavir in a suitable vehicle was the positive control. Blood was obtained by retro bulbar blood sampling using an Isoflurane Vaporizer. For collection of terminal heart puncture six hours after the last treatment blood or organs, mice were anaesthetized with isoflurane and subsequently sacrificed by CO₂ exposure. Retro bulbar (100-150 μl) and heart puncture (400-500 μl) blood samples were collected into a Microvette 300 LH or Microvette 500 LH, respectively, followed by separation of plasma via centrifugation (10 min, 2000 g, 4° C.). Liver tissue was taken and snap frozen in liquid N2. All samples were stored at −80° C. until further use. Viral DNA was extracted from 50 μl plasma or 25 mg liver tissue and eluted in 50 μl AE buffer (plasma) using the DNeasy 96 Blood & Tissue Kit (Qiagen, Hilden) or 320 μl AE buffer (liver tissue) using the DNeasy Tissue Kit (Qiagen, Hilden) according to the manufacturer's instructions. Eluted viral DNA was subjected to qPCR using the LightCycler 480 Probes Master PCR kit (Roche, Mannheim) according to the manufacturer's instructions to determine the HBV copy number. HBV specific primers used included the forward primer 5′-CTG TAC CAA ACC TTC GGA CGG-3′, the reverse primer 5′-AGG AGA AAC GGG CTG AGG C-3′ and the FAM labelled probe FAM-CCA TCA TCC TGG GCT TTC GGA AAA TT-BBQ. One PCR reaction sample with a total volume of 20 μl contained 5 μl DNA eluate and 15 μl master mix (comprising 0.3 μM of the forward primer, 0.3 μM of the reverse primer, 0.15 μM of the FAM labelled probe). qPCR was carried out on the Roche LightCycler1480 using the following protocol: Pre-incubation for 1 min at 95° C., amplification: (10 sec at 95° C., 50 sec at 60° C., 1 sec at 70° C.)×45 cycles, cooling for 10 sec at 40° C. Standard curves were generated as described above. All samples were tested in duplicate. The detection limit of the assay is 50 HBV DNA copies (using standards ranging from 250-2.5×107 copy numbers). Results are expressed as HBV DNA copies/10 μl plasma or HBV DNA copies/100 ng total liver DNA (normalized to negative control).

It has been shown in multiple studies that not only transgenic mice are a suitable model to proof the antiviral activity of new chemical entities in vivo the use of hydrodynamic injection of HBV genomes in mice as well as the use of immune deficient human liver chimeric mice infected with HBV positive patient serum have also frequently used to profile drugs targeting HBV (Li et al., 2016, Hepat. Mon. 16: e34420; Qiu et al., 2016, J. Med. Chem. 59: 7651-7666; Lutgehetmann et al., 2011, Gastroenterology, 140: 2074-2083). In addition chronic HBV infection has also been successfully established in immunecompetent mice by inoculating low doses of adenovirus-(Huang et al., 2012, Gastroenterology 142: 1447-1450) or adeno-associated virus (AAV) vectors containing the HBV genome (Dion et al., 2013, J Virol. 87: 5554-5563). This models could also be used to demonstrate the in vivo antiviral activity of novel anti-HBV agents.

TABLE 1
Capsid assembly assay
In Table 1, “A” represents an IC₅₀ <
5 μM; “B” represents 5 μM < IC₅₀ <
10 μM; “C” represents IC₅₀ < 100 μM

| Example | Assembly Activity |
|---|---|
| Example 3 | A |
| Example 4 | B |
| Example 5 | A |
| Example 6 | A |
| Example 7 | A |
| Example 8 | A |
| Example 9 | A |
| Example 10 | A |
| Example 12 | C |
| Example 16 | B |
| Example 17 | A |
| Example 18 | B |
| Example 19 | B |
| Example 20 | B |
| Example 21 | B |
| Example 22 | B |
| Example 23 | A |
| Example 24 | A |
| Example 25 | B |
| Example 26 | A |
| Example 28 | C |
| Example 29 | C |
| Example 30 | C |
| Example 31 | A |
| Example 33 | C |
| Example 34 | A |
| Example 35 | C |
| Example 36 | A |
| Example 37 | A |
| Example 41 | C |
| Example 43 | A |
| Example 44 | A |
| Example 45 | C |
| Example 46 | C |
| Example 47 | C |
| Example 48 | C |
| Example 51 | A |
| Example 53 | A |

TABLE 2
HBV Replication Assay
In Table 2, “+++” represents
an EC₅₀ < 1 μM; “++” represents 1 μM <
EC₅₀ < 10 μM; “+” represents EC₅₀ < 100 μM

| Example | Cell Activity |
|---|---|
| Example 1 | ++ |
| Example 2 | ++ |
| Example 3 | +++ |
| Example 4 | +++ |
| Example 5 | +++ |
| Example 6 | +++ |
| Example 7 | +++ |
| Example 8 | ++ |
| Example 9 | +++ |
| Example 10 | +++ |
| Example 11 | ++ |
| Example 12 | ++ |
| Example 16 | ++ |
| Example 17 | +++ |
| Example 18 | +++ |
| Example 19 | +++ |
| Example 20 | +++ |
| Example 21 | +++ |
| Example 22 | +++ |
| Example 23 | +++ |
| Example 24 | +++ |
| Example 25 | +++ |
| Example 26 | +++ |
| Example 49 | ++ |
| Example 50 | +++ |
| Example 51 | +++ |
| Example 52 | + |
| Example 53 | +++ |

1. Compound of Formula I 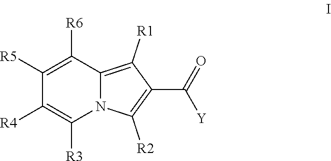
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 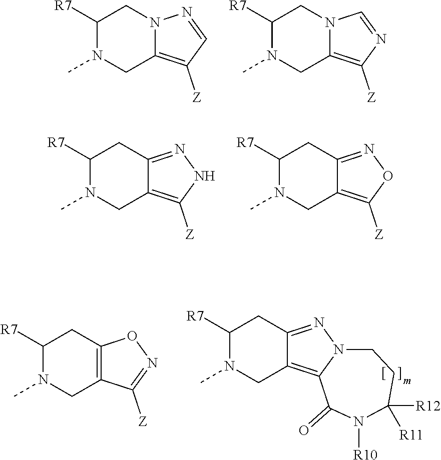
R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

2. A compound of Formula I according to claim 1 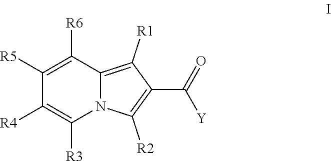
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 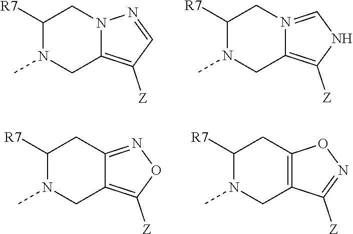
R7 is selected from the group comprising H, D, and C1-C4-alkyl
Z is selected from the group comprising C3-C6-cycloalkyl, C3-C7-heterocycloalkyl, thiazolyl, triazolyl, isoxazolyl and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, oxetanyl, tetrahydrofuranyl, tetrahydropyranyl, CH₂OH, CH₂OCH₃, CH₂CH₂OH, CF₃, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

3. A compound of Formula I according to claim 1 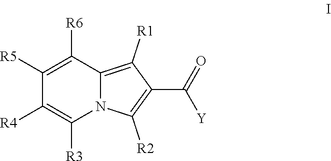
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 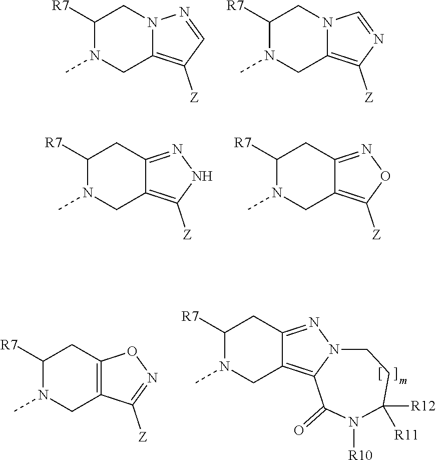
R7 is selected from the group comprising H, D, and C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

4. A compound of Formula I according to claim 1 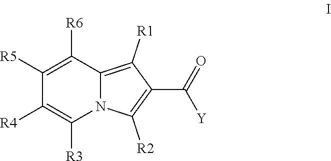
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
Y is selected from the group comprising 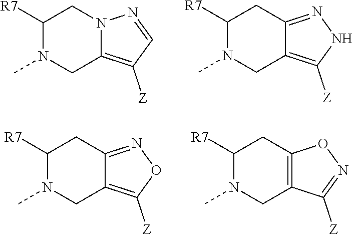
R7 is selected from the group comprising H, D, and C1-C4-alkyl
Z is selected from the group comprising C3-C6-cycloalkyl, C3-C7-heterocycloalkyl, thiazolyl, and C(═O)N(R^{a})(R^{b})
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
wherein the dashed line is a covalent bond between C(O) and Y, and
wherein heterocycloalkyl has 1 or 2 heteroatoms each independently selected from N, O and S,
or a pharmaceutically acceptable salt thereof or a solvate of a compound of Formula I or the pharmaceutically acceptable salt thereof or a prodrug of a compound of Formula I or a pharmaceutically acceptable salt or a solvate thereof.

5. A compound of Formula I according to claim 1, or a pharmaceutically acceptable salt thereof or a solvate of a compound of Formula I or the pharmaceutically acceptable salt thereof or a prodrug of a compound of Formula I or a pharmaceutically acceptable salt or a solvate thereof, wherein the prodrug is selected from the group consisting of esters and amides, preferably alkyl esters of fatty acids.

6. A compound of Formula I according to claim 1 that is a compound of Formula IIa 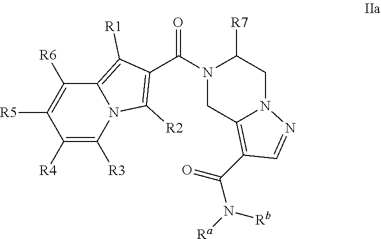
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano,
or a pharmaceutically acceptable salt thereof or a solvate of a compound of Formula Ha or the pharmaceutically acceptable salt thereof or a prodrug of a compound of Formula Ha or a pharmaceutically acceptable salt or a solvate thereof.

7. A compound of Formula I according to claim 1 that is a compound of Formula IIb 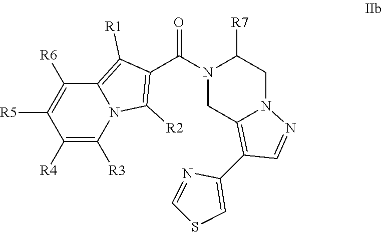
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl,
or a pharmaceutically acceptable salt thereof or a solvate of a compound of Formula IIb or the pharmaceutically acceptable salt thereof or a prodrug of a compound of Formula IIb or a pharmaceutically acceptable salt or a solvate thereof.

8. A compound of Formula I according to claim 1 that is a compound of Formula IIc 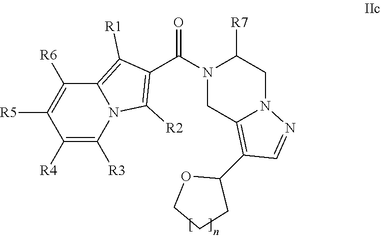
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3,
or a pharmaceutically acceptable salt thereof or a solvate of a compound of Formula IIc or the pharmaceutically acceptable salt thereof or a prodrug of a compound of Formula IIc or a pharmaceutically acceptable salt or a solvate thereof.

9. A compound of Formula I according to claim 1 that is a compound of Formula IIIa 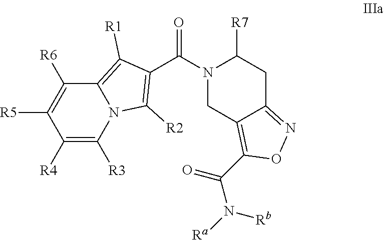
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano,
or a pharmaceutically acceptable salt thereof or a solvate of a compound of Formula Ina or the pharmaceutically acceptable salt thereof or a prodrug of a compound of Formula Ma or a pharmaceutically acceptable salt or a solvate thereof.

10. A compound of Formula I according to claim 1 that is a compound of Formula IIIb 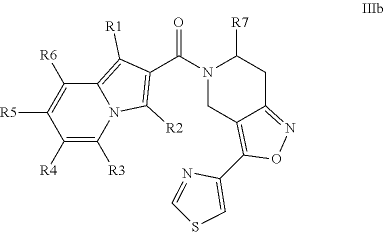
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl,
or a pharmaceutically acceptable salt thereof or a solvate of a compound of Formula IIIb or the pharmaceutically acceptable salt thereof or a prodrug of a compound of Formula IIIb or a pharmaceutically acceptable salt or a solvate thereof.

11. A compound of Formula I according to claim 1 that is a compound of Formula IIIc 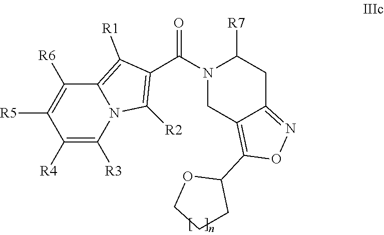
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3,
or a pharmaceutically acceptable salt thereof or a solvate of a compound of Formula IIIc or the pharmaceutically acceptable salt thereof or a prodrug of a compound of Formula IIIc or a pharmaceutically acceptable salt or a solvate thereof.

12. A compound of Formula I according to claim 1 that is a compound of Formula IVa 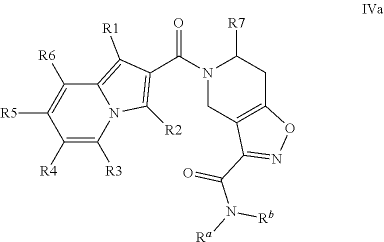
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano,
or a pharmaceutically acceptable salt thereof or a solvate of a compound of Formula IVa or the pharmaceutically acceptable salt thereof or a prodrug of a compound of Formula IVa or a pharmaceutically acceptable salt or a solvate thereof.

13. A compound of Formula I according to claim 1 that is a compound of Formula IVb 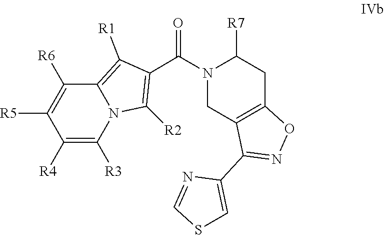
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl,
or a pharmaceutically acceptable salt thereof or a solvate of a compound of Formula IVb or the pharmaceutically acceptable salt thereof or a prodrug of a compound of Formula IVb or a pharmaceutically acceptable salt or a solvate thereof.

14. A compound of Formula I according to claim 1 that is a compound of Formula IVc 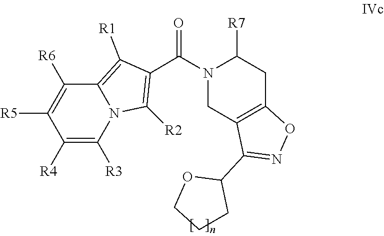
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3,
or a pharmaceutically acceptable salt thereof or a solvate of a compound of Formula IVc or the pharmaceutically acceptable salt thereof or a prodrug of a compound of Formula IVc or a pharmaceutically acceptable salt or a solvate thereof.

15. A compound of Formula I according to claim 1 that is a compound of Formula Va 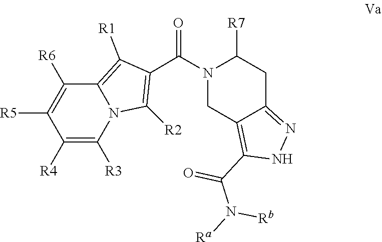
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano,
or a pharmaceutically acceptable salt thereof or a solvate of a compound of Formula Va or the pharmaceutically acceptable salt thereof or a prodrug of a compound of Formula Va or a pharmaceutically acceptable salt or a solvate thereof.

16. A compound of Formula I according to claim 1 that is a compound of Formula Vb 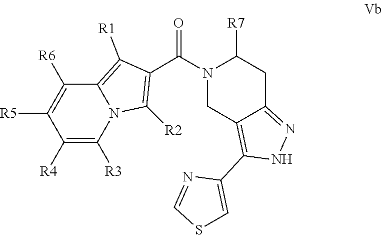
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl,
or a pharmaceutically acceptable salt thereof or a solvate of a compound of Formula Vb or the pharmaceutically acceptable salt thereof or a prodrug of a compound of Formula Vb or a pharmaceutically acceptable salt or a solvate thereof.

17. A compound of Formula I according to claim 1 that is a compound of Formula Vc 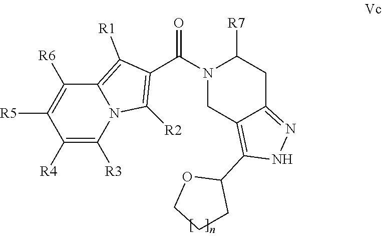
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3,
or a pharmaceutically acceptable salt thereof or a solvate of a compound of Formula Vc or the pharmaceutically acceptable salt thereof or a prodrug of a compound of Formula Vc or a pharmaceutically acceptable salt or a solvate thereof.

18. A compound of Formula I according to claim 1 that is a compound of Formula VIa 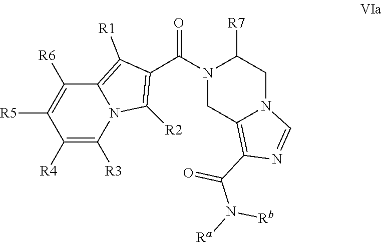
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R^{a} is selected from the group comprising H, methyl, ethyl, 2,2-difluoroethyl, 2,2,2-trifluoroethyl, 2-hydroxyethyl, and cyclopropyl
R^{b} is selected from the group comprising H, C1-C4-alkyl, C1-C4-haloalkyl, and C3-C6-cycloalkyl, optionally substituted with phenyl, pyridyl, pyrimidinyl, pyrazinyl, pyridazinyl, triazinyl, oxazolyl, isoxazolyl, imidazolyl, pyrazolyl, CH₂OH, CH₂OCH₃, CH₂CH₂OH, and CH₂OCHF₂, wherein phenyl, pyridyl, pyrimidinyl, pyrazinyl, pyridazinyl, triazinyl, oxazolyl, isoxazolyl, imidazolyl, and pyrazolyl are optionally substituted with 1, 2 or 3 groups each independently selected from C1-C4-alkyl, carboxy and halo
R^{a} and R^{b} are optionally connected to form a C3-C7-heterocycloalkyl ring or hetero-spirocyclic ring system consisting of 2 or 3 C3-C7 rings, optionally substituted with 1, 2, or 3 groups selected from OH, halogen, carboxy and cyano,
or a pharmaceutically acceptable salt thereof or a solvate of a compound of Formula VIa or the pharmaceutically acceptable salt thereof or a prodrug of a compound of Formula VIa or a pharmaceutically acceptable salt or a solvate thereof.

19. A compound of Formula I according to claim 1 that is a compound of Formula VIb 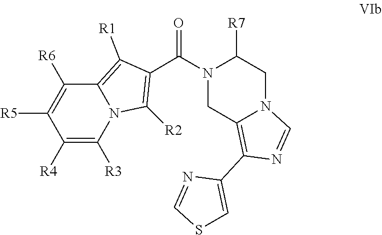
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl,
or a pharmaceutically acceptable salt thereof or a solvate of a compound of Formula VIb or the pharmaceutically acceptable salt thereof or a prodrug of a compound of Formula VIb or a pharmaceutically acceptable salt or a solvate thereof.

20. A compound of Formula I according to claim 1 that is a compound of Formula VIc 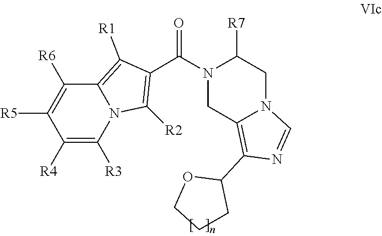
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
n is 0, 1, 2 or 3,
or a pharmaceutically acceptable salt thereof or a solvate of a compound of Formula VIc or the pharmaceutically acceptable salt thereof or a prodrug of a compound of Formula VIc or a pharmaceutically acceptable salt or a solvate thereof.

21. A compound of Formula I according to claim 1 that is a compound of Formula VII 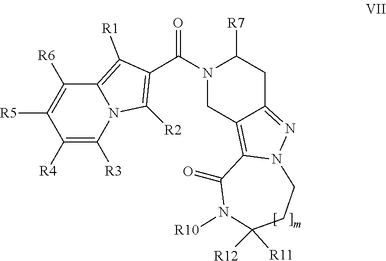
in which
R1, R2, R3, R4, R5, and R6, are for each position independently selected from the group comprising H, F, Cl, Br, I, CF₃, CF₂H, C1-C4-alkyl, CF₂CH₃, cyclopropyl, cyano, and nitro
R7 is selected from the group comprising H, D and C1-C4-alkyl
R10 is selected from the group comprising H, methyl, ethyl, 2,2-difluoroethyl, 2,2,2-trifluoroethyl, 2-hydroxyethyl, and cyclopropyl
R11 and R12 are independently selected from the group comprising H, methyl, and ethyl
R11 and R12 are optionally connected to form a C3-C5-cycloalkyl ring
m is 0, 1, 2 or 3,
or a pharmaceutically acceptable salt thereof or a solvate of a compound of Formula VII or the pharmaceutically acceptable salt thereof or a prodrug of a compound of Formula VII or a pharmaceutically acceptable salt or a solvate thereof.

22. A method for the prevention or treatment of an HBV infection in a subject, comprising administering to the subject a therapeutically effective amount of a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.

23. A pharmaceutical composition comprising a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof, together with a pharmaceutically acceptable carrier.

24. A method of treating an HBV infection in an individual in need thereof, comprising administering to the individual a therapeutically effective amount of a compound according to claim 1 or a pharmaceutically acceptable salt thereof or a solvate or a hydrate of said compound or the pharmaceutically acceptable salt thereof or a prodrug of said compound or a pharmaceutically acceptable salt or a solvate or a hydrate thereof.

25. Method for the preparation of a compound of Formula I as defined in claim 1 by reacting a compound of Formula VIII 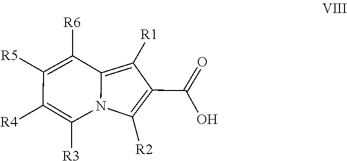
in which R1, R2, R3, R4, R5 and R6 are as defined for the compound of formula I, with a compound selected from 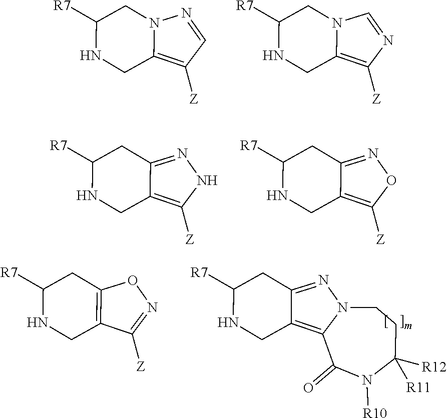
in which R7, R10, R11, R12, m and Z are as defined for the compound of formula I.

26. Method for the preparation of a compound of Formula I according to claim 25 by reacting a compound of Formula VIII 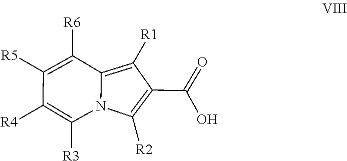
in which R1, R2, R3, R4, R5 and R6 are as defined for the compound of formula I, with a compound selected from 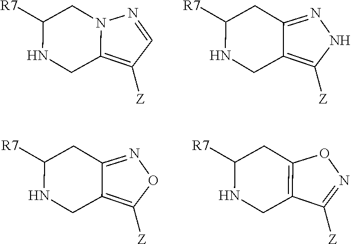
in which R7, R10, R11, R12, m and Z are as defined for the compound of formula I.


SEQUENCE LISTING (7 sequences)

SEQ ID NO: 1 - DNA, 24 bp, Artificial Sequence
  Antisense primer HBV
tgcagaggtgaagcgaagtgcaca

SEQ ID NO: 2 - DNA, 24 bp, Artificial Sequence
  Sense primer HBV
gacgtcctttgtttacgtcccgtc

SEQ ID NO: 3 - DNA, 25 bp, Artificial Sequence
  Hybprobe FL HBV
acggggcgcacctctctttacgcgg

SEQ ID NO: 4 - DNA, 26 bp, Artificial Sequence
  Hybprobe PH HBV
ctccccgtctgtgccttctcatctgc

SEQ ID NO: 5 - DNA, 21 bp, Artificial Sequence
  HBV forward primer
ctgtaccaaaccttcggacgg

SEQ ID NO: 6 - DNA, 19 bp, Artificial Sequence
  HBV reverse primer
aggagaaacgggctgaggc

SEQ ID NO: 7 - DNA, 26 bp, Artificial Sequence
  FAM-labelled probe for HBV
ccatcatcctgggctttcggaaaatt